SEQUENCE LISTING (75 sequences)

SEQ ID NO: 1 - DNA, 1321 bp, Homo sapiens
  CDS (87)..(1103)
ggcgctgggcagtgtggaggtcgttggagtcacttccgcgtcaccagctcctgtgcctgccagtcggtgcccctcccgctccagccatgctctccgccctcgcccggcctgccagcgctgctctccgccgcagcttcagcacctcggcccagaacaatgctaaagtagctgtgctaggggcctctggaggcatcgggcagccactttcacttctcctgaagaacagccccttggtgagccgcctgaccctctatgatatcgcgcacacacccggagtggccgcagatctgagccacatcgagaccaaagccgctgtgaaaggctacctcggacctgaacagctgcctgactgcctgaaaggttgtgatgtggtagttattccggctggagtccccagaaagccaggcatgacccgggacgacctgttcaacaccaatgccacgattgtggccaccctgaccgctgcctgtgcccagcactgcccggaagccatgatctgcgtcattgccaatccggttaattccaccatccccatcacagcagaagttttcaagaagcatggagtgtacaaccccaacaaaatcttcggcgtgacgaccctggacatcgtcagagccaacacctttgttgcagagctgaagggtttggatccagctcgagtcaacgtccctgtcattggtggccatgctgggaagaccatcatccccctgatctctcagtgcacccccaaggtggactttccccaggaccagctgacagcactcactgggcggatccaggaggccggcacggaggtggtcaaggctaaagccggagcaggctctgccaccctctccatggcgtatgccggcgcccgctttgtcttctcccttgtggatgcaatgaatggaaaggaaggtgttgtggaatgttccttcgttaagtcacaggaaacggaatgtacctacttctccacaccgctgctgcttgggaaaaagggcatcgagaagaacctgggcatcggcaaagtctcctcttttgaggagaagatgatctcggatgccatccccgagctgaaggcctccatcaagaagggggaagatttcgtgaagaccctgaagtgagccgctgtgacgggtggccagtttccttaatttatgaaggcatcatgtcactgcaaagccgttgcagataaactttgtattttaatttgctttggtgatgattactgtattgacatcatcatgccttccaaattgtgggtggctctgtgggcgcatcaataaaagccgtccttgattttatttttcaaggtcccttctgtaaaaaaaaaaaaaaaaaa

SEQ ID NO: 2 - protein, 338 aa, Homo sapiens
MLSALARPASAALRRSFSTSAQNNAKVAVLGASGGIGQPLSLLLKNSPLVSRLTLYDIAHTPGVAADLSHIETKAAVKGYLGPEQLPDCLKGCDVVVIPAGVPRKPGMTRDDLFNTNATIVATLTAACAQHCPEAMICVIANPVNSTIPITAEVFKKHGVYNPNKIFGVTTLDIVRANTFVAELKGLDPARVNVPVIGGHAGKTIIPLISQCTPKVDFPQDQLTALTGRIQEAGTEVVKAKAGAGSATLSMAYAGARFVFSLVDAMNGKEGVVECSFVKSQETECTYFSTPLLLGKKGIEKNLGIGKVSSFEEKMISDAIPELKASIKKGEDFVKTLK

SEQ ID NO: 3 - DNA, 2049 bp, Homo sapiens
  CDS (51)..(797)
gagcccagccgagcgtccgccgctgcccgtgcgcctctgcgctccgcgccatggccggcctcaactccctggaggcggtgaaacgcaagatccaggccctgcagcagcaggcggacgaggcggaagaccgcgcgcagggcctgcagcgggagctggacggcgagcgcgagcggcgcgagaaagctgaaggtgatgtggccgccctcaaccgacgcatccagctcgttgaggaggagttggacagggctcaggaacgactggccacggccctgcagaagctggaggaggcagaaaaagctgcagatgagagtgagagaggaatgaaggtgatagaaaaccgggccatgaaggatgaggagaagatggagattcaggagatgcagctcaaagaggccaagcacattgcggaagaggctgaccgcaaatacgaggaggtagctcgtaagctggtcatcctggagggtgagctggagagggcagaggagcgtgcggaggtgtctgaactaaaatgtggtgacctggaagaagaactcaagaatgttactaacaatctgaaatctctggaggctgcatctgaaaagtattctgaaaaggaggacaaatatgaagaagaaattaaacttctgtctgacaaactgaaagaggctgagacccgtgctgaatttgcagagagaacggttgcaaaactggaaaagacaattgatgacctggaagagaaacttgcccaggccaaagaagagaacgtgggcttacatcagacactggatcagacactaaacgaacttaactgtatataagcaaaacagaagagtcttgttccaacagaaactctggagctccgtgggtctttctcttctcttgtaagaagttccttttgttattgccatcttcgctttgctggaaatgtcaagcaaattatgaatacatgaccaaatattttgtatcggagaagctttgagcaccagttaaatctcattccttccctttttttttcaaatggcaccagctttttcagctctcttattttttccttaagtagcatttattcctaaggtaggcagggtatttcctagtaagcatactttcttaagacggaggccatttggttcctgggagaataggcagccccacactttgaagaatacagaccccagtatctagtcgtggatataattaaaacgctgaagaccataaccttttgggtcaactgttggtcaaactataggagagaccagggaccatcacatgggtagggattttccatccagagccaataaaaggactggtgggggccgggggtggctattgtgggaagtcataacccacagatagatcaacctaagaatcctggcccttctccactctccaccatgcaggacaaacatcttctcaagcagtcaacgtagaatgcttgggaaatagtcataattacccacatatagtaattaatagatggtaattaattgatccttgatgtgatgttcttttgcatatttccttcattctaaagttgttccctggccgggagcgtttgctttcgcctgtaatcccaacactttgggaggccaggacagatcacttgaggtcaggagttcgagaccagcccagccaacatggcgaaaccatgtctctactaaaaatacaaaaattatggtgacgcctgcctgtagtcccagctactcgggaggctgaggcaggaggatcgcttgaacccaggaagtggagactgcagtgagccgatatcgcaccacagcgctccagcctggtcgacagagtgagactccatctcaagaaaaaataaaaataaagttgttctctgaagagcaaatgtctcattccagtaatgacccactcagcaggaatatggtggagttcagtccaattcaggtcagccatatccaaaagaccacaagtcattactaagttgagcaaaagagtttttatctattagcagaaagggcctctctggcagcagagattaaaaactggcccaacttcatttccatacttcagggaacagcaaattgaggatttacttatctaggactt

SEQ ID NO: 4 - protein, 248 aa, Homo sapiens
MAGLNSLEAVKRKIQALQQQADEAEDRAQGLQRELDGERERREKAEGDVAALNRRIQLVEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKYSEKEDKYEEEIKLLSDKLKEAETRAEFAERTVAKLEKTIDDLEEKLAQAKEENVGLHQTLDQTLNELNCI

SEQ ID NO: 5 - DNA, 2251 bp, Homo sapiens
  CDS (154)..(1533)
gccgcgtgcagaggtgctcaagcctcctcgcggtccgcagtcagtgccgccgcgcccggcctcccgcacgccccgcaggtagcgcccccgcccgcggcccagagtgcgctcgcgccggcaccagctcccggataaacggcgcgccgcgcggagatgacagccgaggagatgaaggcgaccgagagcggggcgcagtcggcgccgctgcccatggagggagtggacatcagccccaaacaggacgaaggcgtgctgaaggtcatcaagagagagggcacaggtacagagatgcccatgattggggaccgagtctttgtccactacactggctggctattagatggcacaaagtttgactccagtctggatcgcaaggacaaattctcctttgacctgggaaaaggggaggtcatcaaggcttgggacattgccatagccaccatgaaggtgggggaggtgtgccacatcacctgcaaaccagaatatgcctacggttcagcaggcagtcctccaaagattccccccaatgccacgcttgtatttgaggtggagttgtttgagtttaagggagaagatctgacggaagaggaagatggcggaatcattcgcagaatacagactcgcggtgaaggctatgctaagcccaatgagggtgctatcgtggaggttgcactggaagggtactacaaggacaagctctttgaccagcgggagctccgctttgagattggcgagggggagaacctggatctgccttatggtctggagagggccattcagcgcatggagaaaggagaacattccatcgtgtacctcaagcccagctatgcttttggcagtgttgggaaggaaaagttccaaatcccaccaaatgctgagctgaaatatgaattacacctcaagagttttgaaaaggccaaggagtcttgggagatgaattcagaagagaagctggaacagagcaccatagtgaaagagcggggcactgtgtacttcaaggaaggtaaatacaagcaagctttactacagtataagaagatcgtgtcttggctggaatatgagtctagtttttccaatgaggaagcacagaaagcacaggcccttcgactggcctctcacctcaacctggccatgtgtcatctgaaactacaggccttctctgctgccattgaaagctgtaacaaggccctagaactggacagcaacaacgagaagggcctcttccgccggggagaggcccacctggccgtgaatgactttgaactggcacgggctgatttccagaaggtcctgcagctctaccccaacaacaaagccgccaagacccagctggctgtgtgccagcagcggatccgaaggcagcttgcccgggagaagaagctctatgccaatatgtttgagaggctggctgaggaggagaacaaggccaaggcagaggcttcctcaggagaccatcccactgacacagagatgaaggaggagcagaagagcaacacggcagggagccagtctcaggtggagacagaagcatagcccctctccaccagccctactcctgcggctgcctgccccccagtctccccactccaccctgttagttttgtaaaaactgaagaattttgagtgaattagacctttatttttctatctggttggatggtggctttaggggaagggggaaaggtgtaggctgggggattgaggtggggaatcattttagctggtgtcagcccctcttcccttcctccattgcacatgaacatatgtccatccatatatattcatcagaatgttaatttattttgctccctctgttaggtccattttctaagggtagaagaggcaagtggtagggatgaggtctgataagaacccagggtggagagggagactcctgggcagccgttttcctcatcctttccctctcccagtccatttccaaatgtggcctccatgtgggtgctagggacatgggaaaaaccactgctatgccatttcttctctctgttcccttcctcacccccgacggtgtggctgatgatgtcttctggtgtcatggtgaccaccccctgttccctgttctggtatttcccctgtcagtttcccctctcggccaggttgtgtcccaaaatcccctcagcctcttctctgcacgttgctgaaggtccaggcttgcctcaagttccatgcttgagcaataaagtggaaacaataaaacctgggtgtcagacaaaaaaaaaaaaaaaaaaaa

SEQ ID NO: 6 - protein, 459 aa, Homo sapiens
MTAEEMKATESGAQSAPLPMEGVDISPKQDEGVLKVIKREGTGTEMPMIGDRVFVHYTGWLLDGTKFDSSLDRKDKFSFDLGKGEVIKAWDIAIATMKVGEVCHITCKPEYAYGSAGSPPKIPPNATLVFEVELFEFKGEDLTEEEDGGIIRRIQTRGEGYAKPNEGAIVEVALEGYYKDKLFDQRELRFEIGEGENLDLPYGLERAIQRMEKGEHSIVYLKPSYAFGSVGKEKFQIPPNAELKYELHLKSFEKAKESWEMNSEEKLEQSTIVKERGTVYFKEGKYKQALLQYKKIVSWLEYESSFSNEEAQKAQALRLASHLNLAMCHLKLQAFSAAIESCNKALELDSNNEKGLFRRGEAHLAVNDFELARADFQKVLQLYPNNKAAKTQLAVCQQRIRRQLAREKKLYANMFERLAEEENKAKAEASSGDHPTDTEMKEEQKSNTAGSQSQVETEA

SEQ ID NO: 7 - DNA, 2562 bp, Homo sapiens
  CDS (56)..(1651)
gccgcgccggctctgggcactcagcatcgtttccttttcctccgctggagcagctatggcggcggtgaagaccctgaaccccaaggccgaggtggcccgagcgcaggcggcgctggcggtcaacatcagcgcagcgcggggtctgcaggacgtgctaaggaccaacctggggcccaagggcaccatgaagatgctcgtttctggcgctggagacatcaaacttactaaagacggcaatgtgctgcttcacgaaatgcaaattcaacacccaacagcttccttaatagcaaaggtagcaacagcccaggatgatataactggtgatggtacgacttctaatgtcctaatcattggagagctgctgaaacaggcggatctctacatttctgaaggccttcatcctagaataatcactgaaggatttgaagctgcaaaggaaaaggcccttcagtttttggaagaagtcaaagtaagcagagagatggacagggaaacacttatagatgtggccagaacatctcttcgtactaaagttcatgctgaacttgcagatgtcttaacagaggctgtagtggactccattttggccattaaaaagcaagatgaacctattgatctcttcatgattgagatcatggagatgaaacataaatctgaaactgatacaagcttaatcagagggcttgttttggaccacggagcacggcatcctgatatgaagaaaagggtggaggatgcatacatcctcacttgtaacgtgtcattagagtatgagaaaacagaagtgaattctggctttttttacaagagtgcagaagagagagaaaaactcgtgaaagctgaaagaaaattcattgaagatagggttaaaaaaataatagaactgaaaaggaaagtctgtggcgattcagataaaggatttgttgttattaatcaaaagggaattgaccccttttccttagatgctctttcaaaagaaggcatagtcgctctgcgcagagctaaaaggagaaatatggagaggctgactcttgcttgtggtggggtagccctgaattcttttgacgacctaagtcctgactgcttgggacatgcaggacttgtatatgagtatacattgggagaagagaagtttacctttattgagaaatgtaacaaccctcgttctgtcacattattgatcaaaggaccaaataagcacacactcactcagatcaaagatgcagtgagggacggcttgagggctgtcaaaaatgctattgatgatggctgtgtggttccaggtgctggtgccgtggaagtggcaatggcagaagccctgattaaacataagcccagtgtaaagggcagggcacagcttggagtccaagcatttgctgatgcattgctcattattcccaaggttcttgctcagaactctggttttgaccttcaggaaacattagttaaaattcaagcagaacattcagaatcaggtcagcttgtgggtgtggacctgaacacaggtgagccaatggtggcagcagaagtaggcgtatgggataactattgtgtaaagaaacagcttcttcactcctgcactgtgattgccaccaacattctcttggttgatgagatcatgcgagctggaatgtcttctctgaaaggttgaattgaagcttcctctgtatctgaatcttgaagactgcaaagtgatcctgaggattacagctgtggaatttttgtccaagcttcaaataattttgaaagaaattttcccatatgaaaaaaggagagaacactggcatctgttgaaatttggaagttctgaaattatagtatttttaaaaattgcactgaagtgtatacacataaagcaggtcttttatccagtgaacaggatgttttgctttagcagcagtgacataaaattccatgttagataagcatatgttacttaccttgttattaaatatttcttgaaaagcaaattttaatggttaattttatgtggacgtatgttaaattatccaaactaccctattgttaagcatttggttttaaaatttttatgctaatataaatgctcaagtaatttaaaatattgaaagcatccctgttggtataaatttctgagtaaatgcattggatcagttggactttgaacgccctttgaaatggctttgctaaaatgctcccgccacaaagttgtaggaaatgggaagaggagtcaactagaggcaagggagttgagagagctgcaactgtaaagggcaagaacaggcagaggtaaaaagatgatggaaggtgtggtgactaagggccacggttattgggtgaaatttgagatgtaggccaactgtattttcaagcttctgaacttaaggcaaaatattcatcgcaaagtctctagcgtcatatttttctcacccaaattacgtttccacgagttattatatatagttggtctatctctgcagtccttgaaggtgaagttgtgtgttactaggctgtgttttgggatgtcagcagtggcctgaagtgagttgtgcaataaatgttaagttgaaacctcaaaaaaaa

SEQ ID NO: 8 - protein, 531 aa, Homo sapiens
MAAVKTLNPKAEVARAQAALAVNISAARGLQDVLRTNLGPKGTMKMLVSGAGDIKLTKDGNVLLHEMQIQHPTASLIAKVATAQDDITGDGTTSNVLIIGELLKQADLYISEGLHPRIITEGFEAAKEKALQFLEEVKVSREMDRETLIDVARTSLRTKVHAELADVLTEAVVDSILAIKKQDEPIDLFMIEIMEMKHKSETDTSLIRGLVLDHGARHPDMKKRVEDAYILTCNVSLEYEKTEVNSGFFYKSAEEREKLVKAERKFIEDRVKKIIELKRKVCGDSDKGFVVINQKGIDPFSLDALSKEGIVALRRAKRRNMERLTLACGGVALNSFDDLSPDCLGHAGLVYEYTLGEEKFTFIEKCNNPRSVTLLIKGPNKHTLTQIKDAVRDGLRAVKNAIDDGCVVPGAGAVEVAMAEALIKHKPSVKGRAQLGVQAFADALLIIPKVLAQNSGFDLQETLVKIQAEHSESGQLVGVDLNTGEPMVAAEVGVWDNYCVKKQLLHSCTVIATNILLVDEIMRAGMSSLKG

SEQ ID NO: 9 - DNA, 2208 bp, Homo sapiens
  CDS (230)..(1486)
tctttggctttttttggcggagctggggcgccctccggaagcgtttccaactttccagaagtttctcgggacgggcaggagggggtggggactgccatatatagatcccgggagcaggggagcgggctaagagtagaatcgtgtcgcggctcgagagcgagagtcacgtcccggcgctagcccagcccgacccaggcccaccgtggtgcacgcaaaccacttcctggccatgcgctccctcctgcttctcagcgccttctgcctcctggaggcggccctggccgccgaggtgaagaaacctgcagccgcagcagctcctggcactgcggagaagttgagccccaaggcggccacgcttgccgagcgcagcgccggcctggccttcagcttgtaccaggccatggccaaggaccaggcagtggagaacatcctggtgtcacccgtggtggtggcctcgtcgctagggctcgtgtcgctgggcggcaaggcgaccacggcgtcgcaggccaaggcagtgctgagcgccgagcagctgcgcgacgaggaggtgcacgccggcctgggcgagctgctgcgctcactcagcaactccacggcgcgcaacgtgacctggaagctgggcagccgactgtacggacccagctcagtgagcttcgctgatgacttcgtgcgcagcagcaagcagcactacaactgcgagcactccaagatcaacttccgcgacaagcgcagcgcgctgcagtccatcaacgagtgggccgcgcagaccaccgacggcaagctgcccgaggtcaccaaggacgtggagcgcacggacggcgccctgctagtcaacgccatgttcttcaagccacactgggatgagaaattccaccacaagatggtggacaaccgtggcttcatggtgactcggtcctataccgtgggtgtcatgatgatgcaccggacaggcctctacaactactacgacgacgagaaggaaaagctgcaaatcgtggagatgcccctggcccacaagctctccagcctcatcatcctcatgccccatcacgtggagcctctcgagcgccttgaaaagctgctaaccaaagagcagctgaagatctggatggggaagatgcagaagaaggctgttgccatctccttgcccaagggtgtggtggaggtgacccatgacctgcagaaacacctggctgggctgggcctgactgaggccattgacaagaacaaggccgacttgtcacgcatgtcaggcaagaaggacctgtacctggccagcgtgttccacgccaccgcctttgagttggacacagatggcaacccctttgaccaggacatctacgggcgcgaggagctgcgcagccccaagctgttctacgccgaccaccccttcatcttcctagtgcgggacacccaaagcggctccctgctattcattgggcgcctggtccggcctaagggtgacaagatgcgagacgagttatagggcctcagggtgcacacaggatggcaggaggcatccaaaggctcctgagacacatgggtgctattggggttgggggggaggtgaggtaccagccttggatactccatggggtgggggtggaaaaacagaccggggttcccgtgtgcctgagcggaccttcccagctagaattcactccacttggacatgggccccagataccatgatgctgagcccggaaactccacatcctgtgggacctgggccatagtcattctgcctgccctgaaagtcccagatcaagcctgcctcaatcagtattcatatttatagccaggtaccttctcacctgtgagaccaaattgagctaggggggtcagccagccctcttctgacactaaaacacctcagctgcctccccagctctatcccaacctctcccaactataaaactaggtgctgcagcccctgggaccaggcacccccagaatgacctggccgcagtgaggcggattgagaaggagctcccaggaggggcttctgggcagactctggtcaagaagcatcgtgtctggcgttgtggggatgaactttttgttttgtttcttccttttttagttcttcaaagatagggagggaagggggaacatgagcctttgttgctatcaatccaagaacttatttgtacattttttttttcaataaaacttttccaatgacattttgttggagcgtggaaaaaa

SEQ ID NO: 10 - protein, 418 aa, Homo sapiens
MRSLLLLSAFCLLEAALAAEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 11 - DNA, 1690 bp, Homo sapiens
  CDS (77)..(1429)
ggcacgaggaaggtttttgctgcgccaacgcagtgaccgaaggctccgctcacgcccggcctgatcctgcctgaagatggtgccactggtggctgtggtatcagggccccgtgcccagctctttgcctgcctgctcaggctgggcactcagcaggtcggcccccttcagctgcacaccggggccagccatgcggccaggaaccattatgaggtgctggtgctgggtgggggcagtggcggaatcaccatggctgcccgcatgaagaggaaagtgggtgcagagaatgtggccattgttgagcccagtgagagacatttctaccagccaatctggacactggtgggtgctggtgccaaacaattgtcctcatctggtcgtcccacggcaagtgtgattccatctggtgtagaatggatcaaagctagagtgactgagttgaacccagacaagaactgcattcacacagatgacgacgagaagatctcctaccgatatcttattattgctctcggaatccagctggactatgagaagattaaaggcctacctgaaggtttcgctcatcccaaaatagggtcgaattattcagttaagactgtagagaagacatggaaagctctgcaggacttcaaagagggcaatgccatcttcaccttcccaaatactccagtgaagtgtgctggagcccctcagaagatcatgtacttatcagaagcctacttcaggaagacagggaagcgatccaaggccaatatcattttcaacacttctcttggagccattttcggggttaagaagtatgcagatgccctgcaggagatcatccaggagcggaacctcactgttaactacaagaaaaacctcattgaagtccgagccgataaacaagaggctgtatttgagaacctggacaaaccaggagagacccaagtgatttcatatgaaatgcttcatgtcacacctccaatgagcccaccagatgtcctcaagaccagtcctgtggctgatgctgctggttgggtggatgtggataaagaaactctgcaacacaggaggtacccaaatgtgtttgggattggggactgcaccaaccttcctacgtcaaagaccgctgctgcagtagctgcccagtcaggaatacttgataggacaatttctgtaattatgaagaatcaaacaccaacaaagaagtatgatggctacacatcatgtccactggtgaccggctacaaccgtgtgattcttgctgagtttgactacaaagcagagccgctagaaaccttcccctttgatcaaagcaaagagcgcctttccatgtatctcatgaaagctgacctgatgcctttcctgtattggaatatgatgctaaggggttactggggaggaccagcgtttctgcgcaagttgtttcatctaggtatgagttaaggatggctcagcacttgctcatcttggatggcttctgggccaaaactgcagtcactgaatgaccaagagcagcacgaaggacttggaacctatccttgtaaagagttccttgatgggtaatggtgaccaaatgcctcccttttcagtacctttgaacagcaaccatgtgggctactcatgatgggcttgattctttgggaataataaaatgaaataatacttttattttctgaataaaagtttgtcactgaaaaaaaaaaa

SEQ ID NO: 12 - protein, 450 aa, Homo sapiens
MVPLVAVVSGPRAQLFACLLRLGTQQVGPLQLHTGASHAARNHYEVLVLGGGSGGITMAARMKRKVGAENVAIVEPSERHFYQPIWTLVGAGAKQLSSSGRPTASVIPSGVEWIKARVTELNPDKNCIHTDDDEKISYRYLIIALGIQLDYEKIKGLPEGFAHPKIGSNYSVKTVEKTWKALQDFKEGNAIFTFPNTPVKCAGAPQKIMYLSEAYFRKTGKRSKANIIFNTSLGAIFGVKKYADALQEIIQERNLTVNYKKNLIEVRADKQEAVFENLDKPGETQVISYEMLHVTPPMSPPDVLKTSPVADAAGWVDVDKETLQHRRYPNVFGIGDCTNLPTSKTAAAVAAQSGILDRTISVIMKNQTPTKKYDGYTSCPLVTGYNRVILAEFDYKAEPLETFPFDQSKERLSMYLMKADLMPFLYWNMMLRGYWGGPAFLRKLFHLGMS

SEQ ID NO: 13 - DNA, 2593 bp, Homo sapiens
  CDS (49)..(2259)
ggccagcgcgtctgcttgttcgtgtgtgtgtcgttgcaggccttattcatgggctcaccgctgaggttcgacgggcgggtggtactggtcaccggcgcgggggcaggattgggccgagcctatgccctggcttttgcagaaagaggagcgttagttgttgtgaatgatttgggaggggacttcaaaggagttggtaaaggctccttagctgctgataaggttgttgaagaaataagaaggagaggtggaaaagcagtggccaactatgattcagtggaagaaggagagaaggttgtgaagacagccctggatgcttttggaagaatagatgttgtggtcaacaatgctggaattctgagggatcgttcctttgctaggataagtgatgaagactgggatataatccacagagttcatttgcggggttcattccaagtgacacgggcagcatgggaacacatgaagaaacagaagtatggaaggattattatgacttcatcagcttcaggaatatatggcaactttggccaggccaattatagtgctgcaaagttgggtcttctgggccttgcaaattctcttgcaattgaaggcaggaaaagcaacattcattgtaacaccattgctcctaatgcgggatcacggatgactcagacagttatgcctgaagatcttgtggaagccctgaagccagagtatgtggcacctcttgtcctttggctttgtcacgagagttgtgaggagaatggtggcttgtttgaggttggagcaggatggattggaaaattacgctgggagcggactcttggagctattgtaagacaaaagaatcacccaatgactcctgaggcagtcaaggctaactggaagaagatctgtgactttgagaatgccagcaagcctcagagtatccaagaatcaactggcagtataattgaagttctgagtaaaatagattcagaaggaggagtttcagcaaatcatactagtcgtgcaacgtctacagcaacatcaggatttgctggagctattggccagaaactccctccattttcttatgcttatacggaactggaagctattatgtatgcccttggagtgggagcgtcaatcaaggatccaaaagatttgaaatttatttatgaaggaagttctgatttctcctgtttgcccaccttcggagttatcataggtcagaaatctatgatgggtggaggattagcagaaattcctggactttcaatcaactttgcaaaggttcttcatggagagcagtacttagagttatataaaccacttcccagagcaggaaaattaaaatgtgaagcagttgttgctgatgtcctagataaaggatccggtgtagtgattattatggatgtctattcttattctgagaaggaacttatatgccacaatcagttctctctctttcttgttggctctggaggctttggtggaaaacggacatcagacaaagtcaaggtagctgtagccatacctaatagacctcctgatgctgtacttacagataccacctctcttaatcaggctgctttgtaccgcctcagtggagactggaatcccttacacattgatcctaactttgctagtctagcaggttttgacaagcccatattacatggattatgtacatttggattttctgccaggcgtgtgttacagcagtttgcagataatgatgtgtcaagattcaaggcaattaaggctcgttttgcaaaaccagtatatccaggacaaactctacaaactgagatgtggaaggaaggaaacagaattcattttcaaaccaaggtccaagaaactggagacattgtcatttcaaatgcatatgtggatcttgcaccaacatctggtacttcagctaagacaccctctgagggcgggaagcttcagagtacctttgtatttgaggaaataggacgccgcctaaaggatattgggcctgaggtggtgaagaaagtaaatgctgtatttgagtggcatataaccaaaggcggaaatattggggctaagtggactattgacctgaaaagtggttctggaaaagtgtaccaaggccctgcaaaaggtgctgctgatacaacaatcatactttcagatgaagatttcatggaggtggtcctgggcaagcttgaccctcagaaggcattctttagtggcaggctgaaggccagagggaacatcatgctgagccagaaacttcagatgattcttaaagactacgccaagctctgaagggcacactacactattaataaaaatggaatcattaaatactctcttcacccaaatatgcttgattattctgcaaaagtgattagaactaagatgcaggggaaattgcttaacattttcagatatcagataactgcagattttcattttctactaattttcatgtatcattatttttacaaggaactatatataagctagcacatgattatccttctgttcttagatctgtatcttcataataaaaaattttgcccaagtcctgtttccttagaatttgtgatagcattgataagttgaaaggaaaattaaatcaataaaggcctttgatacc

SEQ ID NO: 14 - protein, 736 aa, Homo sapiens
MGSPLRFDGRVVLVTGAGAGLGRAYALAFAERGALVVVNDLGGDFKGVGKGSLAADKVVEEIRRRGGKAVANYDSVEEGEKVVKTALDAFGRIDVVVNNAGILRDRSFARISDEDWDIIHRVHLRGSFQVTRAAWEHMKKQKYGRIIMTSSASGIYGNFGQANYSAAKLGLLGLANSLAIEGRKSNIHCNTIAPNAGSRMTQTVMPEDLVEALKPEYVAPLVLWLCHESCEENGGLFEVGAGWIGKLRWERTLGAIVRQKNHPMTPEAVKANWKKICDFENASKPQSIQESTGSIIEVLSKIDSEGGVSANHTSRATSTATSGFAGAIGQKLPPFSYAYTELEAIMYALGVGASIKDPKDLKFIYEGSSDFSCLPTFGVIIGQKSMMGGGLAEIPGLSINFAKVLHGEQYLELYKPLPRAGKLKCEAVVADVLDKGSGVVIIMDVYSYSEKELICHNQFSLFLVGSGGFGGKRTSDKVKVAVAIPNRPPDAVLTDTTSLNQAALYRLSGDWNPLHIDPNFASLAGFDKPILHGLCTFGFSARRVLQQFADNDVSRFKAIKARFAKPVYPGQTLQTEMWKEGNRIHFQTKVQETGDIVISNAYVDLAPTSGTSAKTPSEGGKLQSTFVFEEIGRRLKDIGPEVVKKVNAVFEWHITKGGNIGAKWTIDLKSGSGKVYQGPAKGAADTTIILSDEDFMEVVLGKLDPQKAFFSGRLKARGNIMLSQKLQMILKDYAKL

SEQ ID NO: 15 - DNA, 2113 bp, Homo sapiens
  CDS (63)..(1694)
gtgcggttgggaacgcggagcggacggattcgattcaacggggttccggaccgcgctgcgctatggagcaggtcaatgagctgaaggagaaaggcaacaaggccctgagcgtgggtaacatcgatgatgccttacagtgctactccgaagctattaagctggatccccacaaccacgtgctgtacagcaaccgttctgctgcctatgccaagaaaggagactaccagaaggcttatgaggatggctgcaagactgtcgacctaaagcctgactggggcaagggctattcacgaaaagcagcagctctagagttcttaaaccgctttgaagaagccaagcgaacctatgaggagggcttaaaacacgaggcaaataaccctcaactgaaagagggtttacagaatatggaggccaggttggcagagagaaaattcatgaaccctttcaacatgcctaatctgtatcagaagttggagagtgatcccaggacaaggacactactcagtgatcctacctaccgggagctgatagagcagctacgaaacaagccttctgacctgggcacgaaactacaagatccccggatcatgaccactctcagcgtcctccttggggtcgatctgggcagtatggatgaggaggaagagattgcaacacctccaccaccaccccctcccaaaaaggagaccaagccagagccaatggaagaagatcttccagagaataagaagcaggcactgaaagaaaaagagctggggaacgatgcctacaagaagaaagactttgacacagccttgaagcattacgacaaagccaaggagctggaccccactaacatgacttacattaccaatcaagcagcggtatactttgaaaagggcgactacaataagtgccgggagctttgtgagaaggccattgaagtggggagagaaaaccgagaagactatcgacagattgccaaagcatatgctcgaattggcaactcctacttcaaagaagaaaagtacaaggatgccatccatttctataacaagtctctggcagagcaccgaaccccagatgtgctcaagaaatgccagcaggcagagaaaatcctgaaggagcaagagcggctggcctacataaaccccgacctggctttggaggagaagaacaaaggcaacgagtgttttcagaaaggggactatccccaggccatgaagcattatacagaagccatcaaaaggaacccgaaagatgccaaattatacagcaatcgagctgcctgctacaccaaactcctggagttccagctggcactcaaggactgtgaggaatgtatccagctggagccgaccttcatcaagggttatacacggaaagccgctgcgctggaagcgatgaaggactacaccaaagccatggatgtgtaccagaaggcgctagacctggactccagctgtaaggaggcggcagacggctaccagcgctgtatgatggcgcagtacaaccggcacgacagccccgaagatgtgaagcgacgagccatggccgaccctgaggtgcagcagatcatgagtgacccagccatgcgccttatcctggaacagatgcagaaggacccccaggcactcagcgaacacttaaagaatcctgtaatagcacagaagatccagaagctgatggatgtgggtctgattgcaattcggtgatgacttgttcatccccccttcccttcgccctcatgtggaaagaggagctgggaccgcggcgagcagcacggagcggaagggagagcaggggagagaaggcctcatctctctatatttatacataaccccggggaagacacagagactcgtacctgcgctgtttgtgccgccgctgcctctgggccctcccagcacacgcatggtctcttcaccgctgccctcgagttccatgtctctttcccctgcccctagttgctgtctcggctgctctcccatagttggttttttttttatttggggcagtgggcatgttatggggaggggagggggttcttccagcctcaggtcccagctgtctcacgttgtttattctgcgtccccttctccaataaaacaagccagttgggcgtggttataac

SEQ ID NO: 16 - protein, 543 aa, Homo sapiens
MEQVNELKEKGNKALSVGNIDDALQCYSEAIKLDPHNHVLYSNRSAAYAKKGDYQKAYEDGCKTVDLKPDWGKGYSRKAAALEFLNRFEEAKRTYEEGLKHEANNPQLKEGLQNMEARLAERKFMNPFNMPNLYQKLESDPRTRTLLSDPTYRELIEQLRNKPSDLGTKLQDPRIMTTLSVLLGVDLGSMDEEEEIATPPPPPPPKKETKPEPMEEDLPENKKQALKEKELGNDAYKKKDFDTALKHYDKAKELDPTNMTYITNQAAVYFEKGDYNKCRELCEKAIEVGRENREDYRQIAKAYARIGNSYFKEEKYKDAIHFYNKSLAEHRTPDVLKKCQQAEKILKEQERLAYINPDLALEEKNKGNECFQKGDYPQAMKHYTEAIKRNPKDAKLYSNRAACYTKLLEFQLALKDCEECIQLEPTFIKGYTRKAAALEAMKDYTKAMDVYQKALDLDSSCKEAADGYQRCMMAQYNRHDSPEDVKRRAMADPEVQQIMSDPAMRLILEQMQKDPQALSEHLKNPVIAQKIQKLMDVGLIAIR

SEQ ID NO: 17 - DNA, 2033 bp, Homo sapiens
  CDS (29)..(1705)
ggacgagcagcggaggcggtcgggagcgatggtgaagatggcggcggcgggcggcggaggcggcggtggccgctactacggcggcggcagtgagggcggccgggcccctaagcggctcaagactgacaacgccggcgaccagcacggaggcggcggcggtggcggtggaggagccggggcggcgggcggcggcggcggtggggagaactacgatgacccgcacaaaacccctgcctccccagttgtccacatcaggggcctgattgacggtgtggtggaagcagaccttgtggaggccttgcaggagtttggacccatcagctatgtggtggtaatgcctaaaaagagacaagcactggtggagtttgaagatgtgttgggggcttgcaacgcagtgaactacgcagccgacaaccaaatatacattgctggtcacccagcttttgtcaactactctaccagccagaagatctcccgccctggggactcggatgactcccggagcgtgaacagtgtgcttctctttaccatcctgaaccccatttattcgatcaccacggatgttctttacactatctgtaatccttgtggccctgtccagagaattgtcattttcaggaagaatggagttcaggcgatggtggaatttgactcagttcaaagtgcccagcgggccaaggcctctctcaatggggctgatatctattctggctgttgcactctgaagatcgaatacgcaaagcctacacgcttgaatgtgttcaagaatgatcaggatacttgggactacacaaaccccaatctcagtggacaaggtgaccctggcagcaaccccaacaaacgccagaggcagccccctctcctgggagatcaccccgcagaatatggagggccccacggtgggtaccacagccattaccatgatgagggctacgggccccccccacctcactacgaagggagaaggatgggtccaccagtggggggtcaccgtcggggcccaagtcgctacggcccccagtatgggcaccccccaccccctcccccaccacccgagtatggccctcacgccgacagccctgtgctcatggtctatggcttggatcaatctaagatgaacggtgaccgagtcttcaatgtcttctgcttatatggcaatgtggagaaggtgaaattcatgaaaagcaagccgggggccgccatggtggagatggctgatggctacgctgtagaccgggccattacccacctcaacaacaacttcatgtttgggcagaagctgaatgtctgtgtctccaagcagccagccatcatgcctggtcagtcatacgggttggaagacgggtcttgcagttacaaagacttcagtgaatcccggaacaatcggttctccaccccagagcaggcagccaagaaccgcatccagcaccccagcaacgtgctgcacttcttcaacgccccgctggaggtgaccgaggagaacttctttgagatctgcgatgagctgggagtgaagcggccatcttctgtgaaagtattctcaggcaaaagtgagcgcagctcctctggactgctggagtgggaatccaagagcgatgccctggagactctgggcttcctgaaccattaccagatgaaaaacccaaatggtccatacccttacactctgaagttgtgtttctccactgctcagcacgcctcctaattaggtgcctaggaagagtcccatctgagcaggaagacatttctctttcctttatgccattttttgtttttgttatttgcaaaagatcttgtattcctttttttttttttttttttttaaatgctaggtttgtagaggcttacttaaccttaatggaaacgctggaaatctgcagggggagggagaggggaactgttatctcccaagattaaccttcacttttaaaaaattattgtacatgtgatttttttttttcctgttcatacatttgtgctgcccatgtactcttggcacatttcaataaaattgtttggaaaataaacacagc

SEQ ID NO: 18 - protein, 558 aa, Homo sapiens
MVKMAAAGGGGGGGRYYGGGSEGGRAPKRLKTDNAGDQHGGGGGGGGGAGAAGGGGGGENYDDPHKTPASPVVHIRGLIDGVVEADLVEALQEFGPISYVVVMPKKRQALVEFEDVLGACNAVNYAADNQIYIAGHPAFVNYSTSQKISRPGDSDDSRSVNSVLLFTILNPIYSITTDVLYTICNPCGPVQRIVIFRKNGVQAMVEFDSVQSAQRAKASLNGADIYSGCCTLKIEYAKPTRLNVFKNDQDTWDYTNPNLSGQGDPGSNPNKRQRQPPLLGDHPAEYGGPHGGYHSHYHDEGYGPPPPHYEGRRMGPPVGGHRRGPSRYGPQYGHPPPPPPPPEYGPHADSPVLMVYGLDQSKMNGDRVFNVFCLYGNVEKVKFMKSKPGAAMVEMADGYAVDRAITHLNNNFMFGQKLNVCVSKQPAIMPGQSYGLEDGSCSYKDFSESRNNRFSTPEQAAKNRIQHPSNVLHFFNAPLEVTEENFFEICDELGVKRPSSVKVFSGKSERSSSGLLEWESKSDALETLGFLNHYQMKNPNGPYPYTLKLCFSTAQHAS

SEQ ID NO: 19 - DNA, 3465 bp, Homo sapiens
  CDS (219)..(2639)
ctcgcgccaggcgagtctccgcgtctccctcgcgaactcggtgaaaggaattggcgccgttcgacaccaggcggatccgctctgcagcacgaacccatctccagccgcagccgcagccgccgcccgggccgaggagcagccgcagcagccgccaccagtggccgagtgagcggagccgagtttgaggcagcgcctagcggtgaatcggggccctcaccatgagttcctcgcctgttaatgtaaaaaagctgaaggtgtcggagctgaaagaggagctcaagaagcgacgcctttctgacaagggtctcaaggccgagctcatggagcgactccaggctgcgctggacgacgaggaggccgggggccgccccgccatggagcccgggaacggcagcctagacctgggcggggattccgctgggcgctcgggagcaggcctcgagcaggaggccgcggccggcggcgatgaagaggaggaggaagaggaagaggaggaggaaggaatctccgctctggacggcgaccagatggagctaggagaggagaacggggccgcgggggcggccgactcgggcccgatggaggaggaggaggccgcctcggaagacgagaacggcgacgatcagggtttccaggaaggggaagatgagctcggggacgaagaggaaggcgcgggcgacgagaacgggcacggggagcagcagcctcaaccgccggcgacgcagcagcaacagccccaacagcagcgcggggccgccaaggaggccgcggggaagagcagcggccccacctcgctgttcgcggtgacggtggcgccgcccggggcgaggcagggccagcagcaggcgggaggggacggcaaaacagaacagaaaggcggagataaaaagaggggtgttaaaagaccacgagaagatcatggccgtggatattttgagtacattgaagagaacaagtatagcagagccaaatctcctcagccacctgttgaagaagaagatgaacacttcgatgacacagtggtttgtcttgatacttataattgtgatctacattttaaaatatcaagagatcgtctcagtgcttcttcccttacaatggagagttttgcttttctttgggctggaggaagagcatcctatggtgtgtcaaaaggcaaagtgtgttttgagatgaaggttacagagaagatcccagtaaggcatttatatacaaaagatattgacatacatgaagttcgtattggctggtcactaactacaagtggaatgttacttggtgaagaagaattttcttatgggtattctctaaaaggaataaaaacatgcaactgtgagactgaagattatggagaaaagtttgatgaaaatgatgtgattacatgttttgctaactttgaaagtgatgaagtagaactctcgtatgctaagaatggacaagatcttggcgttgccttcaaaatcagtaaggaagttcttgctggacggccactgttcccgcatgttctctgccacaactgtgcagttgaatttaattttggtcagaaggaaaagccatattttccaatacctgaagagtatactttcatccagaacgtccccttagaggatcgagttagaggaccaaaggggcctgaagagaagaaagattgtgaagttgtgatgatgattggcttgccaggagctggaaaaactacctgggttactaaacatgcagcagaaaatccagggaaatataacattcttggcacaaatactattatggataagatgatggtggcaggttttaagaagcaaatggcagatactggaaaactgaacacactgttgcagagagccccccagtgtcttgggaaatttattgagattgctgcccgaaagaagcgaaattttattctggatcagacaaatgtgtctgctgctgcccagaggagaaaaatgtgcctgtttgcaggcttccagcgaaaagctgttgtagtttgcccaaaagatgaagactataagcaaagaacacagaagaaagcagaagtagaggggaaagacctaccagaacatgcggtcctcaaaatgaaaggaaactttaccctcccagaggtagctgagtgctttgatgaaataacctatgttgaacttcagaaggaagaagcccaaaaactcttggagcaatataaggaagaaagcaaaaaggctcttccaccagaaaagaaacagaacactggctcaaagaaaagcaataaaaataagagtggcaagaaccagtttaacagaggtggtggccatagaggacgtggaggattcaatatgcgtggtggaaatttcagaggaggagcccctgggaatcgtggcggatataataggaggggcaacatgccacagagaggtggtggcggtggaggaagtggtggaatcggctatccataccctcgtgcccctgtttttcctggccgtggtagttactcaaacagagggaactacaacagaggtggaatgcccaacagagggaactacaaccagaacttcagaggacgaggaaacaatcgtggctacaaaaatcaatctcagggctacaaccagtggcagcagggtcaattctggggtcagaagccatggagtcagcattatcaccaaggatattattgaatacccaaataaaacgaactgatacatatttctccaaaaccttcacaagaagtcgactgttttctttagtaggctaactttttaaacattccacaagaggaagtgcctgcgggttccttttttagaagctttgtgggttgattttttttcttttcttttttgtacatttttaattgcagtttaaaagtgaatcgtaagagaacctcagcattgtgcacgataagagaatgtgtcagtatttcagggttctacattttatctgtaaaatgtgacttttttttttttttatcacaacagaagtaaaatgttgctttgtacctggtgtcttttattaagaatttactccccccatttctcacagagaataacagtcgggagtcattgtcacaatataatagaaatgttagcaaccagattcatgtaaggactaagtggtcctcatgaattgcattaagactctgtactgctcatattacactccatcctctctgtagtttgctgggtagtggagggggtaagctaaatcatagtttctgacaataactgggaaggttttttcttaaaataacaatggaattggtataattgggattgaaaactaaaacttggaactaagatagagaagatggagtgtatgtagaagggctgttaaaaatgtaaaacttggttgcattatttgtggaggctcaaacttgtgaaggttaataccataatttttccatttgttctgcattttgattctgaaaagaaagctggctttgcccatttcttattaaaaaaacttgttgtaaaaaaaaaaaaaaaaaaaaaaaaaaaaaaaaaaaaa

SEQ ID NO: 20 - protein, 806 aa, Homo sapiens
MSSSPVNVKKLKVSELKEELKKRRLSDKGLKAELMERLQAALDDEEAGGRPAMEPGNGSLDLGGDSAGRSGAGLEQEAAAGGDEEEEEEEEEEEGISALDGDQMELGEENGAAGAADSGPMEEEEAASEDENGDDQGFQEGEDELGDEEEGAGDENGHGEQQPQPPATQQQQPQQQRGAAKEAAGKSSGPTSLFAVTVAPPGARQGQQQAGGDGKTEQKGGDKKRGVKRPREDHGRGYFEYIEENKYSRAKSPQPPVEEEDEHFDDTVVCLDTYNCDLHFKISRDRLSASSLTMESFAFLWAGGRASYGVSKGKVCFEMKVTEKIPVRHLYTKDIDIHEVRIGWSLTTSGMLLGEEEFSYGYSLKGIKTCNCETEDYGEKFDENDVITCFANFESDEVELSYAKNGQDLGVAFKISKEVLAGRPLFPHVLCHNCAVEFNFGQKEKPYFPIPEEYTFIQNVPLEDRVRGPKGPEEKKDCEVVMMIGLPGAGKTTWVTKHAAENPGKYNILGTNTIMDKMMVAGFKKQMADTGKLNTLLQRAPQCLGKFIEIAARKKRNFILDQTNVSAAAQRRKMCLFAGFQRKAVVVCPKDEDYKQRTQKKAEVEGKDLPEHAVLKMKGNFTLPEVAECFDEITYVELQKEEAQKLLEQYKEESKKALPPEKKQNTGSKKSNKNKSGKNQFNRGGGHRGRGGFNMRGGNFRGGAPGNRGGYNRRGNMPQRGGGGGGSGGIGYPYPRAPVFPGRGSYSNRGNYNRGGMPNRGNYNQNFRGRGNNRGYKNQSQGYNQWQQGQFWGQKPWSQHYHQGYY

SEQ ID NO: 21 - DNA, 3933 bp, Homo sapiens
  CDS (318)..(2861)
gcgagagtgggtttgttcttgggctgcagccgctgccgccgcttctcgccagcgccgttgctgcgggggattgtgggagtctccgcgtcccgctcgctgggagagaggtacctctccttttccctctccctttccctaagagttgtctgctggttctcagcttgaagaagattctgcagtccttattgatcctttttcttggcgttaccatttttgaagcaaagttaacctagctttctagtttgagctttctttttggccgtctttaaaaaaaattttttttttaatctataaaatagacaagagctagttctacaatgtccaagtcattccagcagtcatctctcagtagggactcacagggtcatgggcgtgacctgtctgcggcaggaataggccttcttgctgctgctacccagtctttaagtatgccagcatctcttggaaggatgaaccagggtactgcacgccttgctagtttaatgaatcttggaatgagttcttcattgaatcaacaaggagctcatagtgcactgtcttctgctagtacttcttcccataatttgcagtctatatttaacattggaagtagaggtccactccctttatcttctcaacaccgtggagatgcagaccaggccagtaacattttggccagctttggtctgtctgctagagacttagatgaactgagtcgttatccagaggacaagattactcctgagaatttgccccaaatccttctacagcttaaaaggaggagaactgaagaaggccctaccttgagttatggtagagatggcagatctgctacacgggagccaccatacagagtacctagggatgattgggaagaaaaaaggcactttagaagagatagttttgatgatcgtggtcctagtctcaacccagtgcttgattatgaccatggaagtcgttctcaagaatctggttattatgacagaatggattatgaagatgacagattaagagatggagaaaggtgtagggatgattctttttttggtgagacctcgcataactatcataaatttgacagtgagtatgagagaatgggacgtggtcctggccccttacaagagagatctctctttgagaaaaagagaggcgctcctccaagtagcaatattgaagacttccatggactcttaccgaagggttatccccatctgtgctctatatgtgatttgccagttcattctaataaggagtggagtcaacatatcaatggagcaagtcacagtcgtcgatgccagcttcttcttgaaatctacccagaatggaatcctgacaatgatacaggacacacaatgggtgatccattcatgttgcagcagtctacaaatccagcaccaggaattctgggacctccacctccctcatttcatcttgggggaccagcagttggaccaagaggaaatctgggtgctggaaatggaaacctgcaaggacctagacacatgcagaaaggcagagtggaaactagcagagttgttcacatcatggattttcaacgagggaaaaacttgagataccagctattacagctggtagaaccatttggagtcatttcaaatcatctgattctaaataaaattaatgaggcatttattgaaatggcaaccacagaggatgctcaggccgcagtggattattacacaaccacaccagcgttagtatttggcaagccagtgagagttcatttatcccagaagtataaaagaataaagaaacctgaaggaaagccagatcagaagtttgatcaaaagcaagagcttggacgtgtgatacatctcagcaatttgccgcattctggctattctgatagtgctgttctcaagcttgctgagccttatgggaaaataaagaattacatattgatgaggatgaaaagtcaggcttttattgagatggagacaagagaagatgcaatggcaatggttgaccattgtttgaaaaaagccctttggtttcaggggagatgtgtgaaggttgacctgtctgagaaatataaaaaactggttctgaggattccaaacagaggcattgatttactgaaaaaagataaatcccgaaaaagatcttactctccagatggcaaagaatctccaagtgataagaaatccaaaactgatggttcccagaagactgagagttcaaccgaaggtaaagaacaagaagagaagtccggtgaagatggtgagaaagacacaaaggatgaccagacagagcaggaacctaatatgcttcttgaatctgaagatgagctacttgtagatgaagaagaagcagcagcactgctagaaagtggcagttcagtgggagacgagaccgatcttgctaatttaggtgatgtggcttctgatgggaaaaaggaaccatcagataaagctgtgaaaaaagatggaagtgcttcagcagcagcaaagaaaaagcttaaaaaggtggacaagatcgaggaacttgatcaagaaaacgaagcagcgttggaaaatggaattaaaaatgaggaaaacacagaaccaggtgctgaatcttctgagaacgctgatgatcccaacaaagatacaagtgaaaacgcagatggtcaaagtgatgagaacaaggacgactatacaatcccagatgagtatagaattggaccatatcagcccaatgttcctgttggtatagactatgtgatacctaaaacagggttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaagatgtgcaaggagatttaatgatttcaaagaaaataatggttctttgtttttaatgttaaccttttttaaatacaatactgatagttagaagaaaactattgtactcttttgttttagtggagaaataatagatgtctgttcatgtgttaagtgttatagcaaaaaaaatacacatatggttaagttaatgaatagtttttgttttatcagaatggcaacagacagaagtactttgtagagattgacttcctaagctacttaagacaacttgcaccactaagaaaaaaatgtagaaccatttggaaaaatgaaatttagtagttccaagtttcaaagaaatgtcaacattttattccattcaataaagaacaaaaccaatagtgtttttattactttcatctgaaacattccatgttttaatctgagccttgcagactttcatttggagtttgaacccgttttggttgcatttcatttttggagaacttaattaacgtgagattggcaattgaaatgcaggtgcagttttctgttaatgtcatgctgttgtttaggtaataagaaatattaagtaattggctttagattttgtaatttttttccctgagttcctgctagatttcgtattctagtagtcaatgtattttcagtgaaatgcaaaaatattcccgttatctttgaccagtattaatttttgagatcttactgcttgtcacttgaatcccgtgattgtcatacatctctggtataagcaacatttgatttttgaagtgtgtagaccatctcttcatattttcaagatgtaattttacatttctgcatttttaaaacagtttggccataatcctagatgcacgcttctaattcatgtacctgcacatgtgacctttgtgaacagaaatttgcatgtataatttgtgtttacttgtaactttctggttatatactgcttatatctgtggattcaagttactgaagtgaataccaataaaaagaaaaccctaggccatgttaattggttatacatgtttggaatgttaaaaaaaaaaaaaaaaaaaaaaaaaaaaaaaa

SEQ ID NO: 22 - protein, 847 aa, Homo sapiens
MSKSFQQSSLSRDSQGHGRDLSAAGIGLLAAATQSLSMPASLGRMNQGTARLASLMNLGMSSSLNQQGAHSALSSASTSSHNLQSIFNIGSRGPLPLSSQHRGDADQASNILASFGLSARDLDELSRYPEDKITPENLPQILLQLKRRRTEEGPTLSYGRDGRSATREPPYRVPRDDWEEKRHFRRDSFDDRGPSLNPVLDYDHGSRSQESGYYDRMDYEDDRLRDGERCRDDSFFGETSHNYHKFDSEYERMGRGPGPLQERSLFEKKRGAPPSSNIEDFHGLLPKGYPHLCSICDLPVHSNKEWSQHINGASHSRRCQLLLEIYPEWNPDNDTGHTMGDPFMLQQSTNPAPGILGPPPPSFHLGGPAVGPRGNLGAGNGNLQGPRHMQKGRVETSRVVHIMDFQRGKNLRYQLLQLVEPFGVISNHLILNKINEAFIEMATTEDAQAAVDYYTTTPALVFGKPVRVHLSQKYKRIKKPEGKPDQKFDQKQELGRVIHLSNLPHSGYSDSAVLKLAEPYGKIKNYILMRMKSQAFIEMETREDAMAMVDHCLKKALWFQGRCVKVDLSEKYKKLVLRIPNRGIDLLKKDKSRKRSYSPDGKESPSDKKSKTDGSQKTESSTEGKEQEEKSGEDGEKDTKDDQTEQEPNMLLESEDELLVDEEEAAALLESGSSVGDETDLANLGDVASDGKKEPSDKAVKKDGSASAAAKKKLKKVDKIEELDQENEAALENGIKNEENTEPGAESSENADDPNKDTSENADGQSDENKDDYTIPDEYRIGPYQPNVPVGIDYVIPKTGFYCKLCSLFYTNEEVAKNTHCSSLPHYQKLKKFLNKLAEERRQKKET

SEQ ID NO: 23 - DNA, 1339 bp, Homo sapiens
  CDS (47)..(1018)
gaattccgattagtgtgatctcagctcaaggcaaaggtgggatatcatggcatctatctgggttggacaccgaggaacagtaagagattatccagactttagcccatcagtggatgctgaagctattcagaaagcaatcagaggaattggaactgatgagaaaatgctcatcagcattctgactgagaggtcaaatgcacagcggcagctgattgttaaggaatatcaagcagcatatggaaaggagctgaaagatgacttgaagggtgatctctctggccactttgagcatctcatggtggccctagtgactccaccagcagtctttgatgcaaagcagctaaagaaatccatgaagggcgcgggaacaaacgaagatgccttgattgaaatcttaactaccaggacaagcaggcaaatgaaggatatctctcaagcctattatacagtatacaagaagagtcttggagatgacattagttccgaaacatctggtgacttccggaaagctctgttgactttggcagatggcagaagagatgaaagtctgaaagtggatgagcatctggccaaacaagatgcccagattctctataaagctggtgagaacagatggggcacggatgaagacaaattcactgagatcctgtgtttaaggagctttcctcaattaaaactaacatttgatgaatacagaaatatcagccaaaaggacattgtggacagcataaaaggagaattatctgggcattttgaagacttactgttggccatagttaattgtgtgaggaacacgccggcctttttagccgaaagactgcatcgagccttgaagggtattggaactgatgagtttactctgaaccgaataatggtgtccagatcagaaattgaccttttggacattcgaacagagttcaagaagcattatggctattccctatattcagcaattaaatcggatacttctggagactatgaaatcacactcttaaaaatctgtggtggagatgactgaaccaagaagataatctccaaaggtccacgatgggctttcccaacagctccaccttacttcttctcatactatttaagagaacaagcaaatataaacagcaacttgtgttcctaacaggaattttcattgttctataacaacaacaacaaaagcgattattattttagagcatctcatttataatgtagcagctcataaatgaaattgaaaatggtattaaagatctgcaactactatccaacttatatttctgctttcaaagttaagaatctttatagttctactccattaaatataaagcaagataataaaacggaattc

SEQ ID NO: 24 - protein, 323 aa, Homo sapiens
MASIWVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGAGTNEDALIEILTTRTSRQMKDISQAYYTVYKKSLGDDISSETSGDFRKALLTLADGRRDESLKVDEHLAKQDAQILYKAGENRWGTDEDKFTEILCLRSFPQLKLTFDEYRNISQKDIVDSIKGELSGHFEDLLLAIVNCVRNTPAFLAERLHRALKGIGTDEFTLNRIMVSRSEIDLLDIRTEFKKHYGYSLYSAIKSDTSGDYEITLLKICGGDD

SEQ ID NO: 25 - DNA, 1659 bp, Homo sapiens
  CDS (152)..(1201)
gctgggggcgggtcctgcggcaccgcccgggaagctgcgcgagggtcgacagcctccgccacatcctccacctctcttggtccagcgagcgttgccgggccagggtcaagcggagggctccgacggcgcggacggagcgaagcgccgagccatggcgcaccaaacgggcatccacgccacggaagagctgaaggaattctttgccaaggcacgggctggctctgtgcggctcatcaaggttgtgattgaggacgagcagctcgtgctgggtgcctcgcaggagccagtaggccgctgggatcaggactatgacagggccgtgctgccactgctggacgcccagcagccctgctacctgctctaccgcctcgactcacagaatgctcagggcttcgaatggctcttcctcgcctggtcgcctgataactcccccgtgcggctgaagatgctgtacgcggccacgcgggccacagtgaaaaaggagtttggaggtggccacatcaaggatgagctcttcgggactgtgaaggatgacctctcttttgctgggtaccagaaacacctgtcgtcctgtgcggcacctgccccgctgacctcggctgagagagagctccagcagatccgcattaacgaggtgaagacagagatcagtgtggaaagcaagcaccagaccctgcagggcctcgccttccccctgcagcctgaggcccagcgggcactccagcagctcaagcagaaaatggtcaactacatccagatgaagctggacctagagcgggaaaccattgagctggtgcacacagagcccacggatgtggcccagctgccctcccgggtgccccgagatgctgcccgctaccacttcttcctctacaagcacacccatgagggcgacccccttgagtctgtagtgttcatctactccatgccggggtacaagtgcagcatcaaggagcgaatgctctactccagctgcaagagccgcctcctcgactccgtggagcaggacttccatctggagatcgccaagaaaattgagattggcgatggggcagagctgacggcagagttcctctacgacgaggtgcaccccaagcaacacgccttcaagcaggccttcgccaagcccaagggcccagggggcaagcggggccataagcgcctcatccgcggcccgggtgaaaatggggatgacagctaggaggctggagcagggccggccacgtgtggactgtggggctgcccaccttccgctccctgccaccatcctccttcctgggctccaggaaagtgtttctgggaggtcaggagggctggcagctgaacgcacttgcagcgtccgagggccaccgggctggcattttgtgacccttccctgttgctgtccctgcatctcgtctgtgtgcccagggtgtccggggaccctgcctggctggcttaagggggctgggtcaggggcctggcatgaacctggcctcccggggagctgagactagggtcccagcacagcccagaaacctttggccacaagaagtggggtcagtcagggctggggcaggggtcactgcagtttgggatggttgaatgctgtattttctaaagaataaaatatttttaaatcaagaaaaaaaaaaaaaaaaaaaaaaaaaaaaaaaaaaa

SEQ ID NO: 26 - protein, 349 aa, Homo sapiens
MAHQTGIHATEELKEFFAKARAGSVRLIKVVIEDEQLVLGASQEPVGRWDQDYDRAVLPLLDAQQPCYLLYRLDSQNAQGFEWLFLAWSPDNSPVRLKMLYAATRATVKKEFGGGHIKDELFGTVKDDLSFAGYQKHLSSCAAPAPLTSAERELQQIRINEVKTEISVESKHQTLQGLAFPLQPEAQRALQQLKQKMVNYIQMKLDLERETIELVHTEPTDVAQLPSRVPRDAARYHFFLYKHTHEGDPLESVVFIYSMPGYKCSIKERMLYSSCKSRLLDSVEQDFHLEIAKKIEIGDGAELTAEFLYDEVHPKQHAFKQAFAKPKGPGGKRGHKRLIRGPGENGDDS

SEQ ID NO: 27 - DNA, 2765 bp, Homo sapiens
  CDS (91)..(837)
gggagacgtggtgccgctgcgggctcgctctgccgtgcgctaggcttggtgggaaggcctgttctcgagtccgcgcttttcgtcaccgccatgtcgggaggtggtgtgattcgtggccccgcagggaacaacgattgccgcatctacgtgggtaacttacctccagacatccgaaccaaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataagatgattggtgacactttttgtagaacccatgttgtatacagttttcctttattcagtacaatcttttcattttttaattcaaactgttttgttcagaatgggctaaagtgttgaattgcattcttgtaatatccccttgctcctaacatctacattcccttcgtgtctttgataaattgtattttaagtgatgtcatagacaggattgtttaaatttagttaactccatactcttcagactgtgatattgtgtaaatgtctatctgccctggtttgtgtgaactgggatgttgggggtgtttgtggttatcttacctggggaagttcttatgtttatcttgcttttcatgtgtctttctgtagacatatctgaagagatggattaagaatgctttggattaaggattgtggagcacatttcaatcattttaggattgtcaaaaggaggattgaggaggatcagatcaataatggaggcaatggtatgactccaagtgctattgtcacagatgaaattggcagtattgaccttatactaaaaggcaggggttaaaaatgattatatacattttccttaaaacacttgcaaacattttattcagttgtctttagctacaattgctttgctttttaaaccttggcaattgtggcaaaattatattgcccattttgtagcaacttattttgctcccttccccccatttttgttttaatagggactaatgtgggaagaactggctaatttgtcacagtgcttagttacaactgttaatgtgtgacctgctgttggtgtacatgtgggtacagggtgtttttaaatccaacaagatagagtataatatcaatactgctaaatctgcatgtcctctgtgtgactgatagagcgttgctatttcatttttttaagacaaaatgaaagcaaaatatagagttccaatgtattggtgtagataatctagttgggaatacttttaagtctcaccttcccctttaaactaatattcataattggttcatatgtttaaaagactttaatttacaaattaaattgcaaatgggagcattagatttagttttagacttaggtgggtagcaatgccagtaaacttaaattacgtaacttcttgcaaccacgaaacctgtaatacgctgtacagtaacaagtgttggcattatcagttgaactgtaaatacaaaatgcttcttccaattagtctctatgatgattaagtttctaaaatttatctgaacaccattcagaaacttgttttggggaatttgatagttattgatgtgcatctgttaaactgatgacagacataactcatcattccccagaaaccttttttgattacagtatctaacattttgcctcctcttttttggttttgctggttataaaggtttggattggagagggctcactggatcccaatccttggagctggatcattggattcaaatcataatgtggataggatagggaggatgaattacccaggattcatggagcgggatcagattaccaggaacataggagtggattcctgccccaaccaaaccgcattcgtgtggatttttttattcaacttaattggctattccaaagattttttttttcctatttttgacgattggagcccttaagatgcacgatggaattgtgttttgcgttttttggtaaaaggagcaaagcgaggacctggagataaacgctggagcaatctccttggaaggattcagcacgagtagatggtaaacatttaaaggggaaagggggggtttgtttaaaatagtaaatcagtaagtcacttctaaatttaaagaaaacaaaattggagttgaagaataagtaggtttccaattggctattgccgttttctttgaaaaaataaacattttttaaaaaactaaaaaaaaaaaaaaa

SEQ ID NO: 28 - protein, 248 aa, Homo sapiens
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

SEQ ID NO: 29 - DNA, 1167 bp, Homo sapiens
  CDS (73)..(966)
gccaacttctccagccggccggggcgagcgccagcgcggcgtccggggcgagtgacacgcagagctgaagccatggttcatcaggtgctctaccgggcgctggtctccaccaagtggctggcggagtccatcaggactggcaagctggggcccggcctgcgggtgctggacgcgtcctggtactcaccaggcacccgagaggcccgcaaggagtacctcgagcgccacgtacccggcgcctctttctttgacatagaagagtgccgggacacggcgtcgccctacgagatgatgctgcccagcgaggctggcttcgccgagtatgtgggccgcctgggcatcagcaaccacacgcacgtggtggtgtatgatggtgaacacctgggcagcttctatgctccccgggtctggtggatgttccgtgtgtttggccaccgcaccgtatcagtgctcaatggtggcttccggaactggctgaaggagggccacccggtgacatccgagccctcacgcccagaaccggccgtcttcaaagccacactggaccgctccctgctcaagacctacgagcaggtgctggagaaccttgaatctaagaggttccagctggtggattcaaggtctcaagggcggttcctgggcaccgagccggagccggatgcagtaggactggactcgggccatatccgtggtgccgtcaacatgcctttcatggacttcctgactgaggatggcttcgagaagggcccagaagagctccgtgctctgttccagaccaagaaggtggatctctcgcagcctctcattgccacgtgccgcaagggagtcaccgcctgccacgtggccttggctgcctacctctgcggcaagcctgatgtggccgtgtacgatggctcctggtccgagtggtttcgccgggcccccccagagagccgtgtgtcccagggaaagtctgagaaggcctgagccgtgacctcttctgcttactgtaactgcggccggtttagtgaccccatgacttacagccggttcttacctcttaggtgaaggagatgacatgtttttagaattgctgtgcaaggctcaccctctctctgtcaacactggaataaactttgccttttctgaaaaaaaaaaaaaaaaaaaaaaaaaaaaaaaaaaaaaaaa

SEQ ID NO: 30 - protein, 297 aa, Homo sapiens
MVHQVLYRALVSTKWLAESIRTGKLGPGLRVLDASWYSPGTREARKEYLERHVPGASFFDIEECRDTASPYEMMLPSEAGFAEYVGRLGISNHTHVVVYDGEHLGSFYAPRVWWMFRVFGHRTVSVLNGGFRNWLKEGHPVTSEPSRPEPAVFKATLDRSLLKTYEQVLENLESKRFQLVDSRSQGRFLGTEPEPDAVGLDSGHIRGAVNMPFMDFLTEDGFEKGPEELRALFQTKKVDLSQPLIATCRKGVTACHVALAAYLCGKPDVAVYDGSWSEWFRRAPPESRVSQGKSEKA

SEQ ID NO: 31 - DNA, 2110 bp, Homo sapiens
  CDS (48)..(1346)
ctgaggcccagcccccttcgcccgtttccatcacgagtgccgccagcatgtctgacaaactgccctacaaagtcgccgacatcggcctggctgcctggggacgcaaggccctggacattgctgagaacgagatgccgggcctgatgcgtatgcgggagcggtactcggcctccaagccactgaagggcgcccgcatcgctggctgcctgcacatgaccgtggagacggccgtcctcattgagaccctcgtcaccctgggtgctgaggtgcagtggtccagctgcaacatcttctccacccaggaccatgcggcggctgccattgccaaggctggcattccggtgtatgcctggaagggcgaaacggacgaggagtacctgtggtgcattgagcagaccctgtacttcaaggacgggcccctcaacatgattctggacgacgggggcgacctcaccaacctcatccacaccaagtacccgcagcttctgccaggcatccgaggcatctctgaggagaccacgactggggtccacaacctctacaagatgatggccaatgggatcctcaaggtgcctgccatcaatgtcaatgactccgtcaccaagagcaagtttgacaacctctatggctgccgggagtccctcatagatggcatcaagcgggccacagatgtgatgattgccggcaaggtagcggtggtagcaggctatggtgatgtgggcaagggctgtgcccaggccctgcggggtttcggagcccgcgtcatcatcaccgagattgaccccatcaacgcactgcaggctgccatggagggctatgaggtgaccaccatggatgaggcctgtcaggagggcaacatctttgtcaccaccacaggctgtattgacatcatccttggccggcactttgagcagatgaaggatgatgccattgtgtgtaacattggacactttgacgtggagatcgatgtcaagtggctcaacgagaacgccgtggagaaggtgaacatcaagccgcaggtggaccggtatcggttgaagaatgggcgccgcatcatcctgctggccgagggtcggctggtcaacctgggttgtgccatgggccaccccagcttcgtgatgagtaactccttcaccaaccaggtgatggcgcagatcgagctgtggacccatccagacaagtaccccgttggggttcatttcctgcccaagaagctggatgaggcagtggctgaagcccacctgggcaagctgaatgtgaagttgaccaagctaactgagaagcaagcccagtacctgggcatgtcctgtgatggccccttcaagccggatcactaccgctactgagagccaggtctgcgtttcaccctccagctgctgtccttgcccaggccccacctctcctccctaagagctaatggcaccaactttgtgactggtttgtcagtgtcccccatcgactctctggggctgatcacttagtttttggcctctgctgcagccgtcatactgttccaaatgtggcagcgggaacagagtaccctcttcaagccccggtcatgatggaggtcccagccacagggaaccatgagctcagtggtcttggaacagctcactaagtcagtccttccttagcctggaagccagtagtggagtcacaaagcccatgtgttttgccatctaggccttcacctggtctgtggacttatacctgtgtgcttggtttacaggtccagtggttcttcagcccatgacagatgagaaggggctatattgaagggcaaagaggaactgttgtttgaattttcctgagagcctggcttagtgctgggccttctcttaaacctcattacaatgaggttagtacttttagtccctgttttacaggggttagaatagactgttaaggggcaactgagaaagaacagagaagtgacagctaggggttgagaggggccagaaaaacatgaatgcaggcagatttcgtgaaatctgccaccactttataaccagatggttcctttcacaaccctgggtcaaaaagagaataatttggcctataatgttaaaagaaagcaggaaggtgggtaaataaaaatcttggtgcctgg

SEQ ID NO: 32 - protein, 432 aa, Homo sapiens
MSDKLPYKVADIGLAAWGRKALDIAENEMPGLMRMRERYSASKPLKGARIAGCLHMTVETAVLIETLVTLGAEVQWSSCNIFSTQDHAAAAIAKAGIPVYAWKGETDEEYLWCIEQTLYFKDGPLNMILDDGGDLTNLIHTKYPQLLPGIRGISEETTTGVHNLYKMMANGILKVPAINVNDSVTKSKFDNLYGCRESLIDGIKRATDVMIAGKVAVVAGYGDVGKGCAQALRGFGARVIITEIDPINALQAAMEGYEVTTMDEACQEGNIFVTTTGCIDIILGRHFEQMKDDAIVCNIGHFDVEIDVKWLNENAVEKVNIKPQVDRYRLKNGRRIILLAEGRLVNLGCAMGHPSFVMSNSFTNQVMAQIELWTHPDKYPVGVHFLPKKLDEAVAEAHLGKLNVKLTKLTEKQAQYLGMSCDGPFKPDHYRY

SEQ ID NO: 33 - DNA, 1698 bp, Homo sapiens
  CDS (191)..(1309)
cccggccctccctgcacggcctcccgtgcgcccctgtcagactgtggcggccggtcgcgcggtgcgctctccctccctgcccgcagcctggagaggcgcttcgtgctgcacacccccgcgttcctgccggcaccgcgcctgccctctgccgcgctccgccctgccgccgaccgcacgcccgccgcgggacatggcacacgcaccggcacgctgccccagcgcccggggctccggggacggcgagatgggcaagcccaggaacgtggcgctcatcaccggtatcacaggccaggatggttcctacctggctgagttcctgctggagaaaggctatgaggtccatggaattgtacggcggtccagttcatttaatacgggtcgaattgagcatctgtataagaatccccaggctcacattgaaggaaacatgaagttgcactatggcgatctcactgacagtacctgccttgtgaagatcattaatgaagtaaagcccacagagatctacaaccttggagcccagagccacgtcaaaatttcctttgacctcgctgagtacactgcggacgttgacggagttggcactctacgacttctagatgcagttaagacttgtggccttatcaactctgtgaagttctaccaagcctcaacaagtgaactttatgggaaagtgcaggaaataccccagaaggagaccacccctttctatccccggtcaccctatggggcagcaaaactctatgcctattggattgtggtgaacttccgtgaggcgtataatctctttgcagtgaacggcattctcttcaatcatgagagtcccagaagaggagctaatttcgttactcgaaaaattagccggtcagtagctaagatttaccttggacaactggaatgtttcagtttgggaaatctggatgccaaacgagattggggccatgccaaggactatgtggaggctatgtggttgatgttgcagaatgatgagccggaggacttcgttatagctactggggaggtccatagtgtccgggaatttgtcgagaaatcattcttgcacattggaaaaaccattgtgtgggaaggaaagaatgaaaatgaagtgggcagatgtaaagagaccggcaaagttcacgtgactgtggatctcaagtactaccggccaactgaagtggactttctgcagggcgactgcaccaaagcgaaacagaagctgaactggaagccccgggtcgctttcgatgagctggtgagggagatggtgcacgccgacgtggagctcatgaggacaaaccccaatgcctgagcagcgcctcggagcccggcccgccctccggctacaatccccgcagagtctccggtgcagacgcgctgcggggatggggagcggcgtgccaatctgcgggtcccctgcggcccctgctgccgctgcgctgtcccggccgcaagagcggggccgccccgccgaggtttgtagcagccgggatgtgaccctccagggtttgggtcgctttgcgtttgtcgaagcctcctctgaatggctttgtgaaatcaagatgttttaatcacattcactttacttgaaattatgttgttacacaacaaattgtggggccttcaaattgtttttctcttttcatattaaaaatggtctttctgtgaactagcaaaaaaaaaaaaaaaaaaaaaaaaaaa

SEQ ID NO: 34 - protein, 372 aa, Homo sapiens
MAHAPARCPSARGSGDGEMGKPRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 35 - DNA, 2963 bp, Homo sapiens
  CDS (26)..(2317)
ccactgctgtcctcttcagctcaagatggtggcctgccgggcgattggcatcctcagccgcttttctgccttcaggatcctccgctcccgaggttatatatgccgcaattttacagggtcttctgctttgctgaccagaacccatattaactatggagtcaaaggggatgtggcagttgttcgaattaactctcccaattcaaaggtaaatacactgagtaaagagctacattcagagttctcagaagttatgaatgaaatctgggctagtgatcaaatcagaagtgccgtccttatctcatcaaagccaggctgctttattgcaggtgctgatatcaacatgttagccgcttgcaagacccttcaagaagtaacacagctatcacaagaagcacagagaatagttgagaaacttgaaaagtccacaaagcctattgtggctgccatcaatggatcctgcctgggaggaggacttgaggttgccatttcatgccaatacagaatagcaacaaaagacagaaaaacagtattaggtacccctgaagttttgctgggggccttaccaggagcaggaggcacacaaaggctgcccaaaatggtgggtgtgcctgctgctttggacatgatgctgactggtagaagcattcgtgcagacagggcaaagaaaatgggactggttgaccaactggtggaacccctgggaccaggactaaaacctccagaggaacggacaatagaatacctagaagaagttgcaattacttttgccaaaggactagctgataagaagatctctccaaagagagacaagggattggtggaaaaattgacagcgtatgccatgactattccatttgtcaggcaacaggtttacaaaaaagtggaagaaaaagtgcgaaagcagactaaaggcctttatcctgcacctctgaaaataattgatgtggtaaagactggaattgagcaagggagtgatgccggttatctctgtgaatctcagaaatttggagagcttgtaatgaccaaagaatcaaaggccttgatgggactctaccatggtcaggtcctgtgcaagaagaataaatttggagctccacagaaggatgttaagcatctggctattcttggtgcagggctgatgggagcaggcatcgcccaagtctccgtggataaggggctaaagactatacttaaagatgccaccctcactgcgctagaccgaggacagcaacaagtgttcaaaggattgaatgacaaagtgaagaagaaagctctaacatcatttgaaagggattccatcttcagcaacttgactgggcagcttgattaccaaggttttgaaaaggccgacatggtgattgaagctgtgtttgaggaccttagtcttaagcacagagtgctaaaggaagtagaagcggtgattccagatcactgtatctttgccagtaacacatctgctctcccaatcagtgaaatcgctgctgtcagcaaaagacctgagaaggtgattggcatgcactacttctctcccgtggacaagatgcagctgctggagattatcacgaccgagaaaacttccaaagacaccagtgcttcagctgtagcagttggtctcaagcaggggaaggtcatcattgtggttaaggatggacctggcttctatactaccaggtgtcttgcgcccatgatgtctgaagtcatccgaatcctccaggaaggagttgacccgaagaagctggattccctgaccacaagctttggctttcctgtgggtgccgccacactggtggatgaagttggtgtggatgtagcgaaacatgtggcggaagatctgggcaaagtctttggggagcggtttggaggtggaaacccagaactgctgacacagatggtgtccaagggcttcctaggtcgtaaatctgggaagggcttttacatctatcaggagggtgtgaagaggaaggatttgaattctgacatggatagtattttagcgagtctgaagctgcctcctaagtctgaagtctcatcagacgaagacatccagttccgcctggtgacaagatttgtgaatgaggcagtcatgtgcctgcaagaggggatcttggccacacctgcagagggagacatcggagccgtctttgggcttggcttcccgccttgtctgggagggcctttccgctttgtggatctgtatggcgcccagaagatagtggaccggctcaagaaatatgaagctgcctatggaaaacagttcaccccatgccagctgctagctgaccatgctaacagccctaacaagaagttctaccagtgagcaggcctcatgcctcgctcagtcagtgcactaaccccagctgccggcagtgctggttctccaacagagtggtgtctagatttatcagagtaacgagaagacaaactccggcactgggtttgctccctgattaaagtgccttcagccaagaccatctctccctcctggtgaagtgtgacttcgaattagtttgcacttcctattggaaggtagagcccactgctcattgtataagccccgaggcctagagtggcagccaagagccatctgaagccacctctctgcctgttcctcccaagaggccagggtggccaggggtggtgagggcagttctgcacccagccaaacacataacaataaaaaccaaactctgtgtcagcatctttgcccttctggtttaaacgcctccttcaaaaagcaatctggaagaaagccctgtgctttgggggagtaagaatgtgtgtgcagaattctaggcagcaccttagggagggactgggatgagagaaagtgggacctggtgggctcaaccacacacacctgtctgtgcagatgctttgcccaggcttctcaccacggtgtaccgggatattaaacctctttccccagcaaaaaaaaaaaaaaaaaaaaaaaaaaaaaaaaaaaa

SEQ ID NO: 36 - protein, 763 aa, Homo sapiens
MVACRAIGILSRFSAFRILRSRGYICRNFTGSSALLTRTHINYGVKGDVAVVRINSPNSKVNTLSKELHSEFSEVMNEIWASDQIRSAVLISSKPGCFIAGADINMLAACKTLQEVTQLSQEAQRIVEKLEKSTKPIVAAINGSCLGGGLEVAISCQYRIATKDRKTVLGTPEVLLGALPGAGGTQRLPKMVGVPAALDMMLTGRSIRADRAKKMGLVDQLVEPLGPGLKPPEERTIEYLEEVAITFAKGLADKKISPKRDKGLVEKLTAYAMTIPFVRQQVYKKVEEKVRKQTKGLYPAPLKIIDVVKTGIEQGSDAGYLCESQKFGELVMTKESKALMGLYHGQVLCKKNKFGAPQKDVKHLAILGAGLMGAGIAQVSVDKGLKTILKDATLTALDRGQQQVFKGLNDKVKKKALTSFERDSIFSNLTGQLDYQGFEKADMVIEAVFEDLSLKHRVLKEVEAVIPDHCIFASNTSALPISEIAAVSKRPEKVIGMHYFSPVDKMQLLEIITTEKTSKDTSASAVAVGLKQGKVIIVVKDGPGFYTTRCLAPMMSEVIRILQEGVDPKKLDSLTTSFGFPVGAATLVDEVGVDVAKHVAEDLGKVFGERFGGGNPELLTQMVSKGFLGRKSGKGFYIYQEGVKRKDLNSDMDSILASLKLPPKSEVSSDEDIQFRLVTRFVNEAVMCLQEGILATPAEGDIGAVFGLGFPPCLGGPFRFVDLYGAQKIVDRLKKYEAAYGKQFTPCQLLADHANSPNKKFYQ

SEQ ID NO: 37 - DNA, 2438 bp, Homo sapiens
  CDS (45)..(1571)
cccggcggcgccaaccgaagcgccccgcctgatccgtgtccgacatgctgcgccgcgctctgctgtgcctggccgtggccgccctggtgcgcgccgacgcccccgaggaggaggaccacgtcctggtgctgcggaaaagcaacttcgcggaggcgctggcggcccacaagtacctgctggtggagttctatgccccttggtgtggccactgcaaggctctggcccctgagtatgccaaagccgctgggaagctgaaggcagaaggttccgagatcaggttggccaaggtggacgccacggaggagtctgacctggcccagcagtacggcgtgcgcggctatcccaccatcaagttcttcaggaatggagacacggcttcccccaaggaatatacagctggcagagaggctgatgacatcgtgaactggctgaagaagcgcacgggcccggctgccaccaccctgcctgacggcgcagctgcagagtccttggtggagtccagcgaggtggctgtcatcggcttcttcaaggacgtggagtcggactctgccaagcagtttttgcaggcagcagaggccatcgatgacataccatttgggatcacttccaacagtgacgtgttctccaaataccagctcgacaaagatggggttgtcctctttaagaagtttgatgaaggccggaacaactttgaaggggaggtcaccaaggagaacctgctggactttatcaaacacaaccagctgccccttgtcatcgagttcaccgagcagacagccccgaagatttttggaggtgaaatcaagactcacatcctgctgttcttgcccaagagtgtgtctgactatgacggcaaactgagcaacttcaaaacagcagccgagagcttcaagggcaagatcctgttcatcttcatcgacagcgaccacaccgacaaccagcgcatcctcgagttctttggcctgaagaaggaagagtgcccggccgtgcgcctcatcaccctggaggaggagatgaccaagtacaagcccgaatcggaggagctgacggcagagaggatcacagagttctgccaccgcttcctggagggcaaaatcaagccccacctgatgagccaggagctgccggaggactgggacaagcagcctgtcaaggtgcttgttgggaagaactttgaagacgtggcttttgatgagaaaaaaaacgtctttgtggagttctatgccccatggtgtggtcactgcaaacagttggctcccatttgggataaactgggagagacgtacaaggaccatgagaacatcgtcatcgccaagatggactcgactgccaacgaggtggaggccgtcaaagtgcacagcttccccacactcaagttctttcctgccagtgccgacaggacggtcattgattacaacggggaacgcacgctggatggttttaagaaattcctggagagcggtggccaggatggggcaggggatgatgacgatctcgaggacctggaagaagcagaggagccagacatggaggaagacgatgatcagaaagctgtgaaagatgaactgtaatacgcaaagccagacccgggcgctgccgagacccctcgggggctgcacacccagcagcagcgcacgcctccgaagcctgcggcctcgcttgaaggagggcgtcgccggaaacccagggaacctctctgaagtgacacctcacccctacacaccgtccgttcacccccgtctcttccttctgcttttcggtttttggaaagggatccatctccaggcagcccaccctggtggggcttgtttcctgaaaccatgatgtactttttcatacatgagtctgtccagagtgcttgctaccgtgttcggagtctcgctgcctccctcccgcgggaggtttctcctctttttgaaaattccgtctgtgggatttttagacatttttcgacatcagggtatttgttccaccttggccaggcctcctcggagaagcttgtcccccgtgtgggagggacggagccggactggacatggtcactcagtaccgcctgcagtgtcgccatgactgatcatggctcttgcatttttgggtaaatggagacttccggatcctgtcagggtgtcccccatgcctggaagaggagctggtggctgccagccctggggcccggcacaggcctgggccttccccttccctcaagccagggctcctcctcctgtcgtgggctcattgtgaccactggcctctctacagcacggcctgtggcctgttcaaggcagaaccacgacccttgactcccgggtggggaggtggccaaggatgctggagctgaatcagacgctgacagttcttcaggcatttctatttcacaatcgaattgaacacattggccaaataaagttgaaattttaccaccaaaaaaaaaaaaaaaa

SEQ ID NO: 38 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 39 - DNA, 959 bp, Homo sapiens
  CDS (120)..(764)
gcagtggaggcggcccaggcccgccttccgcagggtgtcgccgctgtgccgctagcggtgccccgcctgctgcggtggcaccagccaggaggcggagtggaagtggccgtggggcgggtatgggactagctggcgtgtgcgccctgagacgctcagcgggctatatactcgtcggtggggccggcggtcagtctgcggcagcggcagcaagacggtgcagtgaaggagagtgggcgtctggcggggtccgcagtttcagcagagccgctgcagccatggccccaatcaaggtgggagatgccatcccagcagtggaggtgtttgaaggggagccagggaacaaggtgaacctggcagagctgttcaagggcaagaagggtgtgctgtttggagttcctggggccttcacccctggatgttccaagacacacctgccagggtttgtggagcaggctgaggctctgaaggccaagggagtccaggtggtggcctgtctgagtgttaatgatgcctttgtgactggcgagtggggccgagcccacaaggcggaaggcaaggttcggctcctggctgatcccactggggcctttgggaaggagacagacttattactagatgattcgctggtgtccatctttgggaatcgacgtctcaagaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccctgtgctggggccctgcaattggaatgttggccagatttctgcaataaacacttgtggtttgcggccaaaaaaaaaaaaaaaaaaaaaaaaaaaaaaaaaaaaaaaaaaaaaaaaaaaaaaaaaaaaaaaaaaaaaaaaaaaa

SEQ ID NO: 40 - protein, 214 aa, Homo sapiens
MGLAGVCALRRSAGYILVGGAGGQSAAAAARRCSEGEWASGGVRSFSRAAAAMAPIKVGDAIPAVEVFEGEPGNKVNLAELFKGKKGVLFGVPGAFTPGCSKTHLPGFVEQAEALKAKGVQVVACLSVNDAFVTGEWGRAHKAEGKVRLLADPTGAFGKETDLLLDDSLVSIFGNRRLKRFSMVVQDGIVKALNVEPDGTGLTCSLAPNIISQL

SEQ ID NO: 41 - DNA, 1874 bp, Homo sapiens
  CDS (7)..(678)
ccggtgatggcggctggtgatggggacgtgaagctaggcaccctggggagtggcagcgagagcagcaacgacggcggcagcgagagtccaggcgacgcgggagcggcagcggaagggggaggctgggcggcggcggcgttggcgcttctgacggggggcggggaaatgctgctgaacgtggcgctggtggctctggtgctgctgggggcctaccggctgtgggtgcgctgggggcggcggggtctgggggccggggccggggcgggcgaggagagccccgccacctctctgcctcgcatgaagaagcgggacttcagcttggagcagctgcgccagtacgacggctcccgcaacccgcgcatcctgctcgcggtcaatgggaaagtcttcgacgtgaccaaaggcagcaagttctacggcccggcgggtccatatggaatatttgctggtagggatgcctccagaggactggccacattttgcctagataaagatgcacttagagatgaatatgatgatctctcagatttgaatgcagtacaaatggagagtgttcgagaatgggaaatgcagtttaaagaaaaatatgattatgtaggcagactcctaaaaccaggagaagaaccatcagaatatacagatgaagaagataccaaggatcacaataaacaggattgaactttgtaaacaaccaaagtcaggggccttcagaactgcaattcttactccctttcacagactgtccggagtctttgggtttgattcacctgctgcgaaaaacattcaacaaattgtgtacaagataaattaatctcactatgaagatttgaataactagacattatttatgctgccaaactcatttgttgcagttgtttgtaatgtctagtggggcttcatcatcctgaaaagaaggagacagggatttttttaaagagcaagaaagtcacaatattacttctttccttccttttttccttctttcctttcttctttctctttctttctttttaaaatatattgaagacaaccagatatgtatttgctactcaagtgtacagatctcctcaagaaacatcaagggactcctgtgtcacatactgtgtttttattttaacatgggtgagggaggcgacctgatcaggggaggtgggggtacacatcaatttgagttgttcaggctactgaaacattaaaatgtgaattcccaaacttttctttttggctttgtcagggaaaagaaaaatatctttataaagaaatctttggaaattaggagaaggaatttcaggtgggtttaagtcagagctagttccccaacagaaagatcatttgaaaccagtttttatcccttctctttccttccctttccctaaatcaaatcaatattaattgtgccttatttcacttaacatagacttgaattatttttagggaaagcccctataatgaattcagaaatcactacaagcagcattaagactgaagttggaatattctgttgaccataaaaccttgatatcattctgtgtatatagaatgtaaaaggaatattacagtgttaactgccatatatgtaatatacacaaactcaattagcattgtaatggccaaatgcattcccccatgcttttctgttttcaaaaaaattgaaaaacaaatcaactcttatccccaacagctgcctaattttaggagtctgaccctccacatctcactggtgtgggtgcatggggctgtggagtgggtgtcagtatggatgtgtctgaatgtgtgaggccttggaagggactctttctgcagatactgtaaatacaagtaccattttaataaagcatgtacaataaaccaaaaaaaaaaaaaaaaaaaaaaaa

SEQ ID NO: 42 - protein, 223 aa, Homo sapiens
MAAGDGDVKLGTLGSGSESSNDGGSESPGDAGAAAEGGGWAAAALALLTGGGEMLLNVALVALVLLGAYRLWVRWGRRGLGAGAGAGEESPATSLPRMKKRDFSLEQLRQYDGSRNPRILLAVNGKVFDVTKGSKFYGPAGPYGIFAGRDASRGLATFCLDKDALRDEYDDLSDLNAVQMESVREWEMQFKEKYDYVGRLLKPGEEPSEYTDEEDTKDHNKQD

SEQ ID NO: 43 - DNA, 3795 bp, Homo sapiens
  CDS (236)..(1633)
cagcggcggtggcccaggtagaggggtccgcgctggcggcggcggcggcgctgttccccgcgcggtccgcggagcggggtccgggctgcgcgacgtggggcggcggcggcactgcggccccggcccaagcccgaccccgggtcccctcctcggccgccccccgcccggccgcccgccctcgggcctccccccgggccctcggtcccctcccccgctggcggggcccggacagaagatggtgcagaagaaaccagccgaacttcagggtttccaccgttcgttcaaggggcagaaccccttcgagctggccttctccctagaccagcccgaccacggagactctgactttggcctgcagtgctcagcccgccctgacatgcccgccagccagcccattgacatcccggacgccaagaagaggggcaagaagaagaagcgcggccgggccaccgacagcttctcgggcaggtttgaagacgtctaccagctgcaggaagatgtgctgggggagggcgctcatgcccgagtgcagacctgcatcaacctgatcaccagccaggagtacgccgtcaagatcattgagaagcagccaggccacattcggagcagggttttcagggaggtggagatgctgtaccagtgccagggacacaggaacgtcctagagctgattgagttcttcgaggaggaggaccgcttctacctggtgtttgagaagatgcggggaggctccatcctgagccacatccacaagcgccggcacttcaacgagctggaggccagcgtggtggtgcaggacgtggccagcgccttggactttctgcataacaaaggcatcgcccacagggacctaaagccggaaaacatcctctgtgagcaccccaaccaggtctcccccgtgaagatctgtgacttcgacctgggcagcggcatcaaactcaacggggactgctcccctatctccaccccggagctgctcactccgtgcggctcggcggagtacatggccccggaggtagtggaggccttcagcgaggaggctagcatctacgacaagcgctgcgacctgtggagcctgggcgtcatcttgtatatcctactcagcggctacccgcccttcgtgggccgctgtggcagcgactgcggctgggaccgcggcgaggcctgccctgcctgccagaacatgctgtttgagagcatccaggagggcaagtacgagttccccgacaaggactgggcccacatctcctgcgctgccaaagacctcatctccaagctgctggtccgtgacgccaagcagaggctgagtgccgcccaagtcctgcagcacccctgggttcaggggtgcgccccggagaacaccttgcccactcccatggtcctgcagaggaacagctgtgccaaagacctcacgtccttcgcggctgaggccattgccatgaaccggcagctggcccagcacgacgaggacctggctgaggaggaggccgcggggcagggccagcccgtcctggtccgagctacctcacgctgcctgcagctgtctccaccctcccagtccaagctggcgcagcggcggcaaagggccagtctgtcctcggccccagtggtcctggtgggagaccacgcctgaccctcccatctcccctctgtacataggtcacccgtcccccaatcaaatctaaaggttttttaagctatcgccagccggtgtccagcgggctgcccctcctctgcctggattcccaggcactaagctcagctgaggggggtgttttatagaaggtttttgcttttgggttttttttttcctgtttccacccctccccgttattttttcctttggatggttaaaagcattgcaggcacccgggaaggtgagcagagggtaggtgggtgggcttgtcccctccccggtcccccgccctgctcacctctactatgaaggtgcccccaggtcacctgtgctgcccgccatctgcccacgtggcttgcagtgactcaggagagcaggcccacagcgtttgccatcttgcagagctggggaggggcacaggaccctgccctcgtgttccctcccagcccgcagtatttcagggacaggctcttcccctctatccctcaccctgagagcacccctggtggcttggttggggaagggaggggctgcctgtctctggaggtgtcaggcaggcaggtggcaggcagctcacccacccaccccatgggatcccccagcccttcacccgcgcctgccttgtccccatgatagttgacaatcggggcttcctgcaaggcccgtctgtctgtccaggactcctggtggccagattcggcctccgaccttgaccttaaactgcagctgaccccaggggctcgccgctgcccctcccctccacaccaaggcctgagacagcaggagccccgcctggcccgaagccgtttccaccgcagcaggcagaggggctggacaggcactgtcagccaatgtggggggtcctgaagacacccccttggggcacccgagtgccccttctcagggctcagtctgaccgtagccacgtcctgcctcgcgccgcccctcgggcctgacctggaagctccgtcagctccgtccttgtccttagagctgagcccagaccccggggtctggccgaatcctcacccccagggcagtgtttttggtctgccaccttcaggaaaacggctgcggcctcggcctcccttcgggcacccaggaatgcgggggtctgctcagtccccccaccctccatgctccaacccccgggggctgcggagcctgctgccccctccccgcgggtggggacgttctatgcaatacagggttccactttagaagtgcgcgcggctagggtcaccgcccgcccttcccggcgcagcccccgagctccacagctggggcagcccctctggcttctaaatccgcggtcgggattcttcctcctgtttagttttttagtttttccttaaaaaaaaacaacacatcgatggactttgcttccctgttcttgaagaatacttgaatgtcggggggcctgggggtgggggcctcggagaccgtctgcctggccctgctgcccctcctgaatctcgtatgatggtcacagtccggtggccgtgggggtgctctgccttccctggtccccactgcccatatctgtggactgccccttccaaagacccctggggggggtggggcattccgcccacccctttcccccatcacttctcgcctgtcagtgattccatgtttcgtaacgggggattctctgcctttttgtatcaaagaacaagcaaatggacccccgcccgctgcaggcgcccatagccatcgggtctctaaagctgagtggctagcagcgtttgtttgtttgtttttttttttttttctgaaggtgggacagtcacttcctcctccctccccacccctgtcgcatccacgtgcgacctggaggactggtcagaaccgttactgtgaatgagtgaagatcctggaggaccctgggccccaggccagctcccatcgctgggggacggtgaacggccatgtgttaatgttacgatgtttttaaaagacaaaaaaaaaaaaaaaacctcaaaagtttttttaaagtgggggaaaaacatccaagcactttaattccaatgtaccaggtgaactgacggagctcagaagttttcctttacaccaactgtcaatgccggaattttgtattctgttttgtaaagatttaataaaagtcaaaaaacttgcaaaaaaaaaaaaaaaaaaaa

SEQ ID NO: 44 - protein, 465 aa, Homo sapiens
MVQKKPAELQGFHRSFKGQNPFELAFSLDQPDHGDSDFGLQCSARPDMPASQPIDIPDAKKRGKKKKRGRATDSFSGRFEDVYQLQEDVLGEGAHARVQTCINLITSQEYAVKIIEKQPGHIRSRVFREVEMLYQCQGHRNVLELIEFFEEEDRFYLVFEKMRGGSILSHIHKRRHFNELEASVVVQDVASALDFLHNKGIAHRDLKPENILCEHPNQVSPVKICDFDLGSGIKLNGDCSPISTPELLTPCGSAEYMAPEVVEAFSEEASIYDKRCDLWSLGVILYILLSGYPPFVGRCGSDCGWDRGEACPACQNMLFESIQEGKYEFPDKDWAHISCAAKDLISKLLVRDAKQRLSAAQVLQHPWVQGCAPENTLPTPMVLQRNSCAKDLTSFAAEAIAMNRQLAQHDEDLAEEEAAGQGQPVLVRATSRCLQLSPPSQSKLAQRRQRASLSSAPVVLVGDHA

SEQ ID NO: 45 - DNA, 631 bp, Homo sapiens
gaggtgaggggcgcctctgcccggccgcccctactgggaagtgaggagcccctctgcctggccagccgccccgtccgggagggcggtgggggggtcagcccccctcccggccagccgccccatctgggaggtgaggggcacttctgccgggccgcccctactgggaagtgaggagcccctctgcccggccacgaccccgtctgggaggtgtgcccagcggctcattggggatgggccatgatgacaatggcggttttgtggaatagaaaggcgggaagggtggggaaaaaattgagaaatcggatggttgcggggtctgtgtggatagaagtagacatgggagacttttcattttgttctgtactaagaaaaattcttctgccttgggatcctgttgatctgtgaccttatccccaaccctgtgctctctgaaacatgtgctgtgtccactcagggttaaatggattaagggcggtgcaagatgtgctttgttaaacagatgcttgaaggcagcatgctcgttaagagtcatcaccactccctaatcttaagtacccagggacacaaacactgcggaaggccgcagggtcctctgcctaggaaaaccagagacctttgttcacttgttatc

SEQ ID NO: 46 - DNA, 472 bp, Homo sapiens
tactgggaagtgaggaacccctctgcccggccagccgccccgtccgggagggaggtggggggatcagccccccgcctggcagccgccccgtccgggaggtgaggggcgcctctgcccggccgcccctactgggaagtgaggagcccctctgcccggccagccgccccgtccgggagggaggtgggggggtcagccgccgcccgtccgggacctagctgggcaccgatgggccggcacgggtacggtggcaggaagcgtcaggcgagcgaagtgcgcgtaacagtgccagcgacgacgagacgagcaaaggtagtgccgagtgccagtacatgatgccatcctcagacgatgggaggcggtgtcgacgacccgcgcgctgtactgagcaacagggtacactaagaatacaacagtgggtcgcagtgcctatgagagaataagtgatgacgcgtgtcgagcgattgtacccaga

SEQ ID NO: 47 - DNA, 7026 bp, Homo sapiens
  CDS (420)..(5045)
gacacgcacgcacccgggcgccgaagggaaagccgcgtctcgccctcccgccccgccgtcggtcctgtctcagtccctcagcagagcgggaaagcggaggccggagccgtgacctctgaccccgtggttatgcggagccgccgcattccttagcgatcgcggggcagccgccgctgccgccgtgggcgactgacgcagcgcgggcgcgtggagccgccgccgcccctcccccaccgccgctctcgcgccagccggtccccgcgtgcccgccccttctccccggccgcacccgagacctcgcgcgccgccgctgccacgcgccccccccaccgccgccgccgccccagccccgcgccaccgccccagcccgcccagcccggaggtcccgcgtggagctgccgccgccgccggggagaaggatgaaggacaaacagaagaagaagaaggagcgcacgtgggccgaggccgcgcgcctggtattagaaaactactcggatgctccaatgacaccaaaacagattctgcaggtcatagaggcagaaggactaaaggaaatgagaagtgggacttcccctctcgcatgcctcaatgctatgctacattccaattcaagaggaggagaggggttgttttataaactgcctggccgaatcagccttttcacgctcaagaaggatgccctgcagtggtctcgccatccagctacagtggagggagaggagccagaggacacggctgatgtggagagctgtgggtctaatgaagccagcactgtgagtggtgaaaacgatgtatctcttgatgaaacatcttcgaacgcatcctgttctacagaatctcagagtcgacctctttccaatcccagggacagctacagagcttcctcacaggcgaacaaacaaaagaaaaagactggggtgatgctgcctcgagttgtcctgactcctctgaaggtaaacggggcccacgtggaatctgcatcagggttctcgggctgccacgccgatggcgagagcggcagcccgtccagcagcagcagcggctctctggccctgggcagcgctgctattcgtggccaggccgaggtcacccaggaccctgccccgctcctgagaggcttccggaagccagccacaggtcaaatgaagcgcaacagaggggaagaaatagattttgagacacctgggtccattcttgtcaacaccaacctccgtgccctgatcaactctcggaccttccatgccttaccatcacacttccagcagcagctcctcttcctcctgcctgaagtagacagacaggtggggacggatggcctgttgcgtctcagcagcagtgcactaaataacgagttttttacccatgcggctcagagctggcgggagcgcctggctgatggtgaatttactcatgagatgcaagtcaggatacgacaggaaatggagaaggaaaagaaggtggaacaatggaaagaaaagttctttgaagactactatggacagaagctgggtttgaccaaagaagagtcattgcagcagaacgtgggccaggaggaggctgaaatcaaaagtggcttgtgtgtcccaggagaatcagtgcgtatacagcgtggtccagccacccgacagcgagatgggcattttaagaaacgctctcggccagatctccgaaccagagccagaaggaatctgtacaaaaaacaggagtcagaacaagcaggggttgctaaggatgcaaaatctgtggcctcagatgttcccctctacaaggatggggaggctaagactgacccagcagggctgagcagtccccatctgccaggcacatcctctgcagcacccgacctggagggtcccgaattcccagttgagtctgtggcttctcggatccaggctgagccagacaacttggcacgtgcctctgcatctccagacagaattcctagcctgcctcaggaaactgtggatcaggaacccaaggatcagaagaggaaatcctttgagcaggcggcctctgcatcctttcccgaaaagaagccccggcttgaagatcgtcagtcctttcgtaacacaattgaaagtgttcacaccgaaaagccacagcccactaaagaggagcccaaagtcccgcccatccggattcaactttcacgtatcaaaccaccctgggtggttaaaggtcagcccacttaccagatatgcccccggatcatccccaccacggagtcctcctgccggggttggactggcgccaggaccctcgcagacattaaagcccgtgctctgcaggtccgaggggcgagaggtcaccactgccatagagaggcggccaccactgccatcggaggggggggtggcccgggtggaggtggcggcggggccaccgatgagggaggtggcagaggcagcagcagtggtgatggtggtgaggcctgtggccaccctgagcccaggggaggcccgagcacccctggaaagtgtacgtcagatctacagcgaacacaactactgccgccttatcctctaaatggggagcatacccaggccggaactgccatgtccagagctaggagagaggacctgccttctctgagaaaggaggaaagctgcctactacagagggctacagttggactcacagatgggctaggagatgcctcccaactccccgttgctcccactggggaccagccatgccaggccttgcccctactgtcctcccaaacctcagtagctgagagattagtggagcagcctcagttgcatccggatgttagaactgaatgtgagtctggcaccacttcctgggaaagtgatgatgaggagcaaggacccaccgttcctgcagacaatggtcccattccgtctctagtgggagatgatacattagagaaaggaactggccaagctcttgacagtcatcccactatgaaggatcctgtaaatgtgacccccagttccacacctgaatcctcaccgactgattgcctgcagaacagagcatttgatgacgaattagggcttggtggctcatgccctcctatgagggaaagtgatactagacaagaaaacttgaaaaccaaggctctcgtttctaacagttctttgcattggatacccatcccatcgaatgatgaggtagtgaaacagcccaaaccagaatccagagaacacataccatctgttgagccccaggttggagaggagtgggagaaagctgctcccacccctcctgcattgcctggggatttgacagctgaggagggtctagatcctcttgacagccttacttcactctggactgtgccatctcgaggaggcagtgacagcaatggcagttactgtcaacaggtggacattgaaaagctgaaaatcaacggagactctgaagcactgagtcctcacggtgagtccacggatacagcctctgactttgaaggtcacctcacggaggacagcagtgaggctgacactagagaagctgcagtgacaaagggatcttcggtggacaaggatgagaaacccaattggaaccaatctgccccactgtccaaggtgaatggtgacatgcgtctggttacaaggacagatgggatggttgctcctcagagctgggtgtctcgagtatgtgcggtccgccaaaagatcccagattccctactgctggccagtactgagtaccagccaagagccgtgtgcctgtccatgcctgggtcctcagtggaggccactaacccacttgtgatgcagttgctgcagggtagcttgcccctagagaaggttcttccaccagcccacgatgacagcatgtcagaatccccacaagtaccacttacaaaagaccagagccatggctcgctacgcatgggatctttacatggtcttggaaaaaacagtggcatggttgatggaagcagccccagttctttaagggctttgaaggagcctcttctgccagatagctgtgaaacaggcactggtcttgccaggattgaggccacccaggctcctggagcaccccaaaagaattgcaaggcagtcccaagttttgactccctccatccagtgacaaatcccattacatcctctaggaaactggaagaaatggattccaaagagcagttctcttcctttagttgtgaagatcagaaggaagtccgtgctatgtcacaggacagtaattcaaatgctgctccaggaaagagcccaggagatcttactacctcgagaacacctcgtttctcatctccaaatgtgatctcctttggtccagagcagacaggtcgggccctgggtgatcagagcaatgttacaggccaagggaagaagctttttggctctgggaatgtggctgcaacccttcagcgccccaggcctgcggacccgatgcctcttcctgctgagatccctccagtttttcccagtgggaagttgggaccaagcacaaactccatgtctggtggggtacagactccaagggaagactgggctccaaagccacatgcctttgttggcagcgtcaagaatgagaagacttttgtggggggtcctcttaaggcaaatgccgagaacaggaaagctactgggcatagtcccctggaactggtgggtcacttggaagggatgccctttgtcatggacttgcccttctggaaattaccccgagagccagggaaggggctcagtgagcctctggagccttcttctctcccctcccaactcagcatcaagcaggcattttatgggaagctttctaaactccaactgagttccaccagctttaattattcctctagctctcccacctttcccaaaggccttgctggaagtgtggtgcagctgagccacaaagcaaactttggtgcgagccacagtgcatcactttccttgcaaatgttcactgacagcagcacggtggaaagcatctcgctccagtgtgcgtgcagcctgaaagccatgatcatgtgccaaggctgcggtgcgttctgtcacgatgactgtattggaccctcaaagctctgtgtattgtgccttgtggtgagataataaattatggccatgggaaacattgtatatttagtgtgtgtattttgataatgattgatcttaaatctgtatacagaatatcattgatataatactctttaggcaggagcactcttgccttcccccaaaatttacactgctaaagccctctgtcacttggcgacccttctggtcttgctggaggggtttcctgggtataacccattgggctgcccaaggccagccagcctgagctctcctgcaagacagagcctgatgtggcacggagtggggttgcggggggtggggggactgcctgactcccagagggacttgaaactgaagcaagaaggttgcattctccaccaagggagttaacctacctgaactaagtagaaatgccagtcttccactaccccctccctgccatcttttcttctgctactttggggagttgatggccaggaaagaagccagcacagggttaaagtaactcctggcattgcccaccagggggctggtgcacctgctgacctcagggtcacagttgagtcatttgccagttgacggagcaagtttgaccttggttctgttgctgaagcaaatttggaacttttctgtctcagtgtgatccactaacccacaggatcatttggaaccttgaatagctctgcttggacaatggggttggggaatagggttgtctttcctatgaaaatgccatctgtagaccttgtgagtcagccgtccagatgtttgcaggtgaattcctctgcttgacatcctccctgtcactttggaccctatgggagtgggcatctccacgcacctgtgtatgtgaaagtcattttacatttcaaagcagtgtgtgtttcttatttttatatttttaactctttattcttggatgtataaagtgaactttttggcttctgtaagtatgctctatgcacctctaatgttttatcatgtatttatatgttgtacacagtactggctgattctgtaaatggatgtattgtacagagaacatgaacgtctcttcctaattttacatcttcagcatcattgcattaaagtggtgtaatctccttctctacatctgttgtcagagccactgagtgctgtgctgctcgacgtgagggtgaaatgattgacttgtgacctgccaggttgcccgatgccctgttgggtcaccggctggacctgctgcagcctgcagagccacagtcagcctgcccacatgccaccgagcaaacgcatcttgcttttcacatctctcctcctacagccttaatggctgcttgctgccatatgtgacaaatcaccaccaccagtgttaagtgcttctggattcatgggtgagttccctgggcagcccccaggaaggccttccagatctggctccagggtcaccacctgtcacagcaatacctgggaccatgctctcctgggactgtgaggctccttttgacgtacttttgacatcaggcaggtttgggaagaaacaaagccatgcctgctcctgcctctctcccaacatgtttccagcaagtagatgcccctgtgtgtgttttcccttgccttgtttcctgccttatatcttgtatttcgacttattacagagttgagggttcttgcttaatttagatcaagtataaaatttgtatgacttcaagtctcattttatctgaaaggtttttttctcatttaatctgatgtggcattttcgtcatctgaagcatgagtgacaagttgggaatgatgtggtgatttagaatgcagtattggccaagtccaagttgtcaacttaagcgtctgtttaccaaagaccgggaacaggggcccaaacatgtccagtcctcttcttccctctgctggaacctttggggacactcaagggtacagtttgacactgatctggtccatgaggctgcccagagaaagcactgcttctgtatgtctcttgtggtattggaacaataaacccgtacaacctgca

SEQ ID NO: 48 - protein, 1541 aa, Homo sapiens
MKDKQKKKKERTWAEAARLVLENYSDAPMTPKQILQVIEAEGLKEMRSGTSPLACLNAMLHSNSRGGEGLFYKLPGRISLFTLKKDALQWSRHPATVEGEEPEDTADVESCGSNEASTVSGENDVSLDETSSNASCSTESQSRPLSNPRDSYRASSQANKQKKKTGVMLPRVVLTPLKVNGAHVESASGFSGCHADGESGSPSSSSSGSLALGSAAIRGQAEVTQDPAPLLRGFRKPATGQMKRNRGEEIDFETPGSILVNTNLRALINSRTFHALPSHFQQQLLFLLPEVDRQVGTDGLLRLSSSALNNEFFTHAAQSWRERLADGEFTHEMQVRIRQEMEKEKKVEQWKEKFFEDYYGQKLGLTKEESLQQNVGQEEAEIKSGLCVPGESVRIQRGPATRQRDGHFKKRSRPDLRTRARRNLYKKQESEQAGVAKDAKSVASDVPLYKDGEAKTDPAGLSSPHLPGTSSAAPDLEGPEFPVESVASRIQAEPDNLARASASPDRIPSLPQETVDQEPKDQKRKSFEQAASASFPEKKPRLEDRQSFRNTIESVHTEKPQPTKEEPKVPPIRIQLSRIKPPWVVKGQPTYQICPRIIPTTESSCRGWTGARTLADIKARALQVRGARGHHCHREAATTAIGGGGGPGGGGGGATDEGGGRGSSSGDGGEACGHPEPRGGPSTPGKCTSDLQRTQLLPPYPLNGEHTQAGTAMSRARREDLPSLRKEESCLLQRATVGLTDGLGDASQLPVAPTGDQPCQALPLLSSQTSVAERLVEQPQLHPDVRTECESGTTSWESDDEEQGPTVPADNGPIPSLVGDDTLEKGTGQALDSHPTMKDPVNVTPSSTPESSPTDCLQNRAFDDELGLGGSCPPMRESDTRQENLKTKALVSNSSLHWIPIPSNDEVVKQPKPESREHIPSVEPQVGEEWEKAAPTPPALPGDLTAEEGLDPLDSLTSLWTVPSRGGSDSNGSYCQQVDIEKLKINGDSEALSPHGESTDTASDFEGHLTEDSSEADTREAAVTKGSSVDKDEKPNWNQSAPLSKVNGDMRLVTRTDGMVAPQSWVSRVCAVRQKIPDSLLLASTEYQPRAVCLSMPGSSVEATNPLVMQLLQGSLPLEKVLPPAHDDSMSESPQVPLTKDQSHGSLRMGSLHGLGKNSGMVDGSSPSSLRALKEPLLPDSCETGTGLARIEATQAPGAPQKNCKAVPSFDSLHPVTNPITSSRKLEEMDSKEQFSSFSCEDQKEVRAMSQDSNSNAAPGKSPGDLTTSRTPRFSSPNVISFGPEQTGRALGDQSNVTGQGKKLFGSGNVAATLQRPRPADPMPLPAEIPPVFPSGKLGPSTNSMSGGVQTPREDWAPKPHAFVGSVKNEKTFVGGPLKANAENRKATGHSPLELVGHLEGMPFVMDLPFWKLPREPGKGLSEPLEPSSLPSQLSIKQAFYGKLSKLQLSSTSFNYSSSSPTFPKGLAGSVVQLSHKANFGASHSASLSLQMFTDSSTVESISLQCACSLKAMIMCQGCGAFCHDDCIGPSKLCVLCLVVR

SEQ ID NO: 49 - DNA, 3124 bp, Homo sapiens
  CDS (313)..(1731)
taagatccacatcagctcaactgcacttgcctcgcagaggcagcccgctcacttcccgcggaggcgctccccggcgccgcgctccgcggcagccgcctgcccccggcgctgcccccgcccgccgcgccgccgccgccgccgcgcacgccgcgccccgcagctctgggcttcctcttcgcccgggtggcgttgggcccgcgcgggcgctcgggtgactgcagctgctcagctcccctcccccgccccgcgccgcgcggccgcccgtcgcttcgcacagggctggatggttgtattgggcagggtggctccaggatgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctagctcccgggactggggggtttgtctggcatagccatgctggtagcaagagagaaaaaatcaacagcaaacaaaaccacacaaaccaaaccgtcaacagcataataaaatcccaacaactatttttatttcatttttcatgcacaacctttcccccagtgcaaaagactgttactttattattgtattcaaaattcattgtgtatattactacaaagacaaccccaaaccaatttttttcctgcgaagtttaatgatccacaagtgtatatatgaaattctcctccttccttgcccccctctctttcttccctctttcccctccagacattctagtttgtggagggttatttaaaaaaacaaaaaaggaagatggtcaagtttgtaaaatatttgtttgtgctttttccccctccttacctgaccccctacgagtttacaggtctgtggcaatactcttaaccataagaattgaaatggtgaagaaacaagtatacactagaggctcttaaaagtattgaaagacaatactgctgttatatagcaagacataaacagattataaacatcagagccatttgcttctcagtttacatttctgatacatgcagatagcagatgtctttaaatgaaatacatgtatattgtgtatggacttaattatgcacatgctcagatgtgtagacatcctccgtatatttacataacatatagaggtaatagataggtgatatacatgatacattctcaagagttgcttgaccgaaagttacaaggaccccaacccctttgtcctctctacccacagatggccctgggaatcaattcctcaggaattgccctcaagaactctgcttcttgctttgcagagtgccatggtcatgtcattctgaggtcacataacacataaaattagtttctatgagtgtataccatttaaagaatttttttttcagtaaaagggaatattacaatgttggaggagagataagttatagggagctggatttcaaaacgtggtccaagattcaaaaatcctattgatagtggccattttaatcattgccatcgtgtgcttgtttcatccagtgttatgcactttccacagttggacatggtgttagtatagccagacgggtttcattattatttctctttgctttctcaatgttaatttattgcatggtttattctttttctttacagctgaaattgctttaaatgatggttaaaattacaaattaaattgttaatttttatcaatgtgattgtaattaaaaatattttgatttaaataacaaaaataataccagattttaagccgtggaaaatgttcttgatcatttgcagttaaggactttaaataaatcaaatgttaacaaaaaaaaaaaaaaaa

SEQ ID NO: 50 - protein, 472 aa, Homo sapiens
MLGTVKMEGHETSDWNSYYADTQEAYSSVPVSNMNSGLGSMNSMNTYMTMNTMTTSGNMTPASFNMSYANPGLGAGLSPGAVAGMPGGSAGAMNSMTAAGVTAMGTALSPSGMGAMGAQQAASMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 51 - DNA, 3780 bp, Homo sapiens
  CDS (960)..(2282)
agagcgctgccgccgccgctttcgcccgggagccgggggccgggcgccatcatgctgagccggctcggggcgctgctgcaggaagccgtgggggcgcgcgagcccagcattgacctgctgcaggccttcgtggagcactggaagggcatcacgcactactacatcgagagcacagatgaaagcacccccgccaagaagacagacattccctggcggctgaagcagatgctggatatcctggtgtatgaagagcagcagcaggcggccgcgggtgaggcagggccctgcctggagtacctgctgcagcacaagatcctggagactctctgcacgctgggcaaggccgagtaccccccaggcatgcggcagcaggtgttccagttcttcagcaaggttctggcgcaggtgcagcaccccctgctgcattacctcagcgtccacaggcctgtgcagaaactcctccgacttggtgggactgcttccggatccgttacagaaaaggaggaggtgcagttcaccaccgtcctctgctccaagatccagcaggacccagagctgctcgcctacatcctggaaggtaaaaagattgtaggtaggaagaaagcatgcggagaacccactgccctgcctaaggacacaaccagccacggggacaaggactgctcccacgatggtgctcctgccaggccccagctggacggggagtcctgtggggcccaggccttgaacagccacatgcctgctgagaccgaggagctggacggtgggaccacagagagcaacctgattacctccctgcttgggctgtgccagagcaagaagagtcgggtggccttgaaggcccaggagaacctgctgctcctggtgagcatggcctccccagcagctgccacctacctggtacagagcagcgcctgctgccctgcgatcgtccggcactttgccagttgtaccggtccatgcctgtcttcctggaccccgcagacattgccaccttagagggcatcagctggaggttacccagtgccccgtctgatgaggcttccttccctggcaaggaggccttggctgccttcttgggctggtttgattactgcgaccacctcatcacagaggcacacacggtggttgcggacgccttggcgaaggctgtggctgagaacttcttcgtggagaccctgcagccccagctcctgcacgtgtccgagcagagcatcttgacctccaccgccctcctcacagccatgctgcgccagcttcgctcccctgcgctgctgcgggaggccgtggctttcctcctgggcacagaccggcagcctgaagcccccggggacaacccccacaccctgtatgctcatctcatcgggcattgtgaccacctctctgatgagatcagcatcaccacactccggctgtttgaggagctgctgcagaagccccacgaggggatcatccacagcctggtcctgcgcaaccttgagggccgcccttacgtggcctggggctcaccagagcctgagagctatgaggacaccctagacctggaggaagacccctacttcaccgacagcttcctggattccggctttcaaactcccgcaaagcctcgcctagctcctgctaccagttacgatggcaaaacagcagtgaccgagatcgtcaacagtttcctgtgcctggtccccgaggaagccaagacctctgccttcctggaggagacaggctatgacacatacgtccacgatgcttatggcctgttccaggagtgcagctcccgcgtcgcctcctggggctggcctctgacccccacacctttggacccccatgagcccgagcgacctttcttcgagggccacttcctccgagtgctgtttgaccgcatgtcccggattctggatcagccatacagcctgaacctgcaggtgacctcggtcctgtcccggcttgccctcttcccccacccccatattcatgagtacctgctggatccgtacatcagcctggcccccggctgcaggagcctattctccgtgttggtgagggtgatcggggacttgatgcagagaatccagagggtaccccagttcccaggcaagctgctcctggtgcgcaagcagttgacgggccaggctcctggggagcagctggaccaccagaccctcctccagggcgtggtggtgctggaggagttctgcaaggagctggctgccattgccttcgtcaagtttcccccacatgatcctcgccagaacgtctccccagccccggaagggcaggtctgagccagcaccagggcggtgggagactcctgtccacacctctgccccagagctgcctcctgcctggcactgccgccacactcccctcctgggatggggcttctgctcccgggctcactcaaggagactgcggcatgttgaccacaccagactgggtttcagggaatgggcatgccaggtgccaaggagccaaacagatggctttccaggcagcaaggtccttggggccttcttggaggagcttgggtgacagccaggtgagcacccagaccccagaccctcatgtgctgtgtgcctggccccttctgtactggccatttgtggccagggccaagcctgtgactcaactccaggggcaagatggggagtgagctgatggctccgagactggtcaggagcccaggccagtgagatggggcctggagccttgtctgtgtcacattaggtaccatgggagctgctgagacctgacattttgtcccctgcctacatggcttggcccatggagaaggagcagtgaatgggatcgtcggggaagcccctcttcctgctctgctcccctggaaactgttgcaaaactcccagccgcctcatggcaaatgcccaaagcatgttccgcacccaggcgggggcccctgctaatgagaaccttggtgcagctgcagccaggaggggagcgggcccaggagccaggctcaggtccagctggttcctctctggcgccttctgaacccgtctcagcaggtccacagcacctgggcagaggtcagagaccaggggaggccgggccttgccctcccttctgcccagggcccagtgttcttgatagaagacccttctggggagccagggagctcaggggacagataagggaaggacgccccctgactccaggcccctgagcctggcgggaagtggctgcggcccaggcagccagtcctggtggtgttctccctgcatgccctccgtggctgggctgccaccccacccggcccgaatctgtcttgacctgcaggaatacacgggcggcgccaggcattacctcacagcgggactacacagttgctggctttgctcctgggcaaggaggagcaggccagagcctcttttgcttccttttcttgcccatgccgcttctagaagccaggcacaggttgccaagaggtgacacgaaacaggaggaaactcagtgacctctgcctctcccacattcctccccgcgggggaggacctcgccgctctgaagagcaccgtgcacatgtgggtgcacaaacgtgggtgttggtgtggacggggcgcagatctccgtggatgaactgcgtctggactcttagattcataaaatattcgagggtttgggagtcacagaccctcccctctcctcagtgcactttggcatttgcacggtgtcttccccggacagcacagcaataaatggtgtgattgcgtggaaaaaaaaaaaaaaaaaaaaaaaaaaaaaaaaaaaaaaaaaaaaaaaaaa

SEQ ID NO: 52 - protein, 440 aa, Homo sapiens
MPVFLDPADIATLEGISWRLPSAPSDEASFPGKEALAAFLGWFDYCDHLITEAHTVVADALAKAVAENFFVETLQPQLLHVSEQSILTSTALLTAMLRQLRSPALLREAVAFLLGTDRQPEAPGDNPHTLYAHLIGHCDHLSDEISITTLRLFEELLQKPHEGIIHSLVLRNLEGRPYVAWGSPEPESYEDTLDLEEDPYFTDSFLDSGFQTPAKPRLAPATSYDGKTAVTEIVNSFLCLVPEEAKTSAFLEETGYDTYVHDAYGLFQECSSRVASWGWPLTPTPLDPHEPERPFFEGHFLRVLFDRMSRILDQPYSLNLQVTSVLSRLALFPHPHIHEYLLDPYISLAPGCRSLFSVLVRVIGDLMQRIQRVPQFPGKLLLVRKQLTGQAPGEQLDHQTLLQGVVVLEEFCKELAAIAFVKFPPHDPRQNVSPAPEGQV

SEQ ID NO: 53 - DNA, 1175 bp, Homo sapiens
  CDS (68)..(895)
cccggtcgccgcggctgccgcgccgccgctgcaggatagctagcggccaggagaaatacagtggaaaatgcaaaacaacgaaattataaagcctgccaaatacttctcagaattggaaaagagcatcctgctggctttagtagaaaagtataaatatgtgctggaatgtaagaaaagtgatgcgcgaactattgcccttaagcagcgtacctggcaggcgctggcccacgaatacaactctcagcccagcgtgtccctgcgggatttcaaacagctgaagaagtgctgggagaacatcaaggctcggaccaaaaaaattatggcccatgaaaggagagagaaagtgaaacggagcgtcagccctctcctgagtacccacgtcctagggaaggagaagatcgccagcatgctgccggagcagctctacttcctgcagagccccccggaggaggagcccgaataccaccccgacgcctcagcccaagaatcatttgctgtttcaaatagagaactgtgcgatgatgagaaagagttcatacattttccagtatgtgaggggacctctcaacctgaaccctcgtgttcagctgtcagaataacagccaataaaaactacaggagcaaaacctctcaggaaggtgctttaaaaaagatgcatgaggaagaacaccatcaacaaatgtccatcttacaactgcaactgatacaaatgaatgaggtgcatgtggccaaaatccagcagatagagcgagagtgtgagatggcagaggaggaacacaggataaaaatggaagttctcaataaaaagaagatgtattgggaaagaaaactacaaacttttaccaaggaatggcctgtttcctcatttaaccggccctttcccaattcgccctaagactttgggggtggctctcttgtaattaatctgtgttggcaaagaatgtctggaacatggacttggcggtcagtaacctgtaacagagctacaactaggaaaattagagtggtagtagtcacttatttaagaattcattcaggtaaacagctgcaccctctgtaccccttaagtggcaaagaagctgttatagtcttctgaaaattatcactatgagtgctataattctgaatataatgtctcttaattagaattcatacaagaaaaaaaaaaaaaaaaa

SEQ ID NO: 54 - protein, 275 aa, Homo sapiens
MQNNEIIKPAKYFSELEKSILLALVEKYKYVLECKKSDARTIALKQRTWQALAHEYNSQPSVSLRDFKQLKKCWENIKARTKKIMAHERREKVKRSVSPLLSTHVLGKEKIASMLPEQLYFLQSPPEEEPEYHPDASAQESFAVSNRELCDDEKEFIHFPVCEGTSQPEPSCSAVRITANKNYRSKTSQEGALKKMHEEEHHQQMSILQLQLIQMNEVHVAKIQQIERECEMAEEEHRIKMEVLNKKKMYWERKLQTFTKEWPVSSFNRPFPNSP

SEQ ID NO: 55 - DNA, 1988 bp, Homo sapiens
  CDS (12)..(1805)
cctccgggaagatggcggccgtgcaggcggccgaggtgaaagtggatggcagcgagccgaaactgagcaagaatgagctgaagagacgcctgaaagctgagaagaaagtagcagagaaggaggccaaacagaaggagctcagtgagaaacagctaagccaagccactgctgctgccaccaaccacaccactgataatggtgtgggtcctgaggaagagagcgtggacccaaatcaatactacaaaatccgcagtcaagcaattcatcagctgaaggtcaatggggaagacccatacccacacaagttccatgtagacatctcactcactgacttcatccaaaaatatagtcacctgcagcctggggatcacctgactgacatcaccttaaaggtggcaggtaggatccatgccaaaagagcttctgggggaaagctcatcttctatgatcttcgaggagagggggtgaagttgcaagtcatggccaattccagaaattataaatcagaagaagaatttattcatattaataacaaactgcgtcggggagacataattggagttcaggggaatcctggtaaaaccaagaagggtgagctgagcatcattccgtatgagatcacactgctgtctccctgtttgcatatgttacctcatcttcactttggcctcaaagacaaggaaacaaggtatcgccagagatacttggacttgatcctgaatgactttgtgaggcagaaatttatcatccgctctaagatcatcacatatataagaagtttcttagatgagctgggattcctagagattgaaactcccatgatgaacatcatcccagggggagccgtggccaagcctttcatcacttatcacaacgagctggacatgaacttatatatgagaattgctccagaactctatcataagatgcttgtggttggtggcatcgaccgggtttatgaaattggacgccagttccggaatgaggggattgatttgacgcacaatcctgagttcaccacctgtgagttctacatggcctatgcagactatcacgatctcatggaaatcacggagaagatggtttcagggatggtgaagcatattacaggcagttacaaggtcacctaccacccagatggcccagagggccaagcctacgatgttgacttcaccccacccttccggcgaatcaacatggtagaagagcttgagaaagccctggggatgaagctgccagaaacgaacctctttgaaactgaagaaactcgcaaaattcttgatgatatctgtgtggcaaaagctgttgaatgccctccacctcggaccacagccaggctccttgacaagcttgttggggagttcctggaagtgacttgcatcaatcctacattcatctgtgatcacccacagataatgagccctttggctaaatggcaccgctctaaagagggtctgactgagcgctttgagctgtttgtcatgaagaaagagatatgcaatgcgtatactgagctgaatgatcccatgcggcagcggcagctttttgaagaacaggccaaggccaaggctgcaggtgatgatgaggccatgttcatagatgaaaacttctgtactgccctggaatatgggctgccccccacagctggctggggcatgggcattgatcgagtcgccatgtttctcacggactccaacaacatcaaggaagtacttctgtttcctgccatgaaacccgaagacaagaaggagaatgtagcaaccactgatacactggaaagcacaacagttggcacttctgtctagaaaataataattgcaagttgtataactcaggcgtctttgcatttctgcgaaagatcaaggtctgcaagggaattcttgtgtgctgctttccatttgacaccgcagttctgttcagccatcagaagagagacaaggaattaaaaatttctttttaatcctgttaccaaaaaaaaaaaaaaaaaa

SEQ ID NO: 56 - protein, 597 aa, Homo sapiens
MAAVQAAEVKVDGSEPKLSKNELKRRLKAEKKVAEKEAKQKELSEKQLSQATAAATNHTTDNGVGPEEESVDPNQYYKIRSQAIHQLKVNGEDPYPHKFHVDISLTDFIQKYSHLQPGDHLTDITLKVAGRIHAKRASGGKLIFYDLRGEGVKLQVMANSRNYKSEEEFIHINNKLRRGDIIGVQGNPGKTKKGELSIIPYEITLLSPCLHMLPHLHFGLKDKETRYRQRYLDLILNDFVRQKFIIRSKIITYIRSFLDELGFLEIETPMMNIIPGGAVAKPFITYHNELDMNLYMRIAPELYHKMLVVGGIDRVYEIGRQFRNEGIDLTHNPEFTTCEFYMAYADYHDLMEITEKMVSGMVKHITGSYKVTYHPDGPEGQAYDVDFTPPFRRINMVEELEKALGMKLPETNLFETEETRKILDDICVAKAVECPPPRTTARLLDKLVGEFLEVTCINPTFICDHPQIMSPLAKWHRSKEGLTERFELFVMKKEICNAYTELNDPMRQRQLFEEQAKAKAAGDDEAMFIDENFCTALEYGLPPTAGWGMGIDRVAMFLTDSNNIKEVLLFPAMKPEDKKENVATTDTLESTTVGTSV

SEQ ID NO: 57 - DNA, 763 bp, Homo sapiens
ggggctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaacccacaagtcattattaccctcactgtcaacccaa

SEQ ID NO: 58 - DNA, 1575 bp, Homo sapiens
  CDS (196)..(834)
ctcttcccggctccagctccgccgccagctccagcctttgctccccctcccaaagtcccctccccggagcggagcgcacctagggtccctcttccgtccccccagcccagctacccgttcagaccagcagcctcggggggcacccccccgccagcctgcctccctcccgctcagccctgccagggttccccagccatgaatctcttccgattcctgggagacctctcccacctcctcgccatcatcttgctactgctcaaaatctggaagtcccgctcgtgcgccggaatttcagggaagagccaggtcctgtttgctgtggtgttcactgcccgatatctggacctcttcaccaactacatctcactctacaacacgtgtatgaaggtggtctacatagcctgctccttcaccacggtctggttgatttatagcaagttcaaagctacttacgatgggaaccatgacacgttcagagtggagttcctggtcgttcccacagccattctggcgttcctggtcaatcatgacttcacccctctggagatcctctggaccttctccatctacctggagtcagtggccatcttgccgcagctgttcatggtgagcaagaccggcgaggcggagaccatcaccagccactacttgtttgcgctaggcgtttaccgcacgctctatctcttcaactggatctggcgctaccatttcgagggcttcttcgacctcatcgccattgtggcaggcctggtccagacagtcctctactgcgatttcttctacctctatatcaccaaagtcctaaaggggaagaagttgagtttgccggcatagccccggtcctctccatctctctcctcggcagcagcgggaggcagaggaaggcggcagaagatgaagagctttcccatccaggggtgacttttttaagaacccacctcttgtgctccccatcccgcctcctgccgggtttcagggggacagtggaggatccaggtcttggggagctcaggacttgggctgtttgtagttttttgccttttagacaagaaaaaaaaatctttccactctttagtttttgattctgatgactcgtttttcttctactctgtggccccaatttttataaagtgtttttgagtgtcctatgggccggggcagggtccaagatcttttcccttccccaggcccctcggctccctcccagatcccacccccagccccactggttgccaaacactaaatctgccgacacccatctgccccacctcctgccatggccatgaaccgcgacccccactaaatttctagattggggatagggagaaagggaggcccaggaaggtctcccctgattttttttcatagtaatttttttccccagagtttgaattttttggtcttctcctggttttttggcaaattaggggggcccggggctcaagtgcgggaagggggctggcccgaggatcccatggctctcacaccatgtttttgtacagaactgatggttgaatctttgttctcttgaaataaacagaagaaaatgaaacctttaaaaaaaaaaaaaaaaaa

SEQ ID NO: 59 - protein, 212 aa, Homo sapiens
MNLFRFLGDLSHLLAIILLLLKIWKSRSCAGISGKSQVLFAVVFTARYLDLFTNYISLYNTCMKVVYIACSFTTVWLIYSKFKATYDGNHDTFRVEFLVVPTAILAFLVNHDFTPLEILWTFSIYLESVAILPQLFMVSKTGEAETITSHYLFALGVYRTLYLFNWIWRYHFEGFFDLIAIVAGLVQTVLYCDFFYLYITKVLKGKKLSLPA

SEQ ID NO: 60 - DNA, 2245 bp, Homo sapiens
  CDS (157)..(1110)
gaatctcgacccttgaatggagttacacgaacggccagatgaaagaaggaaggcccggacctccactcagggccgactaggggactggcggagggtgcacgctgatggatttactcaccgggtgcttggagctccagcagctggctggagcccgcgatgacgtcacggactcgggtcacatggccgagtccgccccgccccctccccgtccccgccgctgcagccgtcgccttcggagcgaagggtaccgacccggcagaagctcggagctctcggggtatcgaggaggcaggcccgcgggcgcacgggcgagcgggccgggagccggagcggcggaggagccggcagcagcggcgcggcgggctccaggcgaggcggtcgacgctcctgaaaacttgcgcgcgcgctcgcgccactgcgcccggagcgatgaagatggtcgcgccctggacgcggttctactccaacagctgctgcttgtgctgccatgtccgcaccggcaccatcctgctcggcgtctggtatctgatcatcaatgctgtggtactgttgattttattgagtgccctggctgatccggatcagtataacttttcaagttctgaactgggaggtgactttgagttcatggatgatgccaacatgtgcattgccattgcgatttctcttctcatgatcctgatatgtgctatggctacttacggagcgtacaagcaacgcgcagcctggatcatcccattcttctgttaccagatctttgactttgccctgaacatgttggttgcaatcactgtgcttatttatccaaactccattcaggaatacatacggcaactgcctcctaattttccctacagagatgatgtcatgtcagtgaatcctacctgtttggtccttattattcttctgtttattagcattatcttgacttttaagggttacttgattagctgtgtttggaactgctaccgatacatcaatggtaggaactcctctgatgtcctggtttatgttaccagcaatgacactacggtgctgctacccccgtatgatgatgccactgtgaatggtgctgccaaggagccaccgccaccttacgtgtctgcctaagccttcaagtgggcggagctgagggcagcagcttgactttgcagacatctgagcaatagttctgttatttcacttttgccatgagcctctctgagcttgtttgttgctgaaatgctactttttaaaatttagatgttagattgaaaactgtagttttcaacatatgctttgctggaacactgtgatagattaactgtagaattcttcctgtacgattggggatataatgggcttcactaaccttccctaggcattgaaacttcccccaaatctgatggacctagaagtctgcttttgtacctgctgggccccaaagttgggcatttttctctctgttccctctcttttgaaaatgtaaaataaaaccaaaaatagacaactttttcttcagccattccagcatagagaacaaaaccttatggaaacaggaatgtcaattgtgtaatcattgttctaattaggtaaatagaagtccttatgtatgtgttacaagaatttcccccacaacatcctttatgactgaagttcaatgacagtttgtgtttggtggtaaaggattttctccatggcctgaattaagaccattagaaagcaccaggccgtgggagcagtgaccatctgctgactgttcttgtggatcttgtgtccagggacatggggtgacatgcctcgtatgtgttagagggtggaatggatgtgtttggcgctgcatgggatctggtgcccctcttctcctggattcacatccccacccagggcccgcttttactaagtgttctgccctagattggttcaaggaggtcatccaactgactttatcgagtggaattgggatatatttgatatacttctgcctaacaacatggaaaagggttttcttttccctgcaagctacatcctactgctttgaacttccaagtatgtctagtcaccttttaaaatgtaaacattttcagaaaaatgaggattgccttccttgtatgcgctttttaccttgactacctgaattgcaagggatttttatatattcatatgttacaaagtcagcaactctcctgttggttcattattgaatgtgctgtaaattaagttgtttgcaattaaaacaaggtttgcccacaaaaaaaaaaaaaaa

SEQ ID NO: 61 - protein, 317 aa, Homo sapiens
MTSRTRVTWPSPPRPLPVPAAAAVAFGAKGTDPAEARSSRGIEEAGPRAHGRAGREPERRRSRQQRRGGLQARRSTLLKTCARARATAPGAMKMVAPWTRFYSNSCCLCCHVRTGTILLGVWYLIINAVVLLILLSALADPDQYNFSSSELGGDFEFMDDANMCIAIAISLLMILICAMATYGAYKQRAAWIIPFFCYQIFDFALNMLVAITVLIYPNSIQEYIRQLPPNFPYRDDVMSVNPTCLVLIILLFISIILTFKGYLISCVWNCYRYINGRNSSDVLVYVTSNDTTVLLPPYDDATVNGAAKEPPPPYVSA

SEQ ID NO: 62 - DNA, 1453 bp, Homo sapiens
  CDS (65)..(1057)
ggctgccggagggcgggaggcaggagcgggccaggagctgctgggctggagcggcggcgccgccatgtccgacagcgagaagctcaacctggactcgatcatcgggcgcctgctggaagtgcagggctcgcggcctggcaagaatgtacagctgacagagaacgagatccgcggtctgtgcctgaaatcccgggagatttttctgagccagcccattcttctggagctggaggcacccctcaagatctgcggtgacatacacggccagtactacgaccttctgcgactatttgagtatggcggtttccctcccgagagcaactacctctttctgggggactatgtggacaggggcaagcagtccttggagaccatctgcctgctgctggcctataagatcaagtaccccgagaacttcttcctgctccgtgggaaccacgagtgtgccagcatcaaccgcatctatggtttctacgatgagtgcaagagacgctacaacatcaaactgtggaaaaccttcactgactgcttcaactgcctgcccatcgcggccatagtggacgaaaagatcttctgctgccacggaggcctgtccccggacctgcagtctatggagcagattcggcggatcatgcggcccacagatgtgcctgaccagggcctgctgtgtgacctgctgtggtctgaccctgacaaggacgtgcagggctggggcgagaacgaccgtggcgtctcttttacctttggagccgaggtggtggccaagttcctccacaagcacgacttggacctcatctgccgagcacaccaggtggtagaagacggctacgagttctttgccaagcggcagctggtgacacttttctcagctcccaactactgtggcgagtttgacaatgctggcgccatgatgagtgtggacgagaccctcatgtgctctttccagatcctcaagcccgccgacaagaacaaggggaagtacgggcagttcagtggcctgaaccctggaggccgacccatcaccccaccccgcaattccgccaaagccaagaaatagcccccgcacaccaccctgtgccccagatgatggattgattgtacagaaatcatgctgccatgctgggggggggtcaccccgacccctcaggcccacctgtcacggggaacatggagccttggtgtatttttcttttctttttttaatgaatcaatagcagcgtccagtcccccagggctgcttcctgcctgcacctgcggtgactgtgagcaggatcctggggccgaggctgcagctcagggcaacggcaggccaggtcgtgggtctccagccgtgcttggcctcagggctggcagccggatcctggggcaacccatctggtctcttgaataaaggtcaaagctggattctcaaaaaaaaaaaaaaaaaaaaaaaaaaaaaaaaaaaaaaaaaaaa

SEQ ID NO: 63 - protein, 330 aa, Homo sapiens
MSDSEKLNLDSIIGRLLEVQGSRPGKNVQLTENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVQGWGENDRGVSFTFGAEVVAKFLHKHDLDLICRAHQVVEDGYEFFAKRQLVTLFSAPNYCGEFDNAGAMMSVDETLMCSFQILKPADKNKGKYGQFSGLNPGGRPITPPRNSAKAKK

SEQ ID NO: 64 - DNA, 1591 bp, Homo sapiens
  CDS (44)..(814)
ccctgcgtctctgcccgccccgtggcgcccgagtgcactgaagatggcggctgctgtaggacggttgctccgagcgtcggttgcccgacatgtgagtgccattccttggggcatttctgccactgcagccctcaggcctgctgcatgtggaagaacgagcttgacaaatttattgtgttctggttccagtcaagcaaaattattcagcaccagttcctcatgccatgcacctgctgtcacccagcatgcaccctattttaagggtacagccgttgtcaatggagagttcaaagacctaagccttgatgactttaaggggaaatatttggtgcttttcttctatcctttggatttcacctttgtgtgtcctacagaaattgttgcttttagtgacaaagctaacgaatttcacgacgtgaactgtgaagttgtcgcagtctcagtggattcccactttagccatcttgcctggataaatacaccaaggaagaatggtggtttgggccacatgaacatcgcactcttgtcagacttaactaagcagatttcccgagactacggtgtgctgttagaaggttctggtcttgcactaagaggtctcttcataattgaccccaatggagtcatcaagcatttgagcgtcaacgatctcccagtgggccgaagcgtggaagaaaccctccgcttggtgaaggcgttccagtatgtagaaacacatggagaagtctgcccagcgaactggacaccggattctcctacgatcaagccaagtccagctgcttccaaagagtactttcagaaggtaaatcagtagatcacccatgtgtatctgcaccttctcaactgagagaagaaccacagttgaaacctgcttttatcattttcaagatggttatttgtagaaggcaaggaaccaattatgcttgtattcataagtattactctaaatgttttgtttttgtaattctggctaagaccttttaaacatggttagttgctagtacaaggaatcctttattggtaacatcttggtggctggctagctagtttctacagaacataatttgcctctatagaaggctattcttagatcatgtctcaatggaaacactcttctttcttagccttacttgaatcttgcctataataaagtagagcaacacacattgaaagcttctgatcaacggtcctgaaattttcatcttgaatgtctttgtattaaactgaattttcttttaagctaacaaagatcataattttcaatgattagccgtgtaactcctgcaatgaatgtttatgtgattgaagcaaatgtgaatcgtattattttaaaaagtggcagagtgacttaactgatcatgcatgatccctcatccctgaaattgagtttatgtagtcattttacttattttattcattagctaactttgtctatgtatatttctagatattgattagtgtaatcgattataaaggatatttatcaaatccagggattgcattttgaaattataattattttctttgctgaagtattcattgtaaaacatacaaaataaacatattttaaaacatttgcattttaccacca

SEQ ID NO: 65 - protein, 256 aa, Homo sapiens
MAAAVGRLLRASVARHVSAIPWGISATAALRPAACGRTSLTNLLCSGSSQAKLFSTSSSCHAPAVTQHAPYFKGTAVVNGEFKDLSLDDFKGKYLVLFFYPLDFTFVCPTEIVAFSDKANEFHDVNCEVVAVSVDSHFSHLAWINTPRKNGGLGHMNIALLSDLTKQISRDYGVLLEGSGLALRGLFIIDPNGVIKHLSVNDLPVGRSVEETLRLVKAFQYVETHGEVCPANWTPDSPTIKPSPAASKEYFQKVNQ

SEQ ID NO: 66 - DNA, 1224 bp, Homo sapiens
  CDS (70)..(1041)
gcccattgtttttgtaatctctgaggagaagcagcagcaaacatttgctagtcagacaagtgacagggaatggattccaaacaccagtgtgtaaagctaaatgatggccacttcatgcctgtattgggatttggcacctatgcacctccagaggttccgagaagtaaagctttggaggtcacaaaattagcaatagaagctgggttccgccatatagattctgctcatttatacaataatgaggagcaggttggactggccatccgaagcaagattgcagatggcagtgtgaagagagaagacatattctacacttcaaagctttggtccacttttcatcgaccagagttggtccgaccagccttggaaaactcactgaagaaagctcaattggactatgttgacctctatcttattcattctccaatgtctctaaagccaggtgaggaactttcaccaacagatgaaaatggaaaagtaatatttgacatagtggatctctgtaccacctgggaggccatggagaagtgtaaggatgcaggattggccaagtccattggggtgtcaaacttcaaccgcaggcagctggagatgatcctcaacaagccaggactcaagtacaagcctgtctgcaaccaggtagaatgtcatccgtatttcaaccggagtaaattgctagatttctgcaagtcgaaagatattgttctggttgcctatagtgctctgggatctcaacgagacaaacgatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtttttgagttccagttgactgcagaggacatgaaagccatagatggcctagacagaaatctccactattttaacagtgatagttttgctagccaccctaattatccatattcagatgaatattaacatggagggctttgcctgatgtctaccagaagccctgtgtgtggatggtgacgcagaggacgtctctatgccggtgactggacatatcacctctacttaaatccgtcctgtttagcgacttcagtcaactacagctgagtccataggccagaaagacaataaatttttatcattttgaaataa

SEQ ID NO: 67 - protein, 323 aa, Homo sapiens
MDSKHQCVKLNDGHFMPVLGFGTYAPPEVPRSKALEVTKLAIEAGFRHIDSAHLYNNEEQVGLAIRSKIADGSVKREDIFYTSKLWSTFHRPELVRPALENSLKKAQLDYVDLYLIHSPMSLKPGEELSPTDENGKVIFDIVDLCTTWEAMEKCKDAGLAKSIGVSNFNRRQLEMILNKPGLKYKPVCNQVECHPYFNRSKLLDFCKSKDIVLVAYSALGSQRDKRWVDPNSPVLLEDPVLCALAKKHKRTPALIALRYQLQRGVVVLAKSYNEQRIRQNVQVFEFQLTAEDMKAIDGLDRNLHYFNSDSFASHPNYPYSDEY

SEQ ID NO: 68 - DNA, 1715 bp, Homo sapiens
  CDS (37)..(591)
ccgtgctctgcctcctgtgttccagggactttgaacatgtcggggatcgccctcagcagactcgcccaggagaggaaagcatggaggaaagaccacccatttggtttcgtggctgtcccaacaaaaaatcccgatggcacgatgaacctcatgaactgggagtgcgccattccaggaaagaaagggactccgtgggaaggaggcttgtttaaactacggatgcttttcaaagatgattatccatcttcgccaccaaaatgtaaattcgaaccaccattatttcacccgaatgtgtacccttcggggacagtgtgcctgtccatcttagaggaggacaaggactggaggccagccatcacaatcaaacagatcctattaggaatacaggaacttctaaatgaaccaaatatccaagacccagctcaagcagaggcctacacgatttactggttagtagcagccctggccccgctggtggcagctcctccccgtcccagccaaggccgcctggcaggacgggagtggagcacacaggctcaccctagggacagccagggtccgcgcctctgtggggaaggtcggggggcataaaccctgtgggcagcaggcaccgttgcacaccgtctgcggtgggcatggatggcgccggagccgtgtccaggggagggagcaggccaggtgacaggggctgtcttgggccactgccccagtgagttgtggccagctaggaggggaaggaccctgggcgtgggtgccagaggaggccatgctgagaggcctctccaccggctcagcctcccgtggaccccagcctcccgcgccgcccttgctgcttgtccccatcccctctacaacttggttccttctgtgggggggcccagcccagcagggctcagtgacgtgatgctgtcacacggaccctgggcttccaccctgtccctgtgaggtgctgctgtggctagaagggtccccgtgggcctggggtcctgtctgcaccgaggagggcctggtgtgagttaggatgcagacgacagccaaacagtgctggggaaagcagggaggaccctgccctgagcagaggccatgcgcccgcctgtcctgttccctctgcagctgttctgggcaggtccataagaaggtgggttggggcctccgacccccactgctccgcctgcagggttgagaaacccagccccatctcaccacgggtccccacacctctgaggaggtctcagggcccccaatggccagggacccagaaggctcacctgcagccaccccacacatccctgccactgtgggcatcagaggccctctccctccagggtgcatctcctggtcaggaggggggaggcccggttgccttaggcccggcctgcacaggtcgtgaggaaaagcacctggtttcggccgggcgcggtggctcacgcttgtaatcccagcactttgggaggccgaggtgggcgaatcatgcggtcaggagttcgagaccagcctggccaacatggcgaaaccctcgtccctactaaaaatacaaaaaattagttgggcgtagtggtgggtgcctctaatcccagctacctgggaggctgagacaggagaatcacttgaacccgggaggcggaggttgcagtgagccgagatcctgccactgctctccagcctggcgacggaggaatgctgtctcaaaaaaaaaaaaaaaaaaaaaaaaaaaa

SEQ ID NO: 69 - protein, 184 aa, Homo sapiens
MSGIALSRLAQERKAWRKDHPFGFVAVPTKNPDGTMNLMNWECAIPGKKGTPWEGGLFKLRMLFKDDYPSSPPKCKFEPPLFHPNVYPSGTVCLSILEEDKDWRPAITIKQILLGIQELLNEPNIQDPAQAEAYTIYWLVAALAPLVAAPPRPSQGRLAGREWSTQAHPRDSQGPRLCGEGRGA

SEQ ID NO: 70 - DNA, 1327 bp, Homo sapiens
  CDS (100)..(966)
ctcctctccgcgcggggcgggctccgcgccacgtgactccgcggccgggccgggacgcgacgggacgcgctgggaccggcgtcgggggtcgcggggaccatgcagcggaggtgggtcttcgtgctgctcgacgtgctgtgcttactggtcgcctccctgcccttcgctatcctgacgctggtgaacgccccgtacaagcgaggattttactgcggggatgactccatccggtacccctaccgtccagataccatcacccacgggctcatggctggggtcaccatcacggccaccgtcatccttgtctcggccggggaagcctacctggtgtacacagaccggctctattctcgctcggacttcaacaactacgtggctgctgtatacaaggtgctggggaccttcctgtttggggctgccgtgagccagtctctgacagacctggccaagtacatgattgggcgtctgaggcccaacttcctagccgtctgcgaccccgactggagccgggtcaactgctcggtctatgtgcagctggagaaggtgtgcaggggaaaccctgctgatgtcaccgaggccaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctgattacaaacaccactggagcgatgtccttgttggcctcctgcagggggcactggtggctgccctcactgtctgctacatctcagacttcttcaaagcccgacccccacagcactgtctgaaggaggaggagctggaacggaagcccagcctgtcactgacgttgaccctgggcgaggctgaccacaaccactatggatacccgcactcctcctcctgaggccggaccccgcccaggcagggagctgctgtgagtccagctgaggcccacccaggtggtccctccagccctggttaggcactgagggctctggacgggctccaggaaccctgggctgatgggagcagtgagcgggctccgctgccccctgccctgcactggaccaggagtctggagatgcctgggtagccctcagcatttggaggggaacctgttcccgtcggtccccaaatatccccttctttttatggggttaaggaagggaccgagagatcagatagttgctgttttgtaaaatgtaatgtatatgtggtttttagtaaaatagggcacctgtttcacaaaaaaaaaaaaaaaaaaa

SEQ ID NO: 71 - protein, 288 aa, Homo sapiens
MQRRWVFVLLDVLCLLVASLPFAILTLVNAPYKRGFYCGDDSIRYPYRPDTITHGLMAGVTITATVILVSAGEAYLVYTDRLYSRSDFNNYVAAVYKVLGTFLFGAAVSQSLTDLAKYMIGRLRPNFLAVCDPDWSRVNCSVYVQLEKVCRGNPADVTEARLSFYSGHSSFGMYCMVFLALYVQARLCWKWARLLRPTVQFFLVAFALYVGYTRVSDYKHHWSDVLVGLLQGALVAALTVCYISDFFKARPPQHCLKEEELERKPSLSLTLTLGEADHNHYGYPHSSS

SEQ ID NO: 72 - DNA, 2980 bp, Homo sapiens
  CDS (274)..(519)
  misc_feature (2974)..(2974): n = a, t, g or c
ggccgctcctgctgctgctactgccgccgccgcagcggctgctcgggctgagcacgccccggaacaggccgccgcgcgctgcgcgccggacccgctgcccctgccggcccggccgggtcgggcggcccagggaccgacagacttgacaacggtgacagcactggggcggcaccttcctacttctgcccagccacagccctcccctcacagttgagcacctgtttgcctgaagttaatttccagaagcaggagtccccagagccaggcagggggatgaaccgcgagggagctcccgggaagagtccggaggagatgtacattcagcagaaggtccgagtgctgctcatgctgcggaagatgggatcaaacctgacagccagcgaggaggagttcctgcgcacctatgcaggggtggtcaacagccagctcagccagctgcctccgcactccatcgaccagggtgcagaggacgtggtgatggcgttttccaggtcggagacggaagaccggaggcagtagctgcaaagcccttggaacaccctggatgctgttgaagggccaagagatctgtgtggctcctgggccggctgaatggcagcagccccccttgccccacctcccccttccctacccaaccctgccctgccccaccccacctcacagctactcagtggggctggcatcaagggagacaccagtggtgcgtttataattggcttaaagggatggacttgtgattggctgcaggaagaaacttttttattttttaaatcttgaccaacagaaaccttttatttttatttctgactcttattttttaaaaaatttgcgcctcggtatctggcttccctggaagctctccgagctctggtgctttagttaggtcatttttttagaaatgtgaagaggtctgattggctgcttaaactggaaagggactgtgattggctggttaatgggaaacggtttttttctttggctgcaggtgttctgctgatatcaacagcttccctattttgaatgcagaaaacagggtctgggacattagtcgttatatttgacttgaaaagaaagaaaccaagtgcgctttgcaatatttattacacaaagaacttgctgctgccttcacatttggggtttgtgtttgattggctttcgatgcgtgtgtttggtttcccattggttcacctgtgactcctgttgccatggattcacccccctctgctgccggctctgggcctgagggtccacctggagagtacatttgctttaatgagtgcacctgcctccaccagcaaggggaccccgagaaccctgagcagggtccacagctggaaagttgggcccctgaggagctttgtgtcgtcttgaacgagcagcccagggcctagaggtaaccgttaggcgggatttatgtgcactgcctgcatgagctggcaaccagccagcgtcccttggtgagaaagggattgctgaggcaccgtccaggccccaccggccaggccgcgcccagcagaggcgtactacccagctctgtcctcttggccatccttctgtgtaccacttcctgaggcctcattttgggggtcatcttggaaaggggaggagcttctcccagtgtgagaccccaaagactctggaggtcatctggcggaggtctctgggagcccagaacccacataaaagccccagcttggctcacaaggcccaggagacctccagctaaacaccaacccctgacctaccccagccaggctcctacctgtctgctgccagcacagtaggtcccggccagctctggagttctctcatcggaggcccatgccctccactccactgcctttggaagggtctctctccaggtcagcctggaagggacagtatcgtttgtttatgaaatgccactgggacagctggctgggccttcaccaagcaagtcccttcagactggcccttaagccaaactcaggcccagaattgcagttcagaatggcagtcctggaggcagggggtgaggggcaggtctagtgttcctgcaccaaacctaagtccttccacctgccacccccttccctgggagggaggtggtcctcctatctccctggctcactggcaggtgtgggatctggggagagcggctggagaaagatgcagtcctcaggaagggggccgccaccctcccctatgctggtagatgctgaggcccctaggtgcccagggccagtgggaccctctcagaaccaaatctttcccctttctcggggcttggggctcgggccgtaggggctcctgagtgtcatgaagtgcacaggagccaaatgaccgagccctggagagccccatggtgggtaggtggttcgtgctgtgctctggcaccatcagcctgttccagaaggaggattcgagcatcaggctaagaccctgtgtcctccaccatgcactcacccctagccctggttagctgacagtcagctgtggggaacacagctacaaccctaccctggcagggacctgagagcatctcaggaggggcagcgcatgtgtgcatgtgctgtgtgagtgagcacacccgtgtgcacactcatacacatgtgcacacacacgcactctccccgctcaggggcctggaggtctggctgagcccctggggaaaggtgagttctttcatctccctcctccaggtcggagtgcctggagtcaggtgtcgaggccacattgctggctgccccctctttgtagctcctataaagggcccacacctggtggatacctggttgagcgtgtggtctctgccccagcctgtccttgtcacgatcacaggccttgcttttgtaacaatgatgaccccggcctgtctcatcttctgaagaggaaaagtcaaagtgttgctgtggctccatatttcaactaaaaatatatctgttggagaaagaaattaacaataaagaattttcataggttaaaaaaaaaaaanaaaaag

SEQ ID NO: 73 - protein, 81 aa, Homo sapiens
MNREGAPGKSPEEMYIQQKVRVLLMLRKMGSNLTASEEEFLRTYAGVVNSQLSQLPPHSIDQGAEDVVMAFSRSETEDRRQ

SEQ ID NO: 74 - DNA, 2153 bp, Homo sapiens
  CDS (589)..(1356)
cagcggccgggagggggctccggggaccatggggctcctgaccattctgaagaagatgaagcagaaagagcgggagctgcgactgctcatgcttggcctggacaatgctggaaagacaaccatcctgaagaagttcaatggggaggacatcgacaccatctccccaacgctgggcttcaacatcaagaccctggagcaccgaggattcaagctgaacatctgggatgtgggtggccagaagtccctgcggtcctactggcggaactactttgagagcaccgatggcctcatctgggtagtggacagcgcagaccgccagcgcatgcaggactgccagcgggagctccagagcctgctggtggaggagtgacctgggtttacgcttcaccacagagaaggaacttttcagacgctcccttggccgcgcacccgggatcggaacagcgctgtcacttgcgacaaaatccctaggccagaggaagaagagcgcaggcctgggcgaggcggcggcgggcggaggctgggccggaggggtggggacggcgaggaggtggaggccggcgctccgctccgctccagctcggtttcatgtcccgccaggcgaaggatgacttcctgcggcactacacagtgtcggaccccaggactcaccccaagggctacaccgagtacaaagtaaccgcgcagttcatctcaaagaaggacccagaggatgtcaaagaggtggtggtctggaagcggtacagcgacttccgcaagctgcatggagacctggcctacacccaccgcaacctcttccgccgcctcgaggagttccctgctttcccccgggcccaggtgtttggccggtttgaagcctcagtgatcgaggagcggcgaaagggggcagaggacctgcttcgcttcactgtgcacatacctgcgctcaacaacagcccccagctcaaggagttcttccggggtggggaggtgacccgacccttggaggtgtccagggacctacacatcctgccaccccctctgatccccaccccgccccctgatgacccccggctatcccaactgctccctgcagaaaggaggggcctcgaggaattggaggtgccagtggaccccccaccatccagccctgcccaggaggccctggatctcctctttaactgtgagagcaccgaggaggcatctggttcccctgcccgaggccccctcaccgaggctgagcttgccctcttcgaccccttctccaagggtgacccgttgcctgcccgccaggaaggtgtgaagaagaaggcagctgagtacctgaagcgggcagaggagatcctgcgcctgcacctgtctcaactcccaccctaacagggagtgggccattccctgggactctcactcctgcactgccagccccttctcctctccccagggcctggccctacctcctggtcttgtaattacaggagccatttctgtaggtaactggaccaagaatgagaaaaataatgaattcttagctccctgattacacctgccaccttggaatccaggactcacacttctgaccctgcctgtctttttggggtttttttgagttggagtctcgctgtgtcgcccagactggagtgcagtggtgggatcgcggctcactgcaacctccacctcccaggttcaagcagttctcctgtctcagcctccccagtagctgagattgcaggcacatgccaccacgcccagctaatattttgtattttcagtagggacggggttacaccatgttggccaggctggtctcgaactcctgacctcaagtgatccacccgcctcagtctcccaaagtgctgagattacaggcatgagtcactacgcccggcccatgtctgtctgtcttgatgtgtgagcagcagctgtggtcattaaaccattagtttacccctctagaactggggtctgcaaactcccacctgcagccaaatctggcccacctcctttttaatgtaagggctgtgagagtggtttttactttttttaatgattaaaaaaatcaaaataatattctgtgacaatgacaggtgaaatttatatgtgacaagtgaaaattatatgaaatttaagagtccataaataaaatttgttggaacacaaaaaaaaaaaaaaaaaaa

SEQ ID NO: 75 - protein, 255 aa, Homo sapiens
MSRQAKDDFLRHYTVSDPRTHPKGYTEYKVTAQFISKKDPEDVKEVVVWKRYSDFRKLHGDLAYTHRNLFRRLEEFPAFPRAQVFGRFEASVIEERRKGAEDLLRFTVHIPALNNSPQLKEFFRGGEVTRPLEVSRDLHILPPPLIPTPPPDDPRLSQLLPAERRGLEELEVPVDPPPSSPAQEALDLLFNCESTEEASGSPARGPLTEAELALFDPFSKGDPLPARQEGVKKKAAEYLKRAEEILRLHLSQLPP

(19) United States
(12) Patent Application Publication
(10) Pub. No.: US 2008/0219981 A1
(43) Pub. Date: Sep. 11, 2008

(54) Diagnostic Kit for Solid Cancer and Medicament for Solid Cancer Therapy

(76) Inventors: Hideki Shimada, Chiba (JP); Takeshi Tomonaga, Chiba (JP); Takaki Hiwasa, Chiba (JP); Kazuyuki Matsushita, Chiba (JP); Takenori Ochiai, Chiba (JP); Fumio Nomura, Chiba (JP); Masaki Takiguchi, Chiba (JP)

Correspondence Address: BIRCH STEWART KOLASCH & BIRCH, PO BOX 747, FALLS CHURCH, VA, 22040-0747, US

(21) Appl. No.: 10/594,771

(22) PCT Filed: Mar. 24, 2005
(86) PCT No.: PCT/JP2005/006222

§ 371 Date: Sep. 29, 2006

(30) Foreign Application Priority Data
Mar. 29, 2004 (JP) 2004-095732

(51) Int. Cl. A61K 39/395; C07K 14/435; C07H 21/04; G01N 33/574

(52) U.S. Cl. 424/138.1; 530/350; 536/23.5; 435/7.23

(57) ABSTRACT

The present invention provides novel solid cancer antigenic proteins, and diagnostic kits for solid cancer and therapeutic agents for solid cancer based on the antigenic proteins. Specifically, the present invention provides a human solid cancer antigenic polypeptide having an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 48, 50, 52, 54, 56, 59, 61, 63, 65, 67, 69, 71, 73, and 75.

### TECHNICAL FIELD

The present invention relates to diagnostic kits for solid cancer and medicaments for preventing or treating solid cancer.

### BACKGROUND ART

All solid cancers are characterized by the presence of malignant tumors. Examples thereof include esophageal cancer, gastric cancer, lung cancer, kidney cancer, thyroid cancer, parotid gland cancer, head and neck cancer, soft tissue and bone sarcoma, urinary tract cancer, bladder cancer, uterine cancer, liver cancer, breast cancer, ovarian cancer, and fallopian tube cancer. Thus, in particular, treatment of progressive solid cancer is difficult, resulting in death in many cases. Therefore, early tumor detection is the most important objective of therapeutic measures against solid cancer.

Hitherto, tumor markers such as CEA and CA19-9 have been reported and used for diagnosis of the solid cancers listed above and for the prognosis thereof. However, with any tumor marker, the percentage of positive results is as low as 20% to 30%. In particular, during the early stage of cancer, negative results are obtained when using most such markers. In addition, since poor therapeutic outcomes are obtained in the case of the progressive solid cancers listed above, early detection of cancer is the most effective. Thus, novel and effective tumor markers have been expected to be discovered.

In addition, molecular biological diagnostic methods using antigenic protein markers are disclosed in JP Patent Publication No. 7-51065 A (1995), WO00/060073, and JP Patent Publication No. 2000-511536 A, for example. In addition, a SEREX method (serological identification of antigens by recombinant expression cloning) whereby proteins produced using mRNA of tumor cells obtained from a cancer patient are subjected to screening with the patient's autoserum has been reported (Proc. Natl. Acad. Sci. USA 92: 11810-11813, 1995 and U.S. Pat. No. 5,698,396). Further, it has been reported that the aforementioned SEREX method was used to isolate cancer antigens recognized by IgG antibodies in the cases of malignant melanoma, kidney cancer, esophageal cancer, colorectal cancer, and lung cancer, for example (Int. J. Cancer 72: 965-971, 1997; Cancer Res. 58: 1034-1041, 1998; Int. J. Cancer 29: 652-658, 1998; Int. J. Oncol. 14: 703-708, 1999; Cancer Res. 56: 4766-4772, 1996; Hum. Mol. Genet. 6: 33-39, 1997). Further, JP Patent Publication No. 2001-333782 A discloses malignant melanoma antigenic proteins identified by the SEREX method, DNA sequences encoding the proteins, and a method for diagnosing malignant melanoma using such proteins and DNA sequences. However, in order to improve diagnostic accuracy with regard to solid cancer, it is essential to prepare as many protein markers having high antigenicity as possible so as to use them in combination.

Meanwhile, as therapeutic methods for solid cancer, surgical extraction of cancer tissue, systemic administration of anticancer drugs, and the like have been carried out. However, as described above, in the case of progressive solid cancer, such therapeutic methods have had few effects. Even when solid cancer is found during the early stage, these therapeutic methods impose heavy physical burdens on patients, which has been problematic.

### DISCLOSURE OF THE INVENTION

As described above, it has been pointed out that the molecular biological diagnostic method using cancer tissue-specific antigenic protein markers is effective as a method for early diagnosis of solid cancer. Thus, several new antigenic protein markers have been suggested. However, in order to improve diagnostic accuracy with regard to solid cancer, it is essential to prepare as many protein markers having high antigenicity as possible so as to use them in combination.

In addition, since such antigenic protein markers are dominantly expressed in solid cancer tissue, the markers are expected to be applied to therapeutic methods of selectively targeting cancer tissue.

Thus, it is an objective of the present invention to provide novel solid cancer antigenic proteins, diagnostic kits for solid cancer, and therapeutic agents for solid cancer based on the antigenic proteins.

As a result of intensive studies to attain above objectives, the present inventors have found novel antigenic polypeptides that are specific to human solid cancer, and have found that solid cancer can be diagnosed and that solid cancer can be prevented or treated with the use of the expression of such antigenic polypeptides. Thus, this has led to the completion of the present invention.

That is, the present invention relates to a human solid cancer antigenic polypeptide having an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 48, 50, 52, 54, 56, 59, 61, 63, 65, 67, 69, 71, 73, and 75.

The present invention also relates to a polynucleotide encoding the human solid cancer antigenic polypeptide.

Also, the present invention relates to a polynucleotide having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 46, 47, 49, 51, 53, 55, 57, 58, 60, 62, 64, 66, 68, 70, 72, and 74.

The present invention further relates to a diagnostic kit for solid cancer comprising a means of detecting the expression of at least one human solid cancer antigenic polypeptide in a sample derived from a subject, characterized in that the human solid cancer antigenic polypeptide has an amino acid sequence selected from the group consisting of SEQ ID NOS: 2, 4, 6, 8, 1.0, 12, 14, 16, 18, 20, 22, 24, 26, 28, 30, 32, 34, 36, 38, 40, 42, 44, 48, 50, 52, 54, 56, 59, 61, 63, 65, 67, 69, 71, 73, and 75.

The present invention also relates to a diagnostic kit for solid cancer comprising a means of detecting the expression of at least one human solid cancer antigenic polypeptide in a sample derived from a subject, characterized in that the human solid cancer antigenic polypeptide is encoded by a polynucleotide having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 46, 47, 49, 51, 53, 55, 57, 58, 60, 62, 64, 66, 68, 70, 72, and 74.

Examples of the means of detecting the expression of the human solid cancer antigenic polypeptide(s) include the solid cancer antigenic polypeptide or a partial peptide thereof, an antibody against the solid cancer antigenic polypeptide, and a primer or probe comprising polynucleotide consisting of the entire or a partial sequence of a polynucleotide encoding the solid cancer antigenic polypeptide or a complementary sequence thereof.

In addition, the means of detecting the expression of the human solid cancer antigenic polypeptide(s) may be immobilized on a solid phase and/or labeled.

Examples of the solid cancer include colorectal cancer, esophageal cancer, gastric cancer, and breast cancer.

Examples of the sample include serum, blood, hemocytes, and tissue.

The present invention also relates to a medicament for preventing or treating solid cancer comprising a means of inhibiting the functions or expression of at least one human solid cancer antigenic polypeptide, characterized in that the human solid cancer antigenic polypeptide has an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 48, 50, 52, 54, 56, 59, 61, 63, 65, 67, 69, 71, 73, and 75.

The present invention further relates to a medicament for preventing or treating solid cancer comprising a means of inhibiting the functions or expression of at least one human solid cancer antigenic polypeptide, characterized in that the human solid cancer antigenic polypeptide is encoded by a polynucleotide having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 46, 47, 49, 51, 53, 55, 57, 58, 60, 62, 64, 66, 68, 70, 72, and 74.

Examples of the means of inhibiting the functions or expression of the human solid cancer antigenic polypeptide(s) include an antibody against the solid cancer antigenic polypeptide, a means capable of inhibiting transcription of a gene encoding the solid cancer antigenic polypeptide, and a means capable of inhibiting translation of a gene encoding the solid cancer antigenic polypeptide.

Further, the present invention relates to a medicament for preventing or treating solid cancer comprising a gene encoding a prophylactic or therapeutic agent for solid cancer and a means of targeting to human solid cancer.

Examples of the means of targeting to human solid cancer include an antibody against a solid cancer antigenic polypeptide and a nucleotide sequence of an expression control region of a polynucleotide encoding a solid cancer antigenic polypeptide.

### BRIEF DESCRIPTION OF THE DRAWINGS

FIG. 1 A shows results of a comparison in terms of the specific expression of an antigenic protein between cancer tissue and normal tissue of a colorectal cancer patient.

FIG. 1 B shows results of a comparison in terms of the specific expression of an antigenic protein between cancer tissue and normal tissue of a colorectal cancer patient.

FIG. 2 shows results of Western blotting analysis showing reactivity of an antigenic polypeptide expressed by a polynucleotide having the nucleotide sequence set forth in SEQ ID NO: 41 and an antibody in the serum of a patient.

FIG. 3 shows results of Western blotting analysis showing reactivity of an antigenic polypeptide expressed by a polynucleotide having the nucleotide sequence set forth in SEQ ID NO: 43 and an antibody in the serum of a patient.

FIG. 4 shows results of Western blotting analysis showing reactivity of an antigenic polypeptide expressed by a polynucleotide having the nucleotide sequence set forth in SEQ ID NO: 45 and an antibody in the serum of a patient.

FIG. 5 shows results of Western blotting analysis showing reactivity of an antigenic polypeptide expressed by a polynucleotide having the nucleotide sequence set forth in SEQ ID NO: 46 and an antibody in the serum of a patient.

FIG. 6 shows results of Western blotting analysis showing reactivity of an antigenic polypeptide expressed by a polynucleotide having the nucleotide sequence set forth in SEQ ID NO: 47 and an antibody in the serum of a patient.

FIG. 7 shows results of Western blotting analysis showing reactivity of an antigenic polypeptide expressed by a polynucleotide having the nucleotide sequence set forth in SEQ ID NO: 49 and an antibody in the serum of a patient.

FIG. 8 shows results of Western blotting analysis showing reactivity of an antigenic polypeptide expressed by a polynucleotide having the nucleotide sequence set forth in SEQ ID NO: 51 and an antibody in the serum of a patient.

FIG. 9 shows results of Western blotting analysis showing reactivity of an antigenic polypeptide expressed by a polynucleotide having the nucleotide sequence set forth in SEQ ID NO: 53 and an antibody in the serum of a patient.

FIG. 10 shows results of Western blotting analysis showing reactivity of an antigenic polypeptide expressed by a polynucleotide having the nucleotide sequence set forth in SEQ ID NO: 55 and an antibody in the serum of a patient.

FIG. 11 shows results of Western blotting analysis showing reactivity of an antigenic polypeptide expressed by a polynucleotide having the nucleotide sequence set forth in SEQ ID NO: 57 and an antibody in the serum of a patient.

FIG. 12 shows results of Western blotting analysis showing reactivity of an antigenic polypeptide expressed by a polynucleotide having the nucleotide sequence set forth in SEQ ID NO: 58 and an antibody in the serum of a patient.

FIG. 13 shows results of Western blotting analysis showing reactivity of an antigenic polypeptide expressed by a polynucleotide having the nucleotide sequence set forth in SEQ ID NO: 60 and an antibody in the serum of a patient.

FIG. 14 shows results of Western blotting analysis showing reactivity of an antigenic polypeptide expressed by a polynucleotide having the nucleotide sequence set forth in SEQ ID NO: 62 and an antibody in the serum of a patient.

FIG. 15 shows results of Western blotting analysis showing reactivity of an antigenic polypeptide expressed by a polynucleotide having the nucleotide sequence set forth in SEQ ID NO: 64 and an antibody in the serum of a patient.

FIG. 16 shows results of Western blotting analysis showing reactivity of an antigenic polypeptide expressed by a polynucleotide having the nucleotide sequence set forth in SEQ ID NO: 66 and an antibody in the serum of a patient.

FIG. 17 shows results of Western blotting analysis showing reactivity of an antigenic polypeptide expressed by a polynucleotide having the nucleotide sequence set forth in SEQ ID NO: 68 and an antibody in the serum of a patient.

FIG. 18 shows results of Western blotting analysis showing reactivity of an antigenic polypeptide expressed by a polynucleotide having the nucleotide sequence set forth in SEQ ID NO: 70 and an antibody in the serum of a patient.

FIG. 19 shows results of Western blotting analysis showing reactivity of an antigenic polypeptide expressed by a polynucleotide having the nucleotide sequence set forth in SEQ ID NO: 72 and an antibody in the serum of a patient.

FIG. 20 shows results of Western blotting analysis showing reactivity of an antigenic polypeptide expressed by a polynucleotide having the nucleotide sequence set forth in SEQ ID NO: 74 and an antibody in the serum of a patient.

### BEST MODE FOR CARRYING OUT THE INVENTION

Hereafter, the present invention will be described in detail. The present application claims a priority from Japanese Patent Application No. 2004-95732 filed on Mar. 29, 2004, and the entire contents disclosed in the specification and/or drawings thereof are hereby incorporated.

### 1. Novel Human Solid Cancer Antigenic Polypeptides

The present invention is based on novel antigenic polypeptides that are specific to human solid cancer. With the consent of colorectal cancer patients, the present inventors extracted proteins from normal tissue and cancer tissue of surgical samples taken from the patients, followed by analysis via two-dimensional electrophoresis (e.g., Electrophoresis 22: 3019-3025, 2001). Thus, we have found 20 types of antigenic polypeptides (set forth in numbers 1 to 20 in table 1) that are specifically expressed in solid cancer cells and that have not been known to have the function as tumor markers (see Example 1). In addition, serum obtained from esophageal cancer, gastric cancer, colorectal cancer, and breast cancer patients with their consent was analyzed by a SEREX method (Proc. Natl. Acad. Sci. USA 92: 11810-11813, 1995; U.S. Pat. No. 5,698,396). Accordingly, we have also found antigenic polypeptides (set forth in numbers 21 to 39 in table 1) against 19 types of specific antibodies that exist exclusively in serum of esophageal cancer, gastric cancer, colorectal cancer, and breast cancer patients (see Example 2). These solid cancer antigenic polypeptides are shown in table 1.

TABLE 1

| | | Registered | Nucleotide | Amino acid | |
|---|---|---|---|---|---|
| No. | Name | number | sequence | sequence | Clone name |
| 1 | Malate dehydrogenase 2 | NP_005909, | 1 | 2 | |
| | | NM_005918 | | | |
| 2 | Tropomyosin 4 | NP_003281, | 3 | 4 | |
| | | NM_003290 | | | |
| 3 | FK506 binding protein 4 | NP_002005, | 5 | 6 | |
| | | NM_002014 | | | |
| 4 | Chaperonin-containing TCP1, subunit 6A | NP_001753, | 7 | 8 | |
| | | NM_001762 | | | |
| 5 | Serine protease inhibitor, clade H, | NP_001226, | 9 | 10 | |
| | collagen binding protein 1 | NM_001235 | | | |
| 6 | Sulfide dehydrogenase-like | NP_067022, | 11 | 12 | |
| | | NM_021199 | | | |
| 7 | Hydroxysteroid(17-β)dehydrogenase 4 | NP_000405, | 13 | 14 | |
| | | NM_000414 | | | |
| 8 | Stress-induced-phosphoprotein 1 | NP_006810, | 15 | 16 | |
| | | NM_006819 | | | |
| 9 | Heterogeneous nuclear ribonucleoprotein L | NP_001524, | 17 | 18 | |
| | | NM_001533 | | | |
| 10 | Heterogeneous nuclear ribonucleoprotein U | NP_004492, | 19 | 20 | |
| | | NM_004501 | | | |
| 11 | Matrine 3 | NP_061322, | 21 | 22 | |
| | | NM_018834 | | | |
| 12 | Annexin A3 | NP_005130, | 23 | 24 | |
| | | NM_005139 | | | |
| 13 | PTK9L protein tyrosine kinase 9-like | NP_009215, | 25 | 26 | |
| | | NM_007284 | | | |
| 14 | splicing factor, arginine/serine-rich 1 | NP_008855, | 27 | 28 | |
| | | NM_006924 | | | |
| 15 | Thiosulfate sulphurtransferase | NP_003303, | 29 | 30 | |
| | | NM_003312 | | | |
| 16 | S-adenosylhomocysteine hydrolase | NP_000678, | 31 | 32 | |
| | | NM_000687 | | | |
| 17 | GDP-mannose 4,6-dehydratase | NP_001491, | 33 | 34 | |
| | | NM_001500 | | | |
| 18 | Hydroxyacyl dehydrogenase, | NP_000173, | 35 | 36 | |
| | subunit A | NM_000182 | | | |
| 19 | Prolyl-4-hydroxylase β subunit | NP_000909, | 37 | 38 | |
| | | NM_000918 | | | |
| 20 | Peroxyredoxin 5 | NP_036226, | 39 | 40 | |
| | | NM_012094 | | | |
| 21 | Progesterone receptor membrane component 2 | NM_006320 | 41 | 42 | K35-1-1 |
| 22 | MAP kinase interacting serine/threonine | NM_199054 | 43 | 44 | K30-1-1 |
| | kinase 2 | | | | |
| 23 | EST: 601191782F1 | BE264462 | 45 | | 12N3-1 |
| 24 | EST: 602301679F1 | BG032310 | 46 | | 12O1-1 |
| 25 | Additional sex combs-like 1 | NM_015338 | 47 | 48 | 14A1-1-1 |
| 26 | Forkhead box A1 | NM_004496 | 49 | 50 | 18G3-1 |
| 27 | Retinoic acid induced 16 | NM_022749 | 51 | 52 | 19C1-1 |
| 28 | RIKEN cDNA 5730528L13-like gene | NM_080655 | 53 | 54 | 19F1-1 |
| 29 | Lysine tRNA synthase | BC004132 | 55 | 56 | 19F1-2 |
| 30 | EST: AGENCOURT_15657942 | CF597227 | 57 | | 6BD3-1 |
| 31 | KDEL endoplasmic reticulum protein | NM_006801 | 58 | 59 | 14H1-2-1 |
| | retention receptor 1 | | | | |
| 32 | Lysosomal associated protein transmembrane | NM_018407 | 60 | 61 | 18B2-1 |
| | 4 beta | | | | |
| 33 | Protein phosphatase 1, | NM_002708 | 62 | 63 | 18G1-1 |
| | catalytic subunit, α isoform | | | | |
| 34 | Peroxyredoxin 3 | NM_006793 | 64 | 65 | 20J4-1 |
| 35 | Aldo-keto reductase family 1, member C3 | NM_003739 | 66 | 67 | 19M2 |
| 36 | Ubiquitin-conjugating enzyme E2I | BC000744 | 68 | 69 | 10Q3-1 |
| 37 | Phosphatidic acid phosphatase, Type 2C | NM_003712 | 70 | 71 | 14A1-1-2 |
| 38 | Beta-catenin interacting protein 1 | NM_020248 | 72 | 73 | 14B1-2-1 |
| 39 | Sorting nexin 15 | NM_147777 | 74 | 75 | 14H2-1-1 |

Note that, in accordance with the present invention, the term “polypeptide(s)” means molecules such as proteins and peptides, which are composed of a plurality of amino acid residues that are bound to one another via an amide bond (peptide bond). Also, the term “polynucleotide” means a molecule in which a plurality of phosphate esters of nucleosides each having a purine or pyrimidine that is bind to a sugar via a β-N-glycoside bond (ATPs, GTPs, CTPs, or UTPs; or dATPs, dGTPs, dCTPs, or dTTPs) are bound to each other.

In addition, the present invention encompasses nucleotide sequences derived from the nucleotide sequences listed in table 1 by addition, deletion, or substitution of one to several nucleotides. In addition, based on such nucleotide variation, the present invention also encompasses amino acid sequences derived from the amino acid sequences listed in table 1 by addition, deletion, or substitution of one to several amino acid residues.

Further, the term “serum antibody” means an IgG antibody that exists in the serum of a solid cancer patient and that binds to a solid cancer antigenic polypeptide. Moreover, the term “antibody” means a polyclonal or monoclonal antibody prepared by using a solid cancer antigenic polypeptide or a partial fragment thereof as an immunogen.

Other terms and concepts used in the present invention will be defined in more detail in the following embodiments and Examples. In addition, based on known literature and the like, persons skilled in the art would be readily and reliably able to use various techniques so as to carry out the present invention, except for techniques that are clearly described herein with references. For instance, preparation of pharmaceuticals for the preparation of the medicaments of the present invention is described in Remington's Pharmaceutical Sciences, 18th Edition, ed. A. Gennaro, Mack Publishing Co., Easton, Pa., 1990. Also, genetic engineering techniques and molecular biological techniques used in the present invention are described in, for example, Sambrook and Maniatis, Molecular Cloning—A Laboratory Manual, Cold Spring Harbor Laboratory Press, New York, 1989 and Ausubel, F. M. et al., Current Protocols in Molecular Biology, John Wiley & Sons, New York, N.Y., 1995.

The term “solid cancer antigenic polypeptide” refers to a polypeptide expressed by any of the 39 genes or ESTs (Expressed Sequence Tags) shown in table 1. These gene products are known to have various functions; however, their specific expression in solid cancer has not been known. Herein, in accordance with the present invention, an expression product of an EST is defined as a “polypeptide encoded by a polynucleotide comprising the nucleotide sequence set forth in SEQ ID NO: 45, 46, or 57.”

Moreover, as described in Examples below, genes and peptides set forth in SEQ ID NOS: 1 to 40 are identified via two-dimensional electrophoresis, and genes, ESTs, and peptides set forth in SEQ ID NOS: 41 to 75 are identified via the SEREX method.

### 2. Diagnostic Kits for Solid Cancer

As described above, solid cancer antigenic polypeptides shown in table 1 are specifically expressed in human solid cancer. Thus, it becomes possible to diagnose a subject as having human solid cancer by detecting the expression of such a solid cancer antigenic polypeptide in a sample derived from the subject.

A diagnostic kit for solid cancer of the present invention (hereafter sometimes referred to as “the present diagnostic kit for solid cancer”) includes a means of detecting the expression of at least one human solid cancer antigenic polypeptide in a sample derived from a subject.

The present diagnostic kit for solid cancer is a reagent kit used to make a diagnosis of solid cancer. A variety of such kits are commercially available in accordance with types of test components. Also, the present diagnostic kit for solid cancer may comprise components used in publicly known kits, except that a means of detecting the expression of a human solid cancer antigenic polypeptide is used (e.g., solid cancer antigenic polypeptides, antibodies, primers, and probes).

Further, with the use of the present diagnostic kit for solid cancer, it is possible to diagnose a subject as having solid cancer. Examples of solid cancer include, but are not limited to, colorectal cancer, esophageal cancer, gastric cancer, lung cancer, kidney cancer, thyroid cancer, parotid gland cancer, head and neck cancer, soft tissue and bone sarcoma, urinary tract cancer, bladder cancer, uterine cancer, liver cancer, breast cancer, ovarian cancer, and fallopian tube cancer. Preferably, the kit can be used for diagnosis of colorectal cancer, esophageal cancer, gastric cancer, or breast cancer.

Herein, examples of a means of detecting the expression of a solid cancer antigenic polypeptide, the protein expression thereof, the antibody expression thereof, and the gene expression thereof include:

(1) an antibody against the solid cancer antigenic polypeptide;

(2) a solid cancer antigenic polypeptide; and

(3) a probe or primer designed based on a polynucleotide encoding the solid cancer antigenic polypeptide.

Hereafter, these means will be described in detail.

### (1) Antibody Against a Solid Cancer Antigenic Polypeptide

An antibody against a solid cancer antigenic polypeptide is able to bind to a solid cancer antigenic polypeptide expressed in cancer. Thus, by detecting the reaction between the antibody and a solid cancer antigenic polypeptide in a sample, it is possible to diagnose whether or not the sample is derived from a cancer patient or a subject at high risk of developing solid cancer.

An antibody against a solid cancer antigenic polypeptide can be a polyclonal or monoclonal antibody. Examples thereof include an entire molecule and Fab, F(ab′)₂, and Fv fragments that can bind to the epitope of a solid cancer antigenic polypeptide. For instance, in the case of a polyclonal antibody, such antibody can be obtained by immunizing an animal with an immunogen such as an antigenic polypeptide or a partial fragment thereof and recovering from the serum of the animal. Alternatively, such antibody can be prepared by administering the expression vector for eukaryotic cells described above into the muscle or skin of an animal via injection or a gene gun, and collecting the serum. Examples of such animal include mice, rats, rabbits, goats, and chickens.

Further, the monoclonal antibody can be produced in accordance with a known method for producing monoclonal antibodies (“Monoclonal Antibody (Tan-Clone Kotai),” cowritten by Hideaki Nagamune and Hiroshi Terada, Hirokawa Shoten, 1990; “Monoclonal Antibody” James W. Goding, third edition, Academic Press, 1996).

Furthermore, an antibody against a solid cancer antigenic polypeptide can be an antibody labeled with a labeling substance. The details of such labeled antibody are described above.

When the expression of a solid cancer antigenic polypeptide in a sample derived from a subject is detected using an antibody against a solid cancer antigenic polypeptide and diagnosis of human solid cancer is carried out, the sample derived from the subject is examined for the presence or absence of an antibody against a solid cancer antigenic polypeptide or an antigenic polypeptide that binds to a labeled antibody thereof. If the antigenic polypeptide is detected in the sample, the subject is determined to be a solid cancer patient or a subject at high risk of developing solid cancer. That is, the antibody or labeled antibody used herein specifically binds to an antigenic polypeptide expressed in a solid cancer cell. Thus, a sample comprising an antigenic polypeptide that is bound to an antibody can be determined as a sample of a solid cancer patient or a subject at high risk of developing solid cancer. In such case, preferably 2 or more, more preferably 5 or more, even more preferably 10 or more, most preferably 15 to 39 antibodies are examined in terms of binding with antigenic polypeptides in a sample. Such sample to be used is not particularly limited as long as a solid cancer antigenic polypeptide is expressed therein. Examples thereof include blood, hemocytes (e.g., mononuclear cells), and tissue.

In another embodiment, a method wherein an antibody and an antigenic polypeptide are allowed to bind to each other in a liquid phase system is used. For instance, a labeled antibody is allowed to come into contact with a sample such that the labeled antibody and an antigenic polypeptide bind to each other. Then, such conjugate is separated such that a signal from the label is detected in a manner similar to that described above.

In another method of diagnosis in a liquid phase system, an antibody (primary antibody) against a solid cancer antigenic polypeptide is allowed to come into contact with a sample such that a primary antibody and the antigenic polypeptide bind to each other. Then, a labeled antibody (secondary antibody) is allowed to bind to the thus obtained conjugate such that the signal from the label of the conjugate comprising the three components is detected. Alternatively, in order to intensify the signal, an unlabeled secondary antibody is allowed to bind to a conjugate of an antibody and an antigenic polypeptide such that a labeling substance may be allowed to bind to the secondary antibody. For instance, such binding of a labeling substance to a secondary antibody can be carried out in a manner such that a secondary antibody and a labeling substance are preliminarily biotinylated and avidinylated, respectively. Also, an antibody (tertiary antibody) that recognizes a partial region of a secondary antibody (e.g., Fc region) is used for labeling so that the tertiary antibody is allowed to bind to the secondary antibody. In addition, as a primary antibody and a secondary antibody, a monoclonal antibody can be used. Alternatively, either the primary antibody or the secondary antibody may be a polyclonal antibody. Separation of such conjugate from a liquid phase and signal detection can be carried out in a manner similar to that described above.

In another embodiment, a method for examining binding between an antibody and an antigenic polypeptide in a solid phase system is used. The method involving a solid phase system is preferable in terms of detection of minute amounts of antigenic polypeptides and facilitation of operations. That is, in accordance with the method involving a solid phase system, antibodies (primary antibodies) against solid cancer antigenic polypeptides are immobilized on a solid phase (e.g., a resin plate, a membrane, or beads), antigenic polypeptides are allowed to bind to the thus immobilized antibodies, unbound peptides are removed by washing, labeled antibodies (secondary antibodies) are allowed to bind to conjugates of the antibody and the antigenic polypeptide remaining on the plate, for example, and signals of the secondary antibodies are detected. This method is a so-called “sandwich method.” Such method is widely used as “ELISA (enzyme linked immunosorbent assay)” using an enzyme as a marker. In the method, the both primary antibody and the secondary antibody may be monoclonal antibodies. Alternatively, either the primary antibody or the secondary antibody may be a polyclonal antibody. Signal detection can be carried out in a manner similar to that described above.

### (2) Solid Cancer Antigenic Polypeptide

Since solid cancer antigenic polypeptides are expressed by cancer cells, the serum of a patient having cancer contains an antibody (serum antibody) against an expressed solid cancer antigenic polypeptide. Thus, by examining the reaction between a solid cancer antigenic polypeptide and a serum antibody, the expression of a solid cancer antigenic polypeptide in a subject can be detected. Examples of such solid cancer antigenic polypeptide that can be used include the antigenic polypeptides shown in table 1 and partial peptides thereof. Herein, the term “antigenic polypeptide” means the antigenic polypeptides shown in table 1, and also partial peptides of the antigenic polypeptides that comprise at least 6 amino acids, preferably 6 to 500 amino acids, and more preferably 8 to 50 amino acids.

These antigenic polypeptides can be prepared by preparing RNA via in vitro transcription from a recombinant expression vector comprising a polynucleotide having a nucleotide sequence listed in table 1, for example, carrying out in vitro translation using the RNA as a template, and expressing an antigenic peptide in vitro. Alternatively, when a recombinant expression vector is introduced into a prokaryotic cell such as E coli. or Bacillus subtilis, or a eukaryotic cell such as yeast, an insect cell, or a mammalian cell such that a transformed cell is produced, it is possible to allow such transformed cell to express an antigenic polypeptide.

When an antigenic polypeptide is expressed via in vitro translation, a polynucleotide encoding an antigenic polypeptide is inserted into a vector comprising an RNA polymerase promoter to construct a recombinant expression vector. Then, the obtained vector is added to an in vitro translation system such as a rabbit reticulocyte lysate or a wheat germ extract that contains an RNA polymerase corresponding to the promoter. Thus, an antigenic polypeptide can be produced in vitro. Examples of an RNA polymerase promoter include T7, T3, and SP6. Examples of a vector comprising such RNA polymerase promoter include pKA1, pCDM8, pT3/T7 18, pT7/3 19, and pBluescript II.

When an antigenic polypeptide is allowed to be expressed in an organism such as E coli., an expression vector is produced by ligating a polynucleotide to a vector having a replication origin that is replicable in a microorganism, a promoter, a ribosome binding site, a DNA cloning site, and a terminator. A host cell is then transformed with the expression vector, and the thus obtained transformant is subjected to culture. Thus, an antigenic polypeptide encoded by the polynucleotide can be expressed using microorganisms. In such case, it is also possible to express an antigenic polypeptide as a fusion protein with another protein. Examples of an E coli. expression vector include pUC, pBluescript II, a pET expression system, and a pGEX expression system.

When an antigenic polypeptide is allowed to be expressed in a eukaryotic cell, a recombinant vector is produced by inserting a polynucleotide encoding an antigenic polypeptide into a eukaryotic cell expression vector having a promoter, a splicing region, a poly(A) addition site, and the like. Then, the recombinant vector is introduced into a eukaryotic cell. Accordingly, the antigenic polypeptide can be expressed in the transformed eukaryotic cell. Examples of such expression vector include pKA1, pCDM8, pSVK3, pMSG, pSVL, pBK-CMV, pBK-RSV, EBV vector, pRS, pcDNA3, pMSG; and pYES2. In addition, when pIND/V5-His, pFLAG-CMV-2, pEGFP-N1, pEGFP-C1, and the like are used as expression vectors, it is also possible to express an antigenic polypeptide as a fusion protein to which various types of tags such as a His tag, a FLAG tag, a myc tag, an HA tag, and GFP have been added. Examples of such eukaryotic cell that is generally used include cultured mammalian cells such as monkey kidney cells COS 7 and Chinese hamster ovary (CHO) cells, budding yeast, fission yeast, silkworm cells, and Xenopus oocytes. In addition, any eukaryotic cell may be used as long as it can express an antigenic polypeptide. When an expression vector is introduced into a eukaryotic cell, known methods such as an electroporation method, a calcium phosphate method, a liposome method, and a DEAE-dextran method may be used.

When isolation and purification of an antigenic polypeptide of interest from a culture product is carried out following expression of the antigenic polypeptide using prokaryotic cells or eukaryotic cells, known separation techniques can be used in combination. Examples thereof include treatment using a denaturing agent such as urea or a surfactant, ultrasonication, enzymatic digestion, salting-out or solvent precipitation, dialysis, centrifugation, ultrafiltration, gel filtration, SDS-PAGE, isoelectric focusing, ion exchange chromatography, hydrophobic chromatography, affinity chromatography, and reversed phase chromatography.

In addition, recombinant antigenic polypeptides obtained via the methods described above include a fusion protein of such polypeptide and any other protein. For instance, such fusion protein comprises glutathione S-transferase (GST) or green-fluorescent protein (GFP). Further, peptides expressed in transformed cells are sometimes subjected to various forms of post-translational modification in the cells. Thus, modified peptides can be used as antigenic polypeptides. Examples of such post-translational modification include removal of N-terminal methionine, N-terminal acetylation, glycosylation, partial hydrolysis due to intracellular protease, myristoylation, isoprenylation, and phosphorylation.

In order to detect the expression of a solid cancer antigenic polypeptide in a sample derived from a subject using solid cancer antigenic polypeptides, the presence or absence of at least one serum antibody in a sample of a subject that binds to a solid cancer antigenic polypeptide is examined. If the serum antibody is found in the serum of the subject, the subject is determined to be a solid cancer patient or a subject at high risk of developing solid cancer. Specifically, solid cancer antigenic polypeptides bind to serum antibodies (IgG) derived from a solid cancer patient. Thus, when such polypeptides are allowed to react with the serum of a subject, the serum sample is found to contain serum antibodies that are bound to the polypeptides, the sample can be determined to be derived from a solid cancer patient or a subject at high risk of developing solid cancer. In such case, 2 or more, preferably 5 or more, more preferably 10 or more, and most preferably 15 to 39 antigenic polypeptides are examined in terms of binding with antibodies. Further, other known solid cancer markers (e.g., CEA, Cyfra, and SCC-Ag) can be used in combination. Moreover, the sample may be a serum antibody-containing sample, which in turn indicates serum.

Specifically, diagnosis using the present diagnostic kit for solid cancer is carried out by allowing serum of a subject to come into contact with, for example, solid cancer antigenic polypeptides in the diagnostic kit for solid cancer so as to allow the solid cancer antigenic polypeptides to react with IgG antibodies in the serum of the subject in a liquid phase. Further, labeled IgG antibodies that specifically bind to serum IgG antibodies are subjected to reaction such that signals of the labeled IgG antibodies can be detected. Examples of labels used in labeled antibodies include enzymes, radioisotopes, and fluorescent dyes. Such enzymes are not particularly limited as long as they satisfy conditions such as a large turnover number, stability even while being bound to antibodies, and capacity to cause color development specifically to a substrate. Examples of the enzymes that can be used include enzymes used in usual enzyme immunoassay (EIA) such as peroxidase, β-galactosidase, alkaline phosphatase, glucose oxidase, acetylcholine esterase, glucose-6-phosphodehydrogenase, and malate dehydrogenase. Also, enzyme inhibitors, coenzymes, and the like can be used. Binding between these enzymes and antibodies can be carried out by known methods using crosslinkers such as maleimide compounds. As the substrate, known substances can be used depending on the type of enzyme used. For instance, when peroxidase is used as the enzyme, 3,3′,5,5′-tetramethylbenzene can be used, and when alkaline phosphatase is used as the enzyme, paranitrophenol or the like can be used.

When such enzyme is used, enzyme activity is determined by adding a substrate that causes color development as a result of degradation via enzymatic action so as to optically measure the amount of the substrate degraded. Then, the obtained enzyme activity is converted into an amount of binding antibody such that the amount of antibody is calculated based on comparison with a standard.

Examples of radioisotopes that can be used include ¹²⁵I and ³H, which are used in general radioimmunoassay (RIA). When such a radioisotope is used, the radiation dose of the radioisotope is measured using a scintillation counter or the like.

Examples of fluorescent dyes that can be used include fluorescein isothiocyanate (FITC) and tetramethylrhodamine isothiocyanate (TRITC), which are used in general fluorescent antibody methods. When such a fluorescent dye is used, the fluorescence level may be measured using a measuring apparatus equipped with a fluorescence microscope.

Further, examples of labeled antibodies include antibodies to which metals such as manganese and iron are bound. By carrying out in vivo administration of such antibodies to which metals are bound so as to measure the metals via MRI or the like, the presence of serum antibodies, or the expression of solid cancer antigenic polypeptides, can be detected.

Signal detection can be carried out by employing Western blotting analysis, for example. Alternatively, a conjugate of an antigenic polypeptide, a serum antibody, and a labeled IgG antibody is separated by a known separation means (e.g., chromatography, a salting out, an alcohol precipitation, an enzymatic method, or a solid phase method) such that signals of the labeled IgG antibody may be detected.

In addition, at least one antigenic polypeptide is immobilized on a solid phase (e.g., plates, membranes, and beads) such that binding between serum antibodies of a subject and the antigenic polypeptides can be examined on the solid phase. By immobilizing antigenic polypeptides on a solid phase, it becomes possible to readily remove unbound labeled binding molecules. In particular, in accordance with a protein array method wherein a membrane on which several tens of different types of antigenic polypeptides are immobilized is used, different types of a plurality of antibodies can be analyzed in terms of expression in a short time using about 0.01 ml of the serum of a subject.

### (3) Primers or Probes

The present diagnostic kit for solid cancer may comprise a primer or probe that comprises polynucleotide consisting of the entire or a partial sequence of a polynucleotide encoding a solid cancer antigenic polypeptide shown in table 1 or a complementary sequence thereof. The primer or probe specifically binds to mRNA of the antigenic polypeptide expressed in the sample of a subject or cDNA synthesized from the mRNA. Thus, it is possible to detect the expression of a gene encoding an antigenic polypeptide in a sample, which in turn indicates the expression of the antigenic polypeptide.

Such primer and probe can be designed in accordance with techniques known by persons skilled in the art based on nucleotide sequences of polynucleotides encoding antigenic polypeptides, which are set forth in SEQ ID NOS:1, 3, 5, 7, 9, 11, 13, 15, 17, 19, 21, 23, 25, 27, 29, 31, 33, 35, 37, 39, 41, 43, 45, 46, 47, 49, 51, 53, 55, 57, 58, 60, 62, 64, 66, 68, 70, 72, and 74. The following remarks relate to the designing of such primer and probe.

The length of the primer that substantially have its function is preferably 10 or more nucleotides, more preferably 16 to 50 nucleotides, and even more preferably 20 to 30 nucleotides. In addition, the length of the probe that substantially have its function is preferably 10 or more nucleotides, more preferably 16 to 50 nucleotides, and even more preferably 20 to 30 nucleotides.

Upon design, it is preferable to confirm the melting temperature of a primer or probe (Tm). The “Tm” means the temperature at which 50% of an arbitrary polynucleotide chain forms a hybrid with the complementary strand thereof. When template DNA or RNA and a primer or probe anneal or hybridize by forming a duplex, the temperature for annealing or hybridization must be optimized. Meanwhile, when the temperature is excessively decreased, nonspecific reaction takes place. Thus, the temperature is preferably as high as possible. Therefore, the Tm of the primer or probe to be designed is an important factor when carrying out amplification reaction or hybridization. The Tm can be confirmed with the use of known software for designing a primer or probe. Examples of such software that can be used in the present invention include Oligo™ (National Bioscience Inc., (U.S.A.)) and GENETYX (Software Development Co., Ltd. (Japan)). In addition, the Tm can be confirmed by manual calculation without using such software. In such case, calculating formulae based on the nearest neighbor base pair model (nearest neighbor method), the Wallace method, the GC % method, and the like can be used. In accordance with the present invention, the average Tm is preferably about 45° C. to 55° C.

Another example of conditions under which a primer or probe can be specifically anneals or hybridizes involves GC content or the like. Such condition has been known by persons skilled in the art.

The primer and the probe designed as described above can be prepared in accordance with methods known by persons skilled in the art. Further, as known by persons skilled in the art, such primer or probe may contain a sequence such as an additional sequence known as a tag sequence in addition to the sequence for annealing or hybridization. Also, the aforementioned primer or probe to which such additional sequence has been added is within the scope of the present invention.

When expression of solid cancer antigenic polypeptides in a sample derived from a subject is detected, the aforementioned primer and/or probe are used in an amplification reaction or a hybridization reaction, such that the amplification product or hybridization product thereof is detected.

Examples of such sample of interest include feces, blood, and hemocytes (e.g., mononuclear cells). In addition, when carrying out an amplification reaction or a hybridization reaction, a nucleic acid to be tested is prepared from a sample derived from a subject, in general. The test nucleic acid may be DNA or RNA, as long as it is nucleic acid. DNA or RNA can be extracted using methods known in the art according to need. For instance, upon DNA extraction, a method of carrying out phenol extraction and ethanol precipitation and a method using glass beads can be used. Also, upon RNA extraction, a guanidine-cesium chloride ultracentrifugation method, a hot phenol method, an acid guanidinium thiocyanate-phenol-chloroform (AGPC) method, or the like can be used. With the use of a sample or test nucleic acid prepared as described above, an amplification reaction and/or a hybridization reaction described below is carried out.

Expression of a solid cancer antigenic polypeptide in a sample can be detected by carrying out amplification with the use of a primer and test nucleic acid as a template and detecting the specific amplification reaction.

Amplification methods are not particularly limited. However, examples thereof include known methods utilizing principles of polymerase chain reaction (PCR) methods such as PCR, LAMP (loop-mediated isothermal amplification), ICAN (isothermal and chimeric primer-initiated amplification of nucleic acids), RCA (rolling circle amplification), LCR (ligase chain reaction), and SDA (strand displacement amplification). Amplification is carried out until an amplification product can be detected.

For instance, upon PCR, a nucleotide sequence between a pair of primers is synthesized based on a template, DNA to be tested, using DNA polymerase. Upon PCR, amplified fragments can be exponentially amplified with the repetition of a cycle consisting of denaturation, annealing, and synthesis. Optimal conditions for PCR can readily be determined by persons skilled in the art.

In addition, upon RT-PCR, cDNA is produced via reverse transcriptase reaction using RNA to be tested as a template, followed by PCR using a pair of primers and the thus prepared cDNA that serves as a template.

In addition, with the use of amplification techniques such as quantitative PCR including competitive PCR and real-time PCR, quantitative detection can be achieved.

In order to detect whether or not the aforementioned amplification reaction is a specific amplification reaction, known methods whereby amplification products obtained via an amplification reaction can be specifically recognized can be used. For instance, a specific amplification reaction can be detected using agarose gel electrophoresis whereby amplification of amplified fragments in given sizes is confirmed.

Alternatively, labels such as radioisotopes, fluorescent substances, or luminescent substances are allowed to act on dNTP that is incorporated during an amplification reaction such that such label can be detected. Examples of radioisotopes that can be used include ³²P, ¹²⁵I, and ³⁵S. Examples of fluorescent substances that can be used include fluorescein (FITC), sulforhodamine (SR), and tetramethylrhodamine (TRITC). In addition, examples of luminescent substances that can be used include luciferin.

For instance, the label type and the method for introducing a label are not particularly limited. Thus, conventionally known methods can be used. Examples of the method for introducing a label include a random prime method using a radioisotope.

As a method for observing an amplification product into which labeled dNTP has been incorporated, any methods for detecting aforementioned labels known in the art can be used. For instance, when a radioisotope is used as a label, the radioactivity can be measured using a liquid scintillation counter, a γ-counter, or the like. When fluorescence is used as a label, the fluorescence can be detected using a fluorescence microscope, a fluorescent plate reader, or the like.

When a specific amplification reaction is detected as described above, a gene encoding a solid cancer antigenic polypeptide is expressed in a sample, which in turn indicates the expression of a solid cancer antigenic polypeptide. Thus, a subject whose sample shows antigenic polypeptide expression is determined to be a solid cancer patient or subject at high risk of developing solid cancer.

In addition, a sample or test nucleic acid is subjected to a hybridization reaction using a probe, followed by detection of the specific binding (hybrid). Thus, the expression of solid cancer antigenic polypeptides can be detected.

The hybridization reaction must be carried out under stringent conditions that allow a probe to specifically bind to a polynucleotide derived from a solid cancer antigenic polypeptide. Such stringent conditions have been known in the art and thus are not particularly limited. The stringent conditions include a sodium concentration of 10 to 300 mM and preferably 20 to 100 mM and a temperature of 25° C. to 70° C. and preferably 42° C. to 55° C.

When carrying out hybridization, it is possible to add appropriate labels such as fluorescence labels (e.g., fluorescein and rhodamine), radioactive labels (e.g., ³²P), enzyme labels (e.g., alkaline phosphatase and horseradish peroxidase), and biotin labels to a probe. Thus, the present diagnostic kit for solid cancer comprises probes to which labels as descried above have been attached.

Detection using labeled probes includes allowing a sample or test nucleic acid that has been prepared from the sample to come into contact with a probe so as to achieve hybridization. The expression “so as to achieve hybridization” indicates that detection is carried out in an environment (temperature and salt concentration) in which specific binding occurs under the stringent conditions described above. Specifically, a sample or test nucleic acid is immobilized on an adequate solid phase such as a slide glass, a membrane, or a microtiter. Then, labeled probes are added thereto. Thus, a sample or test nucleic acid is allowed to come into contact with the probes such that hybridization reaction is carried out, followed by removal of the nonhybridized probes. Accordingly, the label of the probe that has hybridized with the sample or test nucleic acid is detected. Detection of the label indicates that solid cancer antigenic polypeptides are expressed in the sample. Therefore, a subject whose sample shows expression of an antigenic polypeptide is diagnosed as being a solid cancer patient or a subject at high risk of developing solid cancer.

In addition, when the concentration of the label is determined to be an index, it is also possible to carry out quantitative detection. Examples of a detection method using labeled probes include Southern hybridization, Northern hybridization, and FISH (fluorescent in situ hybridization).

Further, when the present diagnostic kit for solid cancer is used to make a diagnosis, the expression level of solid cancer antigenic polypeptides in a sample derived from a subject is measured. If the expression level of at least one solid cancer antigenic polypeptide is higher than that of a healthy individual, the subject is determined to be a solid cancer patient or subject at high risk of developing solid cancer. In terms of specific criteria for the expression level of a solid cancer antigenic polypeptide, the expression level of a subject is 10% or more, preferably 30% or more, more preferably 70% or more, and most preferably 100% or more higher than the expression level of a healthy individual.

### 3. Medicament for Preventing or Treating Solid Cancer

### 3.1. Inhibition of Functions or Expression of Solid Cancer Antigenic Polypeptide

Solid cancer antigenic polypeptides are specifically expressed in solid cancer. Thus, such expression very probably causes malignant cell transformation. Therefore, it is expected that therapeutic effects against malignant cell transformation or progress in such transformation can be obtained by inhibiting the functions or the expression of a solid cancer antigenic polypeptide.

Accordingly, a means of inhibiting the functions and the expression of at least one human solid cancer antigenic polypeptides described above is effective as a medicament for preventing and/or treating solid cancer.

Examples of such means of inhibiting the functions or the expression of a human solid cancer antigenic polypeptide include:

(1) an antibody against the solid cancer antigenic polypeptide;

(2) a means capable of inhibiting transcription of a gene encoding the solid cancer antigenic polypeptide; and

(3) a means capable of inhibiting translation of a gene encoding the solid cancer antigenic polypeptide.

### (1) Antibodies Against Solid Cancer Antigenic Polypeptides

Antibodies against solid cancer antigenic polypeptides can inhibit activities of the antigenic polypeptides by specifically binding to solid cancer antigenic polypeptides in a subject. Thus, medicaments comprising antibodies against solid cancer antigenic polypeptides are effective for prevention or therapy of solid cancer.

### (2) Means Capable of Inhibiting Transcription of a Gene Encoding a Solid Cancer Antigenic Polypeptide

Examples of a means of inhibiting transcription of a gene encoding a solid cancer antigenic polypeptide include expression vectors that can be used for substitution of transcriptional promoter regions of the genes in a subject with transcriptional repression-type promoters. In addition, as a means of inhibiting transcription of a gene encoding a solid cancer antigenic polypeptide, expression vectors may be used, with which a nucleotide sequence having transcriptional repression activity is inserted into a region involving translation of the gene. Design and preparation of such expression vectors have been known to persons skilled in the art.

### (3) Means Capable of Inhibiting Translation of a Gene Encoding a Solid Cancer Antigenic Polypeptide

In addition, examples of a means of inhibiting translation of a gene encoding a solid cancer antigenic polypeptide include a method using a so-called antisense RNA. Specifically, a nucleic acid transcribing antisense RNA corresponding to mRNA of a gene is introduced as a plasmid or incorporated into a genome of a subject such that the antisense RNA is allowed to be overexpressed, resulting in inhibition of translation of mRNA of a gene encoding a solid cancer antigenic polypeptide. Techniques related to antisense RNA have been known to be used in cases where mammals and the like are used as hosts (Han et al. (1991) Proc. Natl. Acad. Sci. USA, 88, 4313-4317; Hackett et al. (2000) Plant Physiol., 124, 1079-86).

Further, it is also possible to utilize RNA interference so as to inhibit translation of a gene encoding a solid cancer antigenic polypeptide. Specifically, double-stranded RNA complementary to a nucleotide sequence of a gene encoding a target solid cancer antigenic polypeptide is introduced into a cell such that mRNA of an endogenous gene encoding a solid cancer antigenic polypeptide is degraded, resulting in specific inhibition of gene expression in the cell. Such technique has been found available in the cases of mammalian cells and the like (Hannon, G J., Nature (2002) 418, 244-251 (review); JP Patent Publication No. 2002-516062 A; JP Patent Publication No. 8-506734 A (1996)).

### 3.2. Targeting to Solid Cancer

Since solid cancer antigenic polypeptides are specifically expressed in solid cancer, it becomes possible to allow therapeutic agents for solid cancer to act on cancer lesions with the use of a means of targeting to solid cancer based on such specific expression.

Thus, the means of targeting to solid cancer described above is also effective as a medicament for preventing and/or treating solid cancer. The medicament for preventing and/or treating solid cancer of the present invention includes a gene encoding a prophylactic or therapeutic agent for solid cancer and a means of targeting to human solid cancer.

Examples of such means of targeting to human solid cancer include:

(1) an antibody against a solid cancer antigenic polypeptide; and

(2) a nucleotide sequence of an expression control region of a polynucleotide encoding a solid cancer antigenic polypeptide.

### (1) Antibodies Against Solid Cancer Antigenic Polypeptides

Antibodies against solid cancer antigenic polypeptides bind to the antigenic polypeptides that are specifically expressed in cancer cells. Thus, when known agents for solid cancer therapy (e.g., anticancer drugs or immunopotentiating agents) are linked to such antibodies so as to be administered to patients, it is possible to allow the agents for solid cancer therapy to specifically act on cancer cells.

### (2) Nucleotide Sequences of Expression Control Regions of Polynucleotides Encoding Solid Cancer Antigenic Polypeptides

Expression control regions of polynucleotides encoding solid cancer antigenic polypeptides (hereafter to be referred to as “promoter sequence(s)”) are expression control regions of genes that are specifically expressed in solid cancer cells. Thus, when therapeutic genes are produced by ligating polynucleotides encoding agents for solid cancer therapy to a promoter sequence and are administered in vivo, it becomes possible to allow such therapeutic genes to be expressed in a cancer cell-specific manner. Examples of polynucleotides encoding substances having an anticancer effect or precursors thereof that can be used include DNA and cDNA of genes encoding p53, herpes simplex virus thymidine kinase, interleukin-2, -12, -17, -18, cytosine deaminase, uracil phosphoribosyltransferase, and the like. Moreover, such promoter sequences can be used in a therapeutic method wherein adenovirus and herpes virus are proliferated in a cancer-cell-specific manner such that cancer cells are degraded. That is, by inserting promoter sequence before the adenovirus E1A region, such adenovirus is allowed to specifically proliferate exclusively in cancer cells such that cancer cells are degraded.

### 3.3. Application and Administration of Medicament

Examples of solid cancer to which the medicament of the present invention is applied include, but are not limited to, colorectal cancer, esophageal cancer, gastric cancer, lung cancer, kidney cancer, thyroid cancer, parotid gland cancer, head and neck cancer, soft tissue and bone sarcoma, urinary tract cancer, bladder cancer, uterine cancer, liver cancer, breast cancer, ovarian cancer, and fallopian tube cancer. More specifically, such solid cancer is colorectal cancer, esophageal cancer, gastric cancer, or breast cancer.

It is possible to administer the medicament of the present invention for the purpose of preventing development of the solid cancer described above or preventing deterioration in terms of symptoms or alleviating symptoms of a patient having solid cancer or a patient who has been diagnosed as being at high risk of developing solid cancer.

When the aforementioned means is used as a medicament for treating or preventing solid cancer, it can be mixed with pharmaceutically acceptable carriers so as to be used as a pharmaceutical composition. In such case, an active ingredient of such medicament may be adequately adjusted so as to account for 1% to 90% of the carrier content.

Examples of administration routes of the medicament of the present invention include systemic administration such as intravenous or intra-arterial administration, in general. Further, it is preferable to carry out local administration such as local injection to primary cancer lesions or metastatic lesions that can be predicted based on the cancer type.

The dose of the medicament of the present invention varies depending on age, sex, symptoms, administration routes, administration frequencies, and formulations. These may be adequately adjusted by persons skilled in the art or physicians.

The present invention will be hereafter described in detail with reference to the following examples, although the scope of the present invention is not limited thereto.

### EXAMPLE 1

### Identification of Colorectal Cancer Antigenic Polypeptides Via Two-Dimensional Electrophoresis

### [1] Material and Methods

With the consent of patients (6 cases), frozen specimens of cancer tissue and non-cancer tissue of each patient were separately collected immediately after colorectal cancer extraction. These frozen specimens were kept at −80° C. An adequate amount of each frozen specimen was homogenized with a solution containing 9.5 M Urea, 2% CHAPS, 1% DTT, and complete protease inhibitor cocktail tablets (Roche). The resultant was centrifuged using an ultracentrifuge (Hitachi) at 100,000 g, followed by extraction of the supernatant (protein solution) thereof. Then, protein concentration was determined based on absorbance.

Proteins obtained from cancer tissue and non-cancer tissue (400 μg each) were subjected to two-dimensional electrophoresis for separation. The first-dimensional electrophoresis and the second-dimensional electrophoresis were carried out via agarose isoelectric focusing electrophoresis and 12% or 6% to 10% Tris/Glycine SDS polyacrylamide gel electrophoresis, respectively. The thus separated proteins were stained with Coomassie Brilliant Blue R250, followed by detection of spots indicating an increased protein expression level in cancer tissue compared with that in non-cancer tissue. Then, gel containing the spots was excised, followed by digestion of proteins contained in the gel sections using trypsin (Roche). The thus obtained peptides were collected and subjected to amino acid sequence determination using an ion-trap mass spectrometer (LCQ DECA XP, ThermoQuest).

### [2] Results

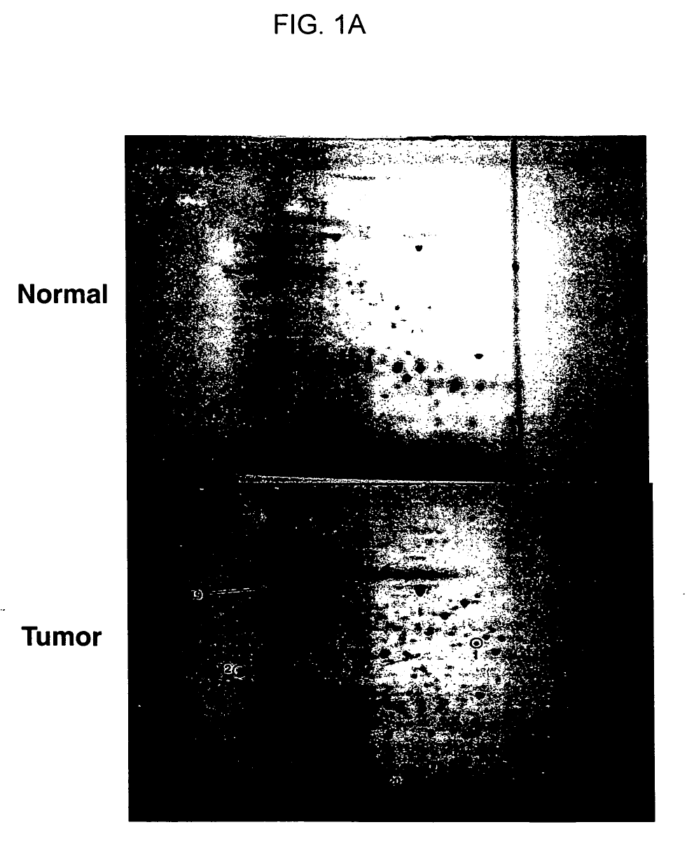

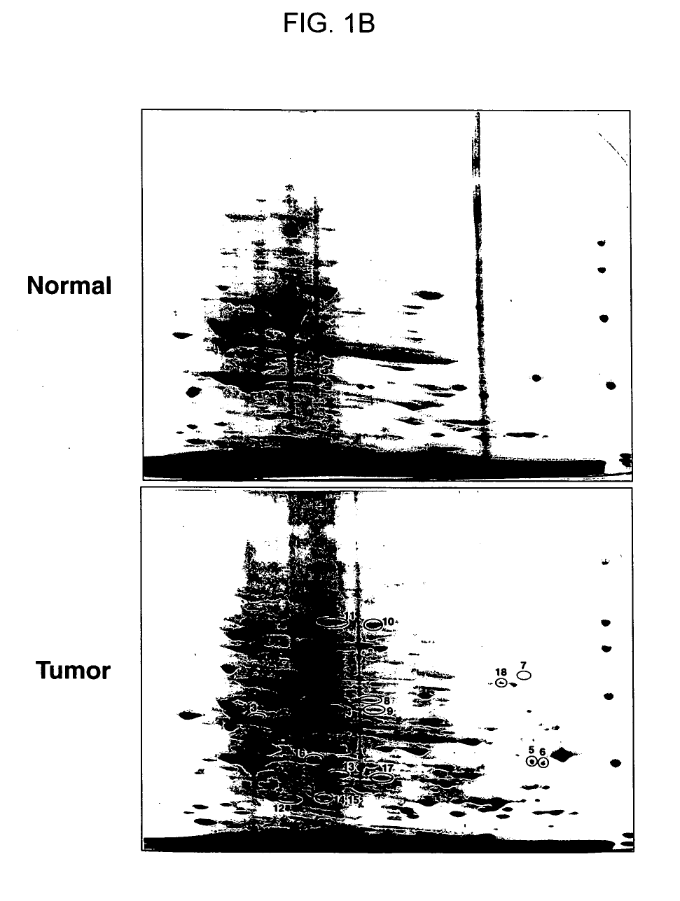

The results are shown in FIGS. 1A and 1B. The comparison between cancer tissue (Tumor) and normal tissue (Normal) of colorectal cancer patients (6 cases) confirmed specific expression of proteins in the cancer tissue in 4 to 6 cases. The expressed proteins had amino acid sequences set forth in SEQ ID NOS: 2, 4, 6, 8, 10, 12, 14, 16, 18, 20, 22, 24, 26, 28, 30, 32, 34, 36, 38, and 40. In FIGS. 1A and 1B, numbers assigned to encircled proteins correspond to antigenic polypeptides denoted by the numbers of 1 to 20 in table 1.

Nucleotide sequences encoding these proteins are set forth in SEQ ID NOS: 1, 3, 5, 7, 9, 11, 13, 15, 17, 19, 21, 23, 25, 27, 29, 31, 33, 35, 37, and 39, respectively. The respective genes are shown in table 1.

### EXAMPLE 2

### Identification of Solid Cancer Antigenic Polypeptides by a SEREX Method

[1] Construction of cDNA Library

A human esophageal-cancer-derived cell line T.Tn was cultured in DMEM medium containing a 10% fetal bovine-serum supplemented with kanamycin (100 μg/ml). Then, total RNA (250 μg) was isolated from these cultured cells via a guanidinium thiocyanate-phenol-chloroform extraction method, followed by 2 repetitions of poly(A) selection using oligo-dT (Oligotex-dT30 super, TAKARA). Thus, mRNA was purified. With the use of the thus obtained mRNA (5.7 μg), a cDNA library of each cell was constructed. Single-stranded cDNA was synthesized using a XhoI linker primer and 5-methyl dCTP. Double-stranded cDNA having blunt ends was synthesized using T4 DNA polymerase based on the single-stranded cDNA. To both ends of the double-stranded cDNA, a linker containing restriction enzyme sites (EcoRI/λZAPII) was added. The obtained cDNA fragment was inserted into a bacteriophage (Stratagene). Then, a cDNA library of each cancer cell comprising about 1.8×10⁶ clones was constructed.

[2] Screening of cDNA Library

Phage vectors of the above-prepared cDNA libraries of cancer cells were allowed to infect E coli. XL1-Blue. Then, plaque was formed on an NZY agarose plate. Each infected E coli. cell was treated with 10 mM IPTG, resulting in expression induction. Thus, peptides encoded by the various cDNAs were expressed. These peptides were transferred to a nitrocellulose filter (NitroBind, Osmonics). The filter was washed with TBS (0.5% Tween20-containing TBS (10 mM Tris-HCl, 150 mM NaCl; pH 7.5)) such that bacteriophages that had adsorbed thereto were removed. Thereafter, nonspecific reaction was suppressed using TBS-Tween containing 1% albumin. The filter was subjected to reaction with serum of patients with esophageal cancer, gastric cancer, colorectal cancer, and breast cancer at room temperature for 2 hours.

The serum isolated from each patient was kept at −80° C. and diluted 500-fold immediately before use with a TBS-Tween solution (TBS-Tween containing 0.5% polyoxyethylene sorbitan monolaurate) that contained 1% by weight albumin. The thus diluted serum was mixed with an E coli. lysate at a ratio of 1:5. The resultant was allowed to stand at 4° C. for 8 hours, followed by centrifugation at 15,000 g for 20 minutes. Then, the supernatant thereof was recovered and used. In addition, untreated serum was diluted 2000-fold and used according to need.

Each serum was subjected to reaction with the aforementioned nitrocellulose filter on which expressed peptides had been blotted at room temperature for 10 to 20 hours. Thus, polypeptides with which serum antibodies had reacted were identified. Specifically, alkaline-phosphatase-labeled anti human IgG-F (ab′)₂ goat antibodies (Jachson) were diluted 5000-fold and used as secondary antibodies during the reaction. Label signals were detected via enzymatic chromogenic reaction using nitroblue tetrazolium (Wako) and 5-bromo-4-chloro-3-indolyl phosphate (Wako). Colonies corresponding to chromogenic-reaction-positive were collected from the agarose plate and dissolved in SM buffer (100 mM NaCl, 10 mM MgSO₄, and 50 mM Tris-HCl; pH 7.5). Until chromogenic-reaction-positive colonies became united, secondary screening and tertiary screening were repeated as described above. Thus, phage clones that reacted with serum IgG of 5 patients were subjected to screening such that positive clones were isolated.

### [3] Identification of Novel Antigens

With the use of the obtained positive clones, insert DNA was replicated by PCR. The resulting product was subjected to sequencing using a Big Dye DNA Sequencing Kit (ABI) and ABI Prism (Perkin Elmer). As a result of searching based on existing databases, in addition to antigenic polypeptides that were expression products of known cancer-related genes, 19 types of novel antigenic polypeptides that reacted with serum antibodies of a plurality of patients were identified. Table 2 shows antibody prevalences among cancer patients to these novel antigenic polypeptides.

TABLE 2

| | | | | Antibody prevalence (%) | | | | | |
|---|---|---|---|---|---|---|---|---|---|
| | | | | | Early | | | | |
| | | | | Esophageal | esophageal | Colorectal | Gastric | Breast | |
| | Clone | | Registered | cancer | cancer | cancer | cancer | cancer | Healthy |
| No. | name | Name | number | patient | patient | patient | patient | patient | individual |
| 1 | K35-1-1 | Progesterone receptor | NM_006320 | 10 | 13 | 0 | 20 | 0 | 10 |
| | | membrane component 2 | | | | | | | |
| 2 | K30-1-1 | MAP kinase interacting | NM_199054 | 0 | 13 | 10 | 20 | 20 | 5 |
| | | serine/threonine kinase 2 | | | | | | | |
| 3 | 12N3-1 | EST: 601191782F1 | BE264462 | 5 | 0 | 0 | 0 | 0 | 0 |
| 4 | 12O1-1 | EST: 602301679F1 | BG032310 | 15 | 0 | 10 | 30 | 0 | 0 |
| 5 | 14A1-1-1 | Additional sex | NM_015338 | 15 | 13 | 10 | 10 | 0 | 6 |
| | | combs-like 1 | | | | | | | |
| 6 | 18G3-1 | Forkhead box A1 | NM_004496 | 10 | 0 | 0 | 0 | 20 | 0 |
| 7 | 19C1-1 | Retinoic acid induced 16 | NM_022749 | 5 | 0 | 0 | 0 | 10 | 0 |
| 8 | 19F1-1 | RIKEN cDNA | NM_080655 | 5 | 0 | 40 | 10 | 10 | 5 |
| | | 5730528L13-like gene | | | | | | | |
| 9 | 19F1-2 | Lysine tRNA synthase | BC004132 | 0 | 13 | 20 | 20 | 20 | 0 |
| 10 | 6BD3-1 | EST: | CF597227 | 14 | | | | | 0 |
| | | AGENCOURT_15657942 | | | | | | | |
| 11 | 14H1-2-1 | KDEL endoplasmic | NM_006801 | 10 | | | | | 0 |
| | | reticulum protein | | | | | | | |
| | | retention receptor 1 | | | | | | | |
| 12 | 18B2-1 | Lysosomal associated | NM_018407 | 10 | | | | | 0 |
| | | protein | | | | | | | |
| | | transmembrane 4 beta | | | | | | | |
| 13 | 18G1-1 | Protein phosphatase | NM_002708 | 10 | | | | | 0 |
| | | 1, catalytic subunit, | | | | | | | |
| | | α isoform | | | | | | | |
| 14 | 20J4-1 | Peroxyredoxin 3 | NM_006793 | 14 | | | | | 0 |
| 15 | 19M2 | Aldo-keto reductase | NM_003739 | 38 | | | | | 0 |
| | | family 1, member C3 | | | | | | | |
| 16 | 10Q3-1 | Ubiquitin-conjugating | BC000744 | 33 | | | | | 0 |
| | | enzyme E2I | | | | | | | |
| 17 | 14A1-1-2 | Phosphatidic acid | NM_003712 | 40 | | | | | 21 |
| | | phosphatase, Type 2C | | | | | | | |
| 18 | 14B1-2-1 | Beta-catenin interacting | NM_020248 | 33 | | | | | 17 |
| | | protein 1 | | | | | | | |
| 19 | 14H2-1-1 | Sorting nexin 15 | NM_147777 | 11 | | | | | 4 |

Polynucleotide (cDNA) sequences that encode amino acids of these 19 types of novel antigenic polypeptides have nucleotide sequences set forth in SEQ ID NOS: 41, 43, 45, 46, 47, 49, 51, 53, 55, 57, 58, 60, 62, 64, 66, 68, 70, 72, and 74, respectively. In addition, nucleotide sequences set forth in SEQ ID NOS: 41, 43, 47, 49, 51, 53, 55, 58, 60, 62, 64, 66, 68, 70, 72, and 74 have amino acid sequences set forth in SEQ ID NOS: 42, 44, 48, 50, 52, 54, 56, 59, 61, 63, 65, 67, 69, 71, 73, and 75, respectively.

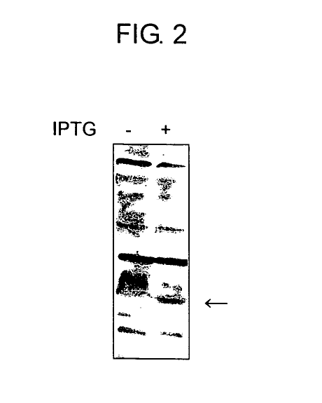

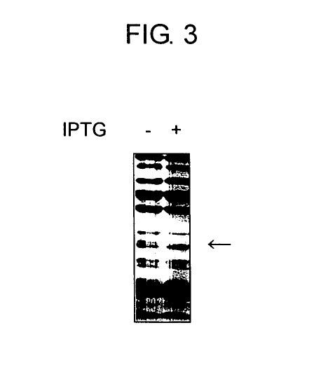

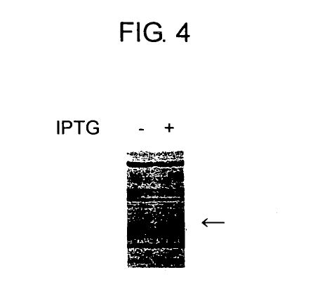

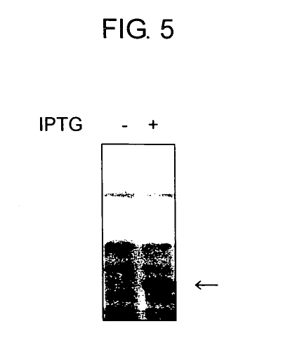

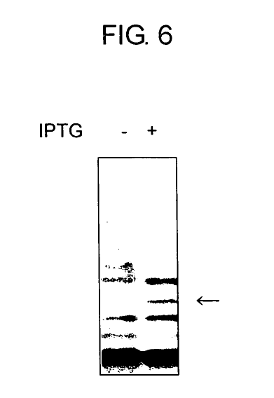

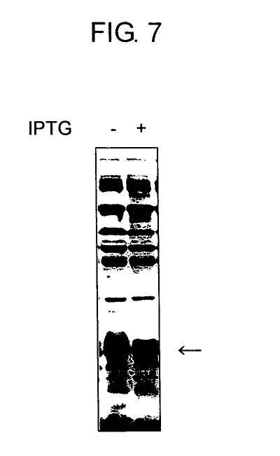

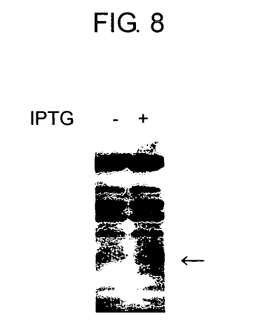

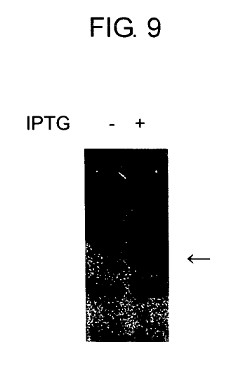

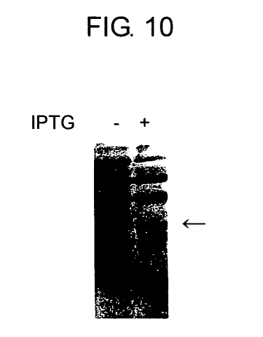

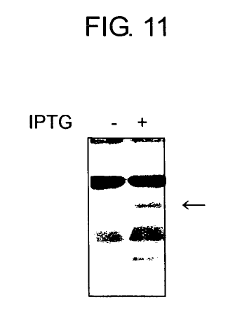

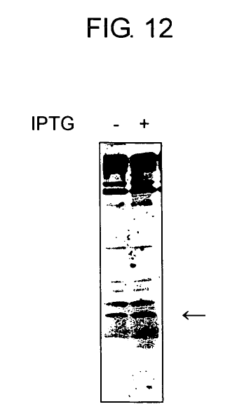

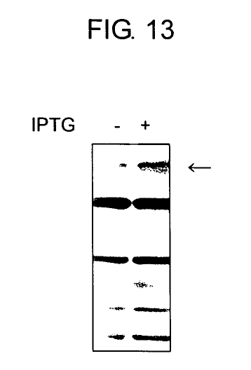

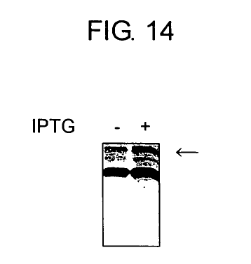

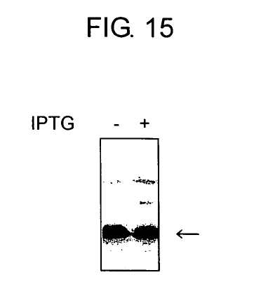

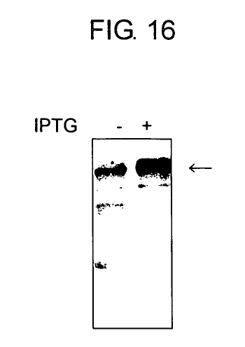

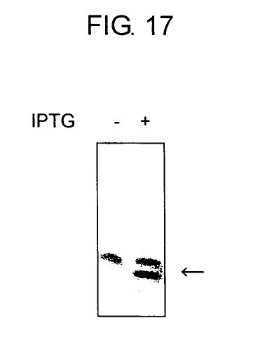

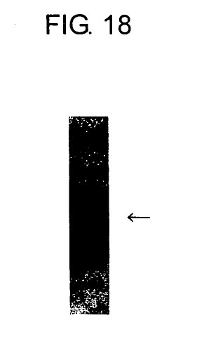

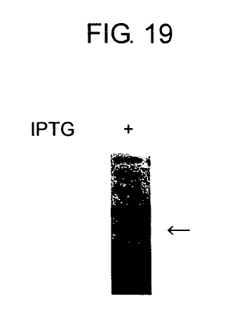

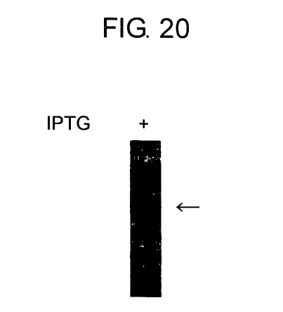

FIGS. 2 to 20 show the results of Western blotting analysis that was carried out to examine binding reactions involving these 19 types of novel antigenic polypeptides and serum antibodies of the patients. In FIGS. 2 to 20, arrows indicate polypeptides that underwent specific reaction with the serum antibodies of the patients. The polypeptides were detected in the IPTG-treated E coli. extract; however, they were not detected in the untreated E coli. extract. Thus, the polypeptides were confirmed to be derived from cDNA that had been introduced thereinto.

All publications, patents, and patent applications cited herein are incorporated herein by reference in their entirety.

### INDUSTRIAL APPLICABILITY

According to the diagnostic kit for solid cancer of the present invention, solid cancer can be diagnosed with high accuracy and it is useful for early diagnosis of solid cancer. In addition, with the use of the medicament for preventing or treating solid cancer of the present invention, it becomes possible to carry out a therapy whereby selective targeting of solid cancer is carried out.

1. A human solid cancer antigenic polypeptide having an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 48, 50, 52, 54, 56, 59, 61, 63, 65, 67, 69, 71, 73, and 75.

2. A polynucleotide encoding the human solid cancer antigenic polypeptide according to claim 1.

3. A polynucleotide having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 46, 47, 49, 51, 53, 55, 57, 58, 60, 62, 64, 66, 68, 70, 72, and 74.

4. A diagnostic kit for solid cancer comprising a means of detecting the expression of at least one human solid cancer antigenic polypeptide in a sample derived from a subject, characterized in that the human solid cancer antigenic polypeptide has an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 48, 50, 52, 54, 56, 59, 61, 63, 65, 67, 69, 71, 73, and 75.

5. A diagnostic kit for solid cancer comprising a means of detecting the expression of at least one human solid cancer antigenic polypeptide in a sample derived from a subject, characterized in that the human solid cancer antigenic polypeptide is encoded by a polynucleotide having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 46, 47, 49, 51, 53, 55, 57, 58, 60, 62, 64, 66, 68, 70, 72, and 74.

6. The diagnostic kit for solid cancer according to claim 4, wherein the means of detecting the expression of the human solid cancer antigenic polypeptide is the solid cancer antigenic polypeptide or a partial peptide thereof.

7. The diagnostic kit for solid cancer according to claim 4, wherein the means of detecting the expression of the human solid cancer antigenic polypeptide is an antibody against the solid cancer antigenic polypeptide.

8. The diagnostic kit for solid cancer according to claim 4, wherein the means of detecting the expression of the human solid cancer antigenic polypeptide is a primer or probe comprising a polynucleotide consisting of the entire or a partial sequence of a polynucleotide encoding the solid cancer antigenic polypeptide or a complementary sequence thereof.

9. The diagnostic kit for solid cancer according to claim 4, wherein the means of detecting the expression of the human solid cancer antigenic polypeptide is immobilized on a solid phase.

10. The diagnostic kit for solid cancer according to claim 4, wherein the means of detecting the expression of the human solid cancer antigenic polypeptide is labeled.

11. The diagnostic kit for solid cancer according to claim 4, wherein the solid cancer is selected from the group consisting of colorectal cancer, esophageal cancer, gastric cancer, and breast cancer.

12. The diagnostic kit for solid cancer according to claim 4, wherein the sample is selected from the group consisting of serum, blood, hemocytes, and tissue.

13. A medicament for preventing or treating solid cancer comprising a means of inhibiting the functions or expression of at least one human solid cancer antigenic polypeptide, characterized in that the human solid cancer antigenic polypeptide has an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 48, 50, 52, 54, 56, 59, 61, 63, 65, 67, 69, 71, 73, and 75.

14. A medicament for preventing or treating solid cancer comprising a means of inhibiting the functions or expression of at least one human solid cancer antigenic polypeptide, characterized in that the human solid cancer antigenic polypeptide is encoded by a polynucleotide having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 46, 47, 49, 51, 53, 55, 57, 58, 60, 62, 64, 66, 68, 70, 72, and 74.

15. The medicament according to claim 13, wherein the means of inhibiting the functions or expression of the human solid cancer antigenic polypeptide is an antibody against the solid cancer antigenic polypeptide.

16. The medicament according to claim 13, wherein the means of inhibiting the functions or expression of the human solid cancer antigenic polypeptide is a means capable of inhibiting transcription of a gene encoding the solid cancer antigenic polypeptide.

17. The medicament according to claim 13, wherein the means of inhibiting the functions or expression of the human solid cancer antigenic polypeptide is a means capable of inhibiting translation of a gene encoding the solid cancer antigenic polypeptide.

18. A medicament for preventing or treating solid cancer comprising a gene encoding a prophylactic or therapeutic agent for solid cancer and a means of targeting to human solid cancer.

19. The medicament according to claim 18, wherein the means of targeting to human solid cancer is an antibody against a solid cancer antigenic polypeptide.

20. The medicament according to claim 18, wherein the means of targeting to human solid cancer is a nucleotide sequence of an expression control region of a polynucleotide encoding a solid cancer antigenic polypeptide.
